SEQUENCE LISTING (103 sequences)

SEQ ID NO: 1 - RNA, 20 nt, synthetic construct
  misc_feature 1..20: note = Description of sequence: Upstream gRNA #1
  source 1..20: mol_type = other RNA
gaaacttgcagctaactggg

SEQ ID NO: 2 - RNA, 20 nt, synthetic construct
  misc_feature 1..20: note = Description of sequence: Upstream gRNA #2
  source 1..20: mol_type = other RNA
gttagtagcaccgacactaa

SEQ ID NO: 3 - RNA, 20 nt, synthetic construct
  misc_feature 1..20: note = Description of sequence: Upstream gRNA #3
  source 1..20: mol_type = other RNA
tcagggaccagtattcgagg

SEQ ID NO: 4 - RNA, 20 nt, synthetic construct
  misc_feature 1..20: note = Description of sequence: Upstream gRNA #4
  source 1..20: mol_type = other RNA
aatatggtctaccggaagag

SEQ ID NO: 5 - RNA, 20 nt, synthetic construct
  misc_feature 1..20: note = Description of sequence: Upstream gRNA #5
  source 1..20: mol_type = other RNA
acctgtaagtgacattacac

SEQ ID NO: 6 - RNA, 20 nt, synthetic construct
  misc_feature 1..20: note = Description of sequence: Downstream gRNA #1
  source 1..20: mol_type = other RNA
tcttcgaaaagccacactgg

SEQ ID NO: 7 - RNA, 20 nt, synthetic construct
  misc_feature 1..20: note = Description of sequence: Downstream gRNA #2
  source 1..20: mol_type = other RNA
catctcaacaagaaagtcgg

SEQ ID NO: 8 - RNA, 20 nt, synthetic construct
  misc_feature 1..20: note = Description of sequence: Downstream gRNA #3
  source 1..20: mol_type = other RNA
ctcctaggctccaatatggg

SEQ ID NO: 9 - RNA, 20 nt, synthetic construct
  misc_feature 1..20: note = Description of sequence: Downstream gRNA #4
  source 1..20: mol_type = other RNA
gaagactgaatgctcacggt

SEQ ID NO: 10 - RNA, 20 nt, synthetic construct
  misc_feature 1..20: note = Description of sequence: Downstream gRNA #5
  source 1..20: mol_type = other RNA
aatgcacgcatgacttgaag

SEQ ID NO: 11 - DNA, 23 bp, synthetic construct
  misc_feature 1..23: note = Description of sequence: Upstream target DNA #1
  source 1..23: mol_type = other DNA
  misc_feature 20..23: note = PAM Sequence
gaaacttgcagctaactgggagg

SEQ ID NO: 12 - DNA, 23 bp, synthetic construct
  misc_feature 1..23: note = Description of sequence: Upstream target DNA #2
  source 1..23: mol_type = other DNA
  misc_feature 20..23: note = PAM Sequence
gttagtagcaccgacactaaggg

SEQ ID NO: 13 - DNA, 23 bp, synthetic construct
  misc_feature 1..23: note = Description of sequence: Upstream target DNA #3
  source 1..23: mol_type = other DNA
  misc_feature 20..23: note = PAM Sequence
tcagggaccagtattcgaggagg

SEQ ID NO: 14 - DNA, 23 bp, synthetic construct
  misc_feature 1..23: note = Description of sequence: Upstream target DNA #4
  source 1..23: mol_type = other DNA
  misc_feature 20..23: note = PAM Sequence
aatatggtctaccggaagagggg

SEQ ID NO: 15 - DNA, 23 bp, synthetic construct
  misc_feature 1..23: note = Description of sequence: Upstream target DNA #5
  source 1..23: mol_type = other DNA
  misc_feature 20..23: note = PAM Sequence
acctgtaagtgacattacactgg

SEQ ID NO: 16 - DNA, 23 bp, synthetic construct
  misc_feature 1..23: note = Description of sequence: Downstream target DNA #1
  source 1..23: mol_type = other DNA
  misc_feature 20..23: note = PAM Sequence
tcttcgaaaagccacactggagg

SEQ ID NO: 17 - DNA, 23 bp, synthetic construct
  misc_feature 1..23: note = Description of sequence: Downstream target DNA #2
  source 1..23: mol_type = other DNA
  misc_feature 20..23: note = PAM Sequence
catctcaacaagaaagtcggagg

SEQ ID NO: 18 - DNA, 23 bp, synthetic construct
  misc_feature 1..23: note = Description of sequence: Downstream target DNA #3
  source 1..23: mol_type = other DNA
  misc_feature 20..23: note = PAM Sequence
ctcctaggctccaatatgggggg

SEQ ID NO: 19 - DNA, 23 bp, synthetic construct
  misc_feature 1..23: note = Description of sequence: Downstream target DNA #4
  source 1..23: mol_type = other DNA
  misc_feature 20..23: note = PAM Sequence
gaagactgaatgctcacggttgg

SEQ ID NO: 20 - DNA, 23 bp, synthetic construct
  misc_feature 1..23: note = Description of sequence: Downstream target DNA #5
  source 1..23: mol_type = other DNA
  misc_feature 20..23: note = PAM Sequence
aatgcacgcatgacttgaagtgg

SEQ ID NO: 21 - DNA, 21 bp, synthetic construct
  misc_feature 1..21: note = Description of sequence: Forward Primer chr13:102161798-102162411
  source 1..21: mol_type = other DNA
tggaaatttgctctgtgggct

SEQ ID NO: 22 - DNA, 21 bp, synthetic construct
  misc_feature 1..21: note = Description of sequence: Reverse Primer chr13:102161798-102162411
  source 1..21: mol_type = other DNA
cgttcccaatatagtgcgcct

SEQ ID NO: 23 - DNA, 20 bp, synthetic construct
  misc_feature 1..20: note = Description of sequence: Forward Primer chr13:102162735-102163346
  source 1..20: mol_type = other DNA
aacccctcttccggtagacc

SEQ ID NO: 24 - DNA, 22 bp, synthetic construct
  misc_feature 1..22: note = Description of sequence: Reverse Primer chr13:102162735-102163346
  source 1..22: mol_type = other DNA
agctgtttctctgggcctctcc

SEQ ID NO: 25 - DNA, 22 bp, synthetic construct
  misc_feature 1..22: note = Description of sequence: Forward Primer chr13:102163461-102163873
  source 1..22: mol_type = other DNA
tccagactttaagtggcctcca

SEQ ID NO: 26 - DNA, 23 bp, synthetic construct
  misc_feature 1..23: note = Description of sequence: Reverse Primer chr13:102163461-102163873
  source 1..23: mol_type = other DNA
ggaagtttagagcaagtggaagg

SEQ ID NO: 27 - DNA, 20 bp, synthetic construct
  misc_feature 1..20: note = Description of sequence: Forward Primer chr13:102163112-102163711
  source 1..20: mol_type = other DNA
acttccccaccctacaccct

SEQ ID NO: 28 - DNA, 23 bp, synthetic construct
  misc_feature 1..23: note = Description of sequence: Reverse Primer chr13:102163112-102163711
  source 1..23: mol_type = other DNA
gcttccaaacatagccagactct

SEQ ID NO: 29 - DNA, 23 bp, synthetic construct
  misc_feature 1..23: note = Description of sequence: Forward Primer chr13:102163705-102164383
  source 1..23: mol_type = other DNA
tggtcatcagtaaacacagtgtc

SEQ ID NO: 30 - DNA, 20 bp, synthetic construct
  misc_feature 1..20: note = Description of sequence: Reverse Primer chr13:102163705-102164383
  source 1..20: mol_type = other DNA
gggaagtgaccaagggagcc

SEQ ID NO: 31 - DNA, 23 bp, synthetic construct
  misc_feature 1..23: note = Description of sequence: Forward Primer chr13:102159382-102159777
  source 1..23: mol_type = other DNA
agcatcactagaaattcagccaa

SEQ ID NO: 32 - DNA, 22 bp, synthetic construct
  misc_feature 1..22: note = Description of sequence: Reverse Primer chr13:102159382-102159777
  source 1..22: mol_type = other DNA
tgggatagcaaacacggcatca

SEQ ID NO: 33 - DNA, 21 bp, synthetic construct
  misc_feature 1..21: note = Description of sequence: Forward Primer chr13:102160004-102160248
  source 1..21: mol_type = other DNA
tctgaggaagcgtggaggatg

SEQ ID NO: 34 - DNA, 23 bp, synthetic construct
  misc_feature 1..23: note = Description of sequence: Reverse Primer chr13:102160004-102160248
  source 1..23: mol_type = other DNA
aggtccatttacagcttcagtct

SEQ ID NO: 35 - DNA, 22 bp, synthetic construct
  misc_feature 1..22: note = Description of sequence: Forward Primer chr13:102160458-102161107
  source 1..22: mol_type = other DNA
acattctgccttcctccattgt

SEQ ID NO: 36 - DNA, 21 bp, synthetic construct
  misc_feature 1..21: note = Description of sequence: Reverse Primer chr13:102160458-102161107
  source 1..21: mol_type = other DNA
tctcacacacccggctctctg

SEQ ID NO: 37 - DNA, 20 bp, synthetic construct
  misc_feature 1..20: note = Description of sequence: Forward Primer chr13:102161367-102161495
  source 1..20: mol_type = other DNA
ggcaagcttacactgactga

SEQ ID NO: 38 - DNA, 20 bp, synthetic construct
  misc_feature 1..20: note = Description of sequence: Reverse Primer chr13:102161367-102161495
  source 1..20: mol_type = other DNA
actggagtaactgtggctgt

SEQ ID NO: 39 - DNA, 23 bp, synthetic construct
  misc_feature 1..23: note = Description of sequence: Forward Primer chr13:102164666-102164812
  source 1..23: mol_type = other DNA
ccagcactgtccaatagaacttt

SEQ ID NO: 40 - DNA, 23 bp, synthetic construct
  misc_feature 1..23: note = Description of sequence: Reverse Primer chr13:102164666-102164812
  source 1..23: mol_type = other DNA
ggtagttaatgtctgtgctgtcc

SEQ ID NO: 41 - DNA, 21 bp, synthetic construct
  misc_feature 1..21: note = Description of sequence: Long-range Forward Primer
  source 1..21: mol_type = other DNA
tgcaaatgaaggaaaactctt

SEQ ID NO: 42 - DNA, 22 bp, synthetic construct
  misc_feature 1..22: note = Description of sequence: Long-range Reverse Primer
  source 1..22: mol_type = other DNA
caatgatgaattaagcagttcc

SEQ ID NO: 43 - DNA, 21 bp, synthetic construct
  misc_feature 1..21: note = Description of sequence: Repeat Primed Forward Primer
  source 1..21: mol_type = other DNA
tgcccacatagagcttagtct

SEQ ID NO: 44 - DNA, 40 bp, synthetic construct
  misc_feature 1..40: note = Description of sequence: Repeat Primed Reverse Primer
  source 1..40: mol_type = other DNA
cacgacgttgtaaaacgacgaagaagaagaagaagaagaa

SEQ ID NO: 45 - DNA, 19 bp, synthetic construct
  misc_feature 1..19: note = Description of sequence: M13-FAM
  source 1..19: mol_type = other DNA
  modified_base 1: mod_base = OTHER; note = 5' Carboxyfluorescein
cacgacgttgtaaaacgac

SEQ ID NO: 46 - DNA, 18 bp, synthetic construct
  misc_feature 1..18: note = Description of sequence: ACTB Forward Primer
  source 1..18: mol_type = other DNA
attggcaatgagcggttc

SEQ ID NO: 47 - DNA, 19 bp, synthetic construct
  misc_feature 1..19: note = Description of sequence: GAPDH Forward Primer
  source 1..19: mol_type = other DNA
agccacatcgctcagacac

SEQ ID NO: 48 - DNA, 27 bp, synthetic construct
  misc_feature 1..27: note = Description of sequence: HPRT1 Forward Primer
  source 1..27: mol_type = other DNA
tgatagatccattcctatgactgtaga

SEQ ID NO: 49 - DNA, 25 bp, synthetic construct
  misc_feature 1..25: note = Description of sequence: YWHAZ Forward Primer
  source 1..25: mol_type = other DNA
gcaattactgagagacaacttgaca

SEQ ID NO: 50 - DNA, 20 bp, synthetic construct
  misc_feature 1..20: note = Description of sequence: RPL13 Forward Primer
  source 1..20: mol_type = other DNA
cacccagggagctgttactg

SEQ ID NO: 51 - DNA, 20 bp, synthetic construct
  misc_feature 1..20: note = Description of sequence: UBE2D2 Forward Primer
  source 1..20: mol_type = other DNA
ttgaatgatctggcacggga

SEQ ID NO: 52 - DNA, 21 bp, synthetic construct
  misc_feature 1..21: note = Description of sequence: FGF14 Forward Primer
  source 1..21: mol_type = other DNA
gcaagctatgaaagggaacag

SEQ ID NO: 53 - DNA, 21 bp, synthetic construct
  misc_feature 1..21: note = Description of sequence: ACTB Reverse Primer
  source 1..21: mol_type = other DNA
tgaaggtagtttcgtggatgc

SEQ ID NO: 54 - DNA, 19 bp, synthetic construct
  misc_feature 1..19: note = Description of sequence: GAPDH Reverse Primer
  source 1..19: mol_type = other DNA
gcccaatacgaccaaatcc

SEQ ID NO: 55 - DNA, 26 bp, synthetic construct
  misc_feature 1..26: note = Description of sequence: HPRT1 Reverse Primer
  source 1..26: mol_type = other DNA
caagacattctttccagttaaagttg

SEQ ID NO: 56 - DNA, 19 bp, synthetic construct
  misc_feature 1..19: note = Description of sequence: YWHAZ Reverse Primer
  source 1..19: mol_type = other DNA
tggaaggccggttaatttt

SEQ ID NO: 57 - DNA, 20 bp, synthetic construct
  misc_feature 1..20: note = Description of sequence: RPL13 Reverse Primer
  source 1..20: mol_type = other DNA
caagcaaggtgtcatcgtgt

SEQ ID NO: 58 - DNA, 21 bp, synthetic construct
  misc_feature 1..21: note = Description of sequence: UBE2D2 Reverse Primer
  source 1..21: mol_type = other DNA
gccccattattgtagcttgcc

SEQ ID NO: 59 - DNA, 20 bp, synthetic construct
  misc_feature 1..20: note = Description of sequence: FGF14 Reverse Primer
  source 1..20: mol_type = other DNA
gtgcttttacttggcgtcac

SEQ ID NO: 60 - DNA, 24 bp, synthetic construct
  misc_feature 1..24: note = Description of sequence: FGF14 Forward Primer
  source 1..24: mol_type = other DNA
tattgcaggcaaggctactacttg

SEQ ID NO: 61 - DNA, 22 bp, synthetic construct
  misc_feature 1..22: note = Description of sequence: FGF14 Forward Primer
  source 1..22: mol_type = other DNA
catatgctgcagtgtctttgtg

SEQ ID NO: 62 - DNA, 22 bp, synthetic construct
  misc_feature 1..22: note = Description of sequence: FGF14 Forward Primer
  source 1..22: mol_type = other DNA
gcaacctggtggatatcttctc

SEQ ID NO: 63 - DNA, 23 bp, synthetic construct
  misc_feature 1..23: note = Description of sequence: FGF14 Reverse Primer
  source 1..23: mol_type = other DNA
gttttcactccctggatggcaac

SEQ ID NO: 64 - DNA, 21 bp, synthetic construct
  misc_feature 1..21: note = Description of sequence: FGF14 Reverse Primer
  source 1..21: mol_type = other DNA
gtagtagccttgcctgcaata

SEQ ID NO: 65 - DNA, 21 bp, synthetic construct
  misc_feature 1..21: note = Description of sequence: FGF14 Reverse Primer
  source 1..21: mol_type = other DNA
acctggtcactatacccttga

SEQ ID NO: 66 - DNA, 468 bp, synthetic construct
  misc_feature 1..468: note = Description of sequence: PCR Products from Cas9-stable HEK cells transfected with dual-guide RNA pairing, Up1 + Down1
  source 1..468: mol_type = other DNA
cacggcatgtttgtattttggcacttaatcaaatgcatgttaaattctactgtgggtcaggtaccatgcaggatgcttggcatacaaaaataaataagataggatctcataaggcaataagcacacaattatgctacagtttgataggtgggacaacagaagagtgcacgatgagctctgcctccgactttcttgttgagatgtcttcgaaaagccacttttcactcattctccaggtttttttttttttttattttttttttaggcccattaaatggggaaggtatctttctcaaaaaactttttgctccggttatattctactttataaaggaatattcacccctcctaggggcccctgaagggggttcttaaaacccatctgctggcctttaggaaaaaaaaaggaaaacctttggggttttccttaataaaggggtgcatttttgggggatctcagacccagac

SEQ ID NO: 67 - DNA, 504 bp, synthetic construct
  misc_feature 1..504: note = Description of sequence: PCR Products from Cas9-stable HEK cells transfected with dual-guide RNA pairing, Up1+ Down2
  source 1..504: mol_type = other DNA
caacacggaatgatgtatttggcacttaatcaaatgcatgttaaattctactgtgggtcaggtaccatgcaggatgcttggcatacaaaaataaataagataggatctcataaggcaataagcacacaattatgctacagtttgataggtgggacaacagaagagtgcacgatgagctctgcctccgagttagctgcaacattcccaaacttgttctaatccctccccttaccatcttttaaaaaacaggagataactcctaaaaaaacttgttaactgacaaaatgtctcccaagtgtcccatttttgttgttccatttatattctaatctataaagtaatattaacacatcagaaaggctcatggaggtgtttcttgaaaccctactgcttaccttaaggataaaaaaaagaaaacagttataattttccttgataaaaagattaaatttagggccatatagtgagcaatgtcattcactcattctccaggttttttttttt

SEQ ID NO: 68 - DNA, 512 bp, synthetic construct
  misc_feature 1..512: note = Description of sequence: PCR Products from Cas9-stable HEK cells transfected with dual-guide RNA pairing,, Up1 + Down4
  source 1..512: mol_type = other DNA
tctcgttataagggagaagccgggggtaagatctcaaaattggcgaggaagggcggccagggcggtcgaatatgtttcagttctcagagggagggaattcgtacacagaaagggcgggaatcttcgctagtgtattcacactacattttgagctgaaactgaatggaaaagttggatgagagcttcattcattgttttaccactttataagcactgtttttttttgccaccaaaaataaatggggccaaccagttaactgcaaggttcaaaaacttgttctattccctcctcttatcatcttttaaaaaacaggagataactcctaaaaaagcttgttaactgacaaaatgtctcccaaatgtcaaatttttgttgtttcatttatattctaaattataaagtaatattaacacatcagaaatttcatgggaggtgtttcttgaaaccctactgcttacctttaagataaaaaaaaagaaaacattataattttccttgaaaaaaaaattaa

SEQ ID NO: 69 - DNA, 503 bp, synthetic construct
  misc_feature 1..503: note = Description of sequence: PCR Products from Cas9-stable HEK cells transfected with dual-guide RNA pairing,, Up1 + Down5
  source 1..503: mol_type = other DNA
ctcgggaagccgttactccagtggcaactggaacggcaggattatttttcccttgagaatgataataattcattgtttagagggaggaaaatcttccacagaacatggacctgaaattctgacaaccgctcttccttttttcctctctttctttctccttaatttattttttgttgtacttatgcattcatctcttcaagatgtttacttagtacctgatacattgtagatattttcacattaatgatccatgtaaattttatgagaaacagttttcctttacattctgccttcctccattgtttctttctctctcttaggaatactttgtttattttcctctctattttttccatattgatttgaactatcaatctgacttttaatatcctctaagcagaaatgcacgcatgacttgagttagctgcaagtttcagaaacttgttctagtccctcctcttagcatcttttaaaaaacaggagataactcctaaaaaagcttg

SEQ ID NO: 70 - DNA, 425 bp, synthetic construct
  misc_feature 1..425: note = Description of sequence: PCR Products from Cas9-stable HEK cells transfected with dual-guide RNA pairing,, Up2 + Down1
  source 1..425: mol_type = other DNA
caatggtgttgtaatttggcacttaatcaaatgcatgttaaattctactgtgggtcaggtaccatgcaggatgcttggccatacaaaaataaataagataggatctcataaggcaataagcacacaattatgctacagtttgataggtgggacaacagaagagtgcacgatgagctctgcctccgactttcttgttgagatgtcttcgaaaagccacacgtgtcggtgctactaacctacaaatgagggggacgctgctaaccactgccagtgaccagaaaaagagctaccagacttctagtatcctcccaaaaaggcttcactgcacatacaatagaaaaaacctggcagctcatttcaacacaaggctggggtacattttccacagcattcaactcactggagaggcccagagaaacagctaa

SEQ ID NO: 71 - DNA, 504 bp, synthetic construct
  misc_feature 1..504: note = Description of sequence: PCR Products from Cas9-stable HEK cells transfected with dual-guide RNA pairing,, Up2 +Down5
  source 1..504: mol_type = other DNA
ccagggaagcacgttactccagtggcaactgggaacggcaggattatttttcccttgagaatgataataattcattgtttagagggaggaaaatcttccacagaacatggacctgaaattctgacaaccgctcttccttttttcctctctttctttctccttaatttattttttgttgtacttatgcattcatctcttcaagatgtttacttagtacctgatacattgtagatattttcacattaatgatccatgtaaattttatgagaaacaattttcctttacattctgccttcctccattgtttctttctctctcttaggaatactttgtttattttcctctctattttttccatattgatttgaactatcaatatgacttttaatatcctctaagcagaaatgcacgcatgacttggtgtcggtgctactaacctacatacaaatgagggggacgctgctaaccactgccagggaccaaaaaaaaagctaccaaacttct

SEQ ID NO: 72 - DNA, 504 bp, synthetic construct; identical to SEQ ID NO: 71
  misc_feature 1..504: note = Description of sequence: PCR Products from Cas9-stable HEK cells transfected with dual-guide RNA pairing, Up3 +Down4
  source 1..504: mol_type = other DNA

SEQ ID NO: 73 - DNA, 516 bp, synthetic construct
  misc_feature 1..516: note = Description of sequence: PCR Products from Cas9-stable HEK cells transfected with dual-guide RNA pairing, Up3 +Down5
  source 1..516: mol_type = other DNA
ccggggaaaaccccgttaactccagtgggccaactgagaacggccaggatatttttcccttgagaatgactatagtttcatccgtttgaccgggacgaaaatctatccacgcaaacatggaacctgatattctgacagccgtctcttccttttgtccttcctttctttctcctctaaccaattgtttgttgtacttatgcattcatctcttcaagatgtttacttagtacctgcatacattgtagatattttcaccataatgatccatgtaaattttatgagaaacagttttcctttacattctgccttcctccattgtttctttctctctcttaggaatactttgtttattttcctctctattttttccatattgatttgaactatcaatatgacttttaatatcctctaagcagaaatgcacgcatgacttgcgaatactggtccctgataaccatccacccagatcaaagtgacctctgagaaccccccagagatttcaatcctagttggtgc

SEQ ID NO: 74 - DNA, 380 bp, synthetic construct
  misc_feature 1..380: note = Description of sequence: PCR Products from Cas9-stable HEK cells transfected with dual-guide RNA pairing, Up4 +Down4
  source 1..380: mol_type = other DNA
aaagggtggtgagtctttcgttgagttgtaattcaacactacattttgagctgaaactgaatggaaaagttggatgaggagcttcattcattgttttaccactttataagcactgtttttttttgccaccaaaaatagatgtggccaaccttccggtagaccatattacacaaggcatggcatgtgttttttaatacctctgtgctagaggataatttacaaaataaaggcacaagggctatgatagccctaaactatacagtaagcatttatttgtcaccacaggtataattcatctatgacaggttattaatggaattggacagaaaatgtttacaagtatttacattatatgagagtctggctatgtttggaagcaa

SEQ ID NO: 75 - DNA, 501 bp, synthetic construct
  misc_feature 1..501: note = Description of sequence: PCR Products from Cas9-stable HEK cells transfected with dual-guide RNA pairing, Up5 + Down1
  source 1..501: mol_type = other DNA
caatgctctgtacttggcacttaatcaaatgcatgttaaattctactgtgggtcaggtaccatgcaggatgcttggccatacaaaaataaataagataggatctcataaggcaataagcacacaattatgctacagtttgataggtgggacaacagaagagtgcacgatgagctctgcctccgactttcttgttgagatgtcttcgaaaagccacaccactggacaaagcaatatgcctggtcactgctgaggtcaacagggagaaaggacatggtcttcctgcatgaaggacatgaggacatttggtaaaacgtgataatctacaacgggaatcatatttttaagttaggttttcactatttctttttcatgtctggaatggcttcattaacccatctttccatgcttcactttgttccagactttaagtggcctccagaatgttttcgggggggggcaagtctgattatttctttaccttcaactaaagccttcaaaaa

SEQ ID NO: 76 - DNA, 506 bp, synthetic construct
  misc_feature 1..506: note = Description of sequence: PCR Products from Cas9-stable HEK cells transfected with dual-guide RNA pairing, Up5 + Down4
  source 1..506: mol_type = other DNA
ccgggtttgagtctttcgttgagttgtaattcaacactacattttgaggctgaaactgaatggaaaagttggatgagcgagcttcattcattgttttaccactttataagcactgtttttttttgccaccaaaaatagatgtggccaacccactggacaaagcaatatgcctggtcactgctgaggtcaacagggaaaaaggacatggtcttcctgcatgaaggacatgaggacatttggaaaaacgtgataatctacaacgggaatcaaatttttaagttaggttttcactatttgtttttcatgtctggaatggcttcattaacccttctttccatgcttcactttgttccaaactttaagtggcctccagaatgttttctggggggggcaagtctgattatttctttaccttgaactaaagccttcaaaaacttcccattctgtgtatgaaatgcctaaactcctaaacatggtccctgcgaatcttcccaatccaggctccc

SEQ ID NO: 77 - DNA, 504 bp, synthetic construct
  misc_feature 1..504: note = Description of sequence: PCR Products from Cas9-stable HEK cells transfected with dual-guide RNA pairing, Up5 + Down5
  source 1..504: mol_type = other DNA
acagggaaagccagttactccagtggcaactgggaacggcaggattatttttcccttgagaatgataataattcattgtttagagggaggaaaatcttccacagaacatggacctgaaattctgacaaccgctcttccttttttcctctctttctttctccttaatttattttttgttgtacttatgcattcatctcttcaagatgtttacttagtacctgatacattgtagatattttcacattaatgatccatgtaaattttatgagaaacagttttcctttacattctgccttcctccattgtttctttctctctcttaggaatactttgtttattttcctctctattttttccatattgatttgaactatcaatatgacttttaatatcctctaagcagaaatgcacgcatgacttgcactggacaaagcaatatgcctggtcactgctgaggtcaacagggagaaaggacatggtcttcctgcatgaaggacatgagga

SEQ ID NO: 78 - DNA, 152 bp, synthetic construct
  misc_feature 1..152: note = Description of sequence: GAA FGF14 repeat region with human chromosome 13 (chr13:102,813,925-102,814,076 (hg19))
  source 1..152: mol_type = other DNA
aagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaa

SEQ ID NO: 79 - DNA, 24 bp, synthetic construct
  misc_feature 1..24: note = Description of sequence: Example of a pathogenic GAA FGF14 repeat
  source 1..24: mol_type = other DNA
gaagaagaagaagaagaagaagaa

SEQ ID NO: 80 - DNA, 66 bp, synthetic construct
  misc_feature 1..66: note = Description of sequence: Upstream guide 1; edited sample 199 to 264bp
  source 1..66: mol_type = other DNA
agcatcatttggaagcatagtgccaaccttttttattatcttctacgcgcactatattgggaacgt

SEQ ID NO: 81 - DNA, 66 bp, synthetic construct
  misc_feature 1..66: note = Description of sequence: Upstream guide 1; control sample 198 to 263bp
  source 1..66: mol_type = other DNA
agcatcatttggaagcatagtgccaaccgcctcccagttagctgcaagtttcagaaacttgttcta

SEQ ID NO: 82 - DNA, 66 bp, synthetic construct
  misc_feature 1..66: note = Description of sequence: Upstream guide 2; edited sample 146 to 211bp
  source 1..66: mol_type = other DNA
cccctcatttgtatgtaggttagtagcacccacatgccatgcctagagaaatatgggctaccggaa

SEQ ID NO: 83 - DNA, 66 bp, synthetic construct
  misc_feature 1..66: note = Description of sequence: Upstream guide 2; control sample 148 to 213bp
  source 1..66: mol_type = other DNA
cccctcatttgtatgtaggttagtagcaccgacactaagggagaaaatgaatgatggttctaattc

SEQ ID NO: 84 - DNA, 66 bp, synthetic construct
  misc_feature 1..66: note = Description of sequence: Upstream guide 3; edited sample 168 to 233bp
  source 1..66: mol_type = other DNA
ctcaattgcctttgcagccagccagatcacctcctggaaacagaggaccctcaaccccaaccaccc

SEQ ID NO: 85 - DNA, 66 bp, synthetic construct
  misc_feature 1..66: note = Description of sequence: Upstream guide 3; control sample 186 to 251bp
  source 1..66: mol_type = other DNA
ctcaattgcctttgcagccagccagatcacctcctcgaatactggtccctgataaccatccaccca

SEQ ID NO: 86 - DNA, 66 bp, synthetic construct
  misc_feature 1..66: note = Description of sequence: Upstream guide 4; edited sample 162 to 277bp
  source 1..66: mol_type = other DNA
acatgccatgccttgtgtaatatggtctaccggaaagaggggttttcctacttctcttccatagag

SEQ ID NO: 87 - DNA, 66 bp, synthetic construct
  misc_feature 1..66: note = Description of sequence: Upstream guide 4; control sample 163 to 228bp
  source 1..66: mol_type = other DNA
acatgccatgccttgtgtaatatggtctaccggaagaggggttttcctacttctcttccatagagg

SEQ ID NO: 88 - DNA, 66 bp, synthetic construct
  misc_feature 1..66: note = Description of sequence: Upstream guide 5; edited sample 306 to 371bp
  source 1..66: mol_type = other DNA
ctcagcagtgaccaggcatattgctttgtccagtgtcagttccatgtttcagaagatatttgatga

SEQ ID NO: 89 - DNA, 66 bp, synthetic construct
  misc_feature 1..66: note = Description of sequence: Upstream guide 5; control sample 308 to 373bp
  source 1..66: mol_type = other DNA
ctcagcagtgaccaggcatattgctttgtccagtgtaatgtcacttacaggtagaagttttcagag

SEQ ID NO: 90 - DNA, 66 bp, synthetic construct
  misc_feature 1..66: note = Description of sequence: Downstream guide 1; edited sample 180 to 245bp
  source 1..66: mol_type = other DNA
gactttcttgttgagatgtcttcgaaaagccacacctggaggtgtggttttctgaaagcccattta

SEQ ID NO: 91 - DNA, 66 bp, synthetic construct
  misc_feature 1..66: note = Description of sequence: Downstream guide 1; control sample 180 to 245bp
  source 1..66: mol_type = other DNA
gactttcttgttgagatgtcttcgaaaagccacactggaggtgtggttttctgaaagcccatttaa

SEQ ID NO: 92 - DNA, 66 bp, synthetic construct
  misc_feature 1..66: note = Description of sequence: Downstream guide 2; edited sample 155 to 220bp
  source 1..66: mol_type = other DNA
gacaacagaagagtgcacgatgagctctgcctccgaactttcttgttgagatgtcttcgaaaagcc

SEQ ID NO: 93 - DNA, 66 bp, synthetic construct
  misc_feature 1..66: note = Description of sequence: Downstream guide 2; control sample 146 to 211bp
  source 1..66: mol_type = other DNA
gacaacagaagagtgcacgatgagctctgcctccgactttcttgttgagatgtcttcgaaaagcca

SEQ ID NO: 94 - DNA, 66 bp, synthetic construct
  misc_feature 1..66: note = Description of sequence: Downstream guide 3; edited sample 171 to 236bp
  source 1..66: mol_type = other DNA
cgtacccttatttccttcccttcttagagccccccatattgggacgtaggaaggggggaagggaga

SEQ ID NO: 95 - DNA, 66 bp, synthetic construct
  misc_feature 1..66: note = Description of sequence: Downstream guide 3; control sample 72 to 137bp
  source 1..66: mol_type = other DNA
cgtacccttatttccttcccttcttagagccccccatattggagcctaggagagtgagatggagga

SEQ ID NO: 96 - DNA, 66 bp, synthetic construct
  misc_feature 1..66: note = Description of sequence: Downstream guide 4; edited sample 112 to 177bp
  source 1..66: mol_type = other DNA
tttttttttgccaccaaaaatagatgtggccaaccgtttttcttctcccttcccccccaaccccga

SEQ ID NO: 97 - DNA, 66 bp, synthetic construct
  misc_feature 1..66: note = Description of sequence: Downstream guide 4; control sample 114 to 179bp
  source 1..66: mol_type = other DNA
tttttttttgccaccaaaaatagatgtggccaaccgtgagcattcagtcttcttctccaagcccta

SEQ ID NO: 98 - DNA, 66 bp, synthetic construct
  misc_feature 1..66: note = Description of sequence: Downstream guide 5; edited sample 388 to 453bp
  source 1..66: mol_type = other DNA
taatatcctctaagcagaaatgcacgcatgacttggaagagtatttaaaaagtagagggggggtgg

SEQ ID NO: 99 - DNA, 66 bp, synthetic construct
  misc_feature 1..66: note = Description of sequence: Downstream guide 5; control sample 387 to 452bp
  source 1..66: mol_type = other DNA
taatatcctctaagcagaaatgcacgcatgacttgaagtggctttgaagactaactgaagtgctga

SEQ ID NO: 100 - DNA, 1550 bp, synthetic construct
  misc_feature 1..1550: note = Description of sequence: Disease haplotype with GAA repeat locus
  source 1..1550: mol_type = other DNA
  variation 24..1520: note = position may be absent
  misc_feature 21..1520: note = GAA repeat subunit may be repeated from 1 to 500 times
tgttggtggactagcactaggaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaatgagcattctgacagctcttcccaactcgg

SEQ ID NO: 101 - DNA, 1550 bp, synthetic construct
  misc_feature 1..1550: note = Description of sequence: Disease haplotype with GAA repeat locus
  source 1..1550: mol_type = other DNA
  variation 24..1520: note = position may be absent
  misc_feature 21..1520: note = GAA repeat subunit may be repeated from 1 to 500 times
tgttggtggactagcactaggaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaatgagcattctgacagccctttccagctcgg

SEQ ID NO: 102 - DNA, 1550 bp, synthetic construct
  misc_feature 1..1550: note = Description of sequence: Disease haplotype with GAA repeat locus
  source 1..1550: mol_type = other DNA
  variation 24..1520: note = position may be absent
  misc_feature 21..1520: note = GAA repeat subunit may be repeated from 1 to 500 times
tgttggtggactagcactaggaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaatgagcgtccgggtagccctttccagctcgg

SEQ ID NO: 103 - DNA, 1550 bp, synthetic construct
  misc_feature 1..1550: note = Description of sequence: Disease haplotype with GAA repeat locus
  source 1..1550: mol_type = other DNA
  variation 24..1520: note = position may be absent
  misc_feature 21..1520: note = GAA repeat subunit may be repeated from 1 to 500 times
tgttggtggactagcactaggaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaatgagcgtccgggtagccctttccaactcgg

(19) United States
(12) Patent Application Publication
(10) Pub. No.: US 2026/0209754 A1
(43) Pub. Date: Jul. 23, 2026

(54) GENE EDITING SYSTEMS FOR TREATING AND PREVENTING FGF14 GAA CEREBELLAR ATAXIAS

(71) Applicant: UNIVERSITY OF MIAMI, Miami, FL (US)

(72) Inventors: Stephan L. ZUCHNER, Miami, FL (US); Adriana REBELO, Miami, FL (US)

(21) Appl. No.: 19/138,988

(22) PCT Filed: Dec. 14, 2023
(86) PCT No.: PCT/US2023/084052

§ 371 Date: Jun. 13, 2025

Related U.S. Application Data
(60) Provisional application No. 63/387,329, filed on Dec. 14, 2022.

(51) Int. Cl. C12N 15/11; C12N 9/22; C12N 15/90

(52) U.S. Cl. CPC C12N 15/11; C12N 9/226; C12N 15/907; C12N 2310/20

(57) ABSTRACT

The present disclosure relates to gene editing systems and methods for treating and preventing cerebellar ataxias caused by FGF14 GAA repeat expansions.

### RELATED APPLICATION

This PCT application claims priority to, and the benefit of, U.S. Provisional Patent Application No. 63/387,329, filed Dec. 14, 2022, which is incorporated by reference herein in its entirety.

### STATEMENT REGARDING FEDERALLY SPONSORED RESEARCH

This invention was made with Government Support under Grant No. R01NS072248 awarded by the National Institutes of Health. The Government has certain rights in the invention.

### REFERENCE TO SEQUENCE LISTING

The sequence listing submitted on Dec. 14, 2023, as an .XML file entitled “11348-045WO1_ST26.xml” created on Dec. 13, 2023, and having a file size of 156,673 bytes is hereby incorporated by reference pursuant to 37 C.F.R. § 1.52 (e) (5).

### FIELD

The present disclosure relates to gene editing systems and methods for treating and preventing cerebellar ataxias caused by FGF14 GAA repeat expansions.

### BACKGROUND

Late-onset cerebellar ataxias (LOCAs) are a heterogeneous group of neurodegenerative disorders manifesting as a progressive cerebellar syndrome that develops after 30 years of age. The prevalence of LOCA is approximately 1 to 3 per 100,000 population, and molecular testing yields negative results in almost 75% of patients with LOCA. This is explained in part by the limitations of standard next-generation sequencing analysis for the identification of certain sequence variations, such as tandem repeat expansions.

Given limitations of diagnosing and treating late-onset cerebellar ataxia, there is need to address the aforementioned problems mentioned above by developing systems and methods to edit genetic sequences causing LOCA and other forms of ataxia. The systems and methods disclosed herein address these and other needs.

### SUMMARY

The present disclosure provides a gene editing system targeting trinucleotide repeats within an intronic region of the fibroblast growth factor 14 gene. The present disclosure also provides a method of gene editing an FGF14 gene in a cell or subject. The present disclosure thus provides methods of treating or preventing late-onset cerebellar ataxia.

In one aspect, disclosed herein is a gene editing system comprising a first guide RNA (gRNA) or a first nucleic acid sequence encoding the first gRNA, a second gRNA or a second nucleic acid sequence encoding the second gRNA, and an endonuclease or a third nucleic acid sequence encoding the endonuclease; wherein the first and second gRNAs target the endonuclease to a first and a second genomic loci in a nucleus of a cell that together flank a GAA repeat in intron 1 of a fibroblast growth factor 14 (FGF14) gene.

In some embodiments, the gene editing system is a CRISPR gene editing system. In other embodiments, the endonuclease is a Cas9 or Cpf1 endonuclease.

In some embodiments, the first gRNA comprises SEQ ID NO: 1, SEQ ID NO: 2, SEQ ID NO: 3, SEQ ID NO: 4, or SEQ ID NO: 5. In other embodiments, the second gRNA comprises SEQ ID NO: 6, SEQ ID NO: 7, SEQ ID NO: 8, SEQ ID NO: 9, or SEQ ID NO: 10. In other embodiments, the first and second genomic loci are upstream (5′) of the GAA repeat and downstream (3′) of the GAA repeat.

In some embodiments, the gene editing system removes all GAA repeats. In some embodiments, the gene editing system removes at least 250 GAA repeats. In other embodiments, the gene editing system removes at least 300 GAA repeats.

In some embodiments, the gene editing system is administered with a pharmaceutically acceptable carrier, salt, solvent, or excipient.

In one aspect, disclosed herein is a method of gene editing of a fibroblast growth factor 14 (FGF14) gene in a cell or a subject, the method comprising targeting a first guide RNA (gRNA) and an endonuclease to a first genomic loci of the FGF14 gene, targeting a second gRNA and the endonuclease to a second genomic loci of the FGF14 gene, and removing a GAA repeat in intron 1 of the FGF14 gene.

In some embodiments, the method comprises a CRISPR gene editing system. In other embodiments, the endonuclease is a Cas9 or Cpf1 endonuclease.

In some embodiments, the first gRNA comprises SEQ ID NO: 1, SEQ ID NO: 2, SEQ ID NO: 3, SEQ ID NO: 4, or SEQ ID NO: 5. In other embodiments, the second gRNA comprises SEQ ID NO: 6, SEQ ID NO: 7, SEQ ID NO: 8, SEQ ID NO: 9, or SEQ ID NO: 10.

In some embodiments, the method removes at least 250 GAA repeats. In other embodiments, the method removes at least 300 GAA repeats. In other embodiments, the first and second genomic loci are upstream (5′) of the GAA repeat and downstream (3′) of the GAA repeat.

In some embodiments, the method treats or prevents a cerebellar, a spinocerebellar, or an episodic ataxia. In other embodiments, the subject is a human.

In another aspect, disclosed herein is a method of treating or preventing a late-onset cerebellar ataxia (LOCA), the method comprising administering to a subject in need thereof a gene editing system to at least one cell in the subject, wherein the gene editing system comprises a first guide RNA (gRNA) or a first nucleic acid sequence encoding the first gRNA a second gRNA or a second nucleic acid sequence encoding the second gRNA and an endonuclease or a third nucleic acid sequence encoding the endonuclease wherein the first and second gRNAs target the endonuclease to a first and a second genomic loci in a nucleus of the cell that together flank a GAA repeat in intron 1 of a fibroblast growth factor 14 (FGF14) gene.

In one embodiment, the method comprises a CRISPR gene editing system. In one embodiment, the method comprises a CRISPR/Cas gene editing system. In other embodiments, the endonuclease is a Cas9 or a Cpf1 endonuclease.

In some embodiments, the first gRNA comprises SEQ ID NO: 1, SEQ ID NO: 2, SEQ ID NO: 3, SEQ ID NO: 4, or SEQ ID NO: 5. In other embodiments, the second gRNA comprises SEQ ID NO: 6, SEQ ID NO: 7, SEQ ID NO: 8, SEQ ID NO: 9, or SEQ ID NO: 10.

In some embodiments, the endonuclease partially or completely removes the GAA repeat at intron 1 of the FGF14 gene. In other embodiments, the FGF14 gene is reassembled by a recombination event. In other embodiments, the gene editing system removes at least 250 GAA repeats. In other embodiments, the gene editing system removes at least 300 GAA repeats.

In some embodiments, the first and second genomic loci are upstream (5′) of the GAA repeat and downstream (3′) of the GAA repeat. In other embodiments, the gene editing system is administered with a pharmaceutically acceptable carrier, salt, solvent, or excipient. In other embodiments, the subject is a human.

### BRIEF DESCRIPTION OF FIGURES

The accompanying figures, which are incorporated in and constitute a part of this specification, illustrate several aspects described below.

FIGS. 1A, 1B, 1C, 1D, 1E, and 1F show the identification of a Deep Intronic GAA Repeat Expansion in FGF14 in Patients with Late-Onset Cerebellar Ataxia. FIG. 1A shows the pedigrees of three large unrelated French Canadian families with unsolved autosomal dominant late-onset cerebellar ataxia (LOCA). Numbered family members underwent genotyping for the GAA repeat expansion in FGF14 by long-range polymerase chain reaction (PCR) and repeat-primed PCR. Allele sizes expressed as numbers of GAA repeats are provided for clinically affected persons only. Family members for whom whole-genome sequences were obtained are indicated by a red box. Squares represent male family members, and circles female family members. Solid black shapes indicate affected persons. Question marks indicate persons whose clinical status is uncertain. Slashed symbols indicate deceased persons. Probands are indicated by arrows. Pedigrees have been randomized and abbreviated to preserve privacy. FIG. 1B shows a diagram of FGF14 transcript variants 1 (NM_004115.4) and 2 (NM_175929.3). The location of the (GAA) n repeat locus in the first intron of FGF14 transcript variant 2 is indicated by the red arrowhead. FIG. 1C shows the distribution of anchored in-repeat reads (IRRs) for each of the 2504 samples from the 1000 Genomes Project control cohort, 1115 samples from the Vanderbilt Atrial Fibrillation Registry (VAFR) control cohort, and 6 French Canadian patients with LOCA at the FGF14 repeat locus (chr13:102,813,925-102,814,074; GRCh37). The mean (±SD) number of anchored IRRs at this locus was 16.68±1.90 (median, 16.40; range, 14.60 to 19.45) in the group of 6 patients with LOCA, which was higher than that in the 1000 Genomes Project data set (1.77±4.99; median, 0; range, 0 to 40.42; Cohen's d, 2.99; 95% confidence interval [CI], 2.19 to 3.80]) and the VAFR cohort (4.38±7.73; median, 0; range, 0 to 50.78; Cohen's d, 1.59; 95% CI, 0.78 to 2.40). The widths of the confidence intervals have not been adjusted for multiplicity, and therefore the confidence intervals should not be used to reject or not reject effects. FIG. 1D shows the results of long-range PCR demonstrating a large heterozygous expansion of the FGF14 repeat locus. A representative image of PCR amplification products that were resolved with the use of the Agilent 4200 TapeStation automated electrophoresis system from two controls and four patients is shown. All samples were amplified and resolved during the same experiment. The four patients each carried one expanded product that was at least 900 bp in length, corresponding to 250 or more GAA repeats. The large amplification product of Control FC-C57 was found by long-read nanopore sequencing to be a GAAGGA hexanucleotide repeat expansion. MW denotes molecular weight. FIGS. 1E and 1F show the results of repeat-primed PCR of the FGF14 GAA repeat unit. FIG. 1E shows a normal electropherogram in a control who was homozygous for (GAA) 11 alleles. FIG. 1F shows an electropherogram showing the characteristic sawtooth pattern in a patient with LOCA (Patient II.6) carrying an expanded (GAA) 550 allele.

FIGS. 2A, 2B, 2C, 2D, and 2E show the allele distribution and long-read sequencing of the FGF14 GAA repeat locus. FIGS. 2A and 2B show allele frequencies of the FGF14 repeat locus in 408 controls (816 chromosomes) (FIG. 2A) and 128 patients with GAA-FGF14-related ataxia (122 normal and 134 expanded chromosomes) (FIG. 2B). The repeat length was estimated by agarose gel electrophoresis of PCR-amplification products. Among the patients with GAA-FGF14-related ataxia, 4 were homozygous or compound heterozygous for (GAA)_{≥}250 expansions, and 2 were compound heterozygous for a (GAA) 250 expansion and a (GAAGGA)_{≥}125 expansion. The box-and-whisker plots show the allelic distribution in controls and patients. In FIG. 2B, the relative frequencies of the normal and expanded alleles are shown; the box-and-whisker plots above the graph show the distribution of the normal alleles (left-hand plot) and expanded alleles (right-hand plot) in patients. The box indicates the 25th percentile (first quartile), the median, and the 75th percentile (third quartile), and the whiskers indicate the 2.5th and 97.5th percentiles. Outliers are represented by black dots. In controls, expanded alleles consisting of non-GAA repeats are represented by red triangles. The dashed gray lines and the shaded gray areas indicate the incompletely penetrant range of (GAA) 250-300, and the dashed red lines mark the threshold of (GAA) 300 repeat units, above which the alleles are fully penetrant. The allelic distributions in the different control and patient cohorts are shown in FIG. 17. FIG. 2C shows swarm plots of 3000 randomly sampled individual nanopore reads containing at least 50 repeat units of 2 controls and 6 patients. Control G-C164 carries a subpathogenic (GAA)₂₂₂ allele, and control FC-C57 carries a nonpathogenic (GAAGGA) 152 allele. Patients I.22, II.6, and III.3 (French Canadian discovery cohort) carry a (GAA)₃₈₈, (GAA)₅₀₈, and (GAA)₃₁₉ expansion, respectively. Patients G6 (German cohort) and A2 (Australian cohort) carry a (GAA)₃₄₅ and (GAA)₄₃₇ expansion, respectively. Patient FC2 (French Canadian cohort) carries a (GAA)₂₂₃ allele and a (GAA)₃₅₅ allele. Horizontal black bars indicate repeat size of the larger allele, as measured by nanopore sequencing. Expansion sizing by long read nanopore sequencing and agarose gel electrophoresis of PCR amplification products is highly similar (Pearson's correlation coefficient, 0.96). The horizontal dashed gray line and the shaded gray area show the incompletely penetrant range of (GAA)₂₅₀₋₃₀₀, and the dashed red line marks the threshold of (GAA)₃₀₀ repeat units, above which the alleles are fully penetrant. The color of the data points is a function of the GAA repeat motif purity in each individual read, with dark blue indicating pure and lighter blue impure motif (a hue scale is shown on the right y axis). FIG. 2D shows the percentages of patients with LOCA who carried an FGF14 (GAA)_{≥}250 repeat expansion in the French Canadian (40 of 66 index patients), German (42 of 228), Australian (3 of 20), and Indian (3 of 31) cohorts. FIG. 2E shows repeat-length variation across 15 maternal and 15 paternal meiotic events involving alleles of (GAA)_{≥}250 repeats.

FIGS. 3A, 3B, 3C, 3D, 3E, 3F, 3G, 3H, 3I, and 3J show the imaging and neuropathological findings in patients with GAA-FGF14-related ataxia. FIG. 3A shows severe vermis atrophy (arrowhead) on sagittal T1-weighted magnetic resonance imaging in a female patient at 88 years of age. FIG. 3B shows a midsagittal section of the postmortem cerebellum of the same patient at 94 years of age, in which anterior vermis atrophy is visible (arrowhead); FIG. 3C shows an age-matched control for comparison. FIG. 3D shows a hematoxylin and eosin-stained section of the cerebellar vermis in the patient, and a control is shown for comparison in FIG. 3E. Widespread loss of Purkinje cells, with shrunken appearance of rare residual Purkinje cells (arrow), a gliotic and rarefied molecular layer (black asterisk), and reduced numbers of cells in the granule-cell layer (blue asterisk) can be seen in the patient. FIGS. 3F and 3H show calbindin immunohistochemical analysis of the vermis from the same patient with GAA-FGF14-related ataxia and a control, respectively. Severe loss of Purkinje neurons with markedly rarefied dendritic network in the molecular layer is seen in the patient, whereas in the age-matched control, the Purkinje cells show dense dendritic arborization in the molecular layer. In the inset shown in FIG. 3G, Purkinje-cell loss is also evident with Bielschowsky tinctorial silver stain, which highlights the processes of basket cells, resulting in an “empty basket” appearance. FIGS. 3I and 3J show the cerebellar hemisphere of a patient with GAA-FGF14-related ataxia and a control, respectively; in the patient, the Purkinje cells are reduced in number (arrows), but the molecular layer shows less prominent gliosis and the cell density in the granule-cell layer is much better preserved than in the vermis. All staining was performed with appropriate negative and positive controls. Pathological findings in the cerebellum were similar in the two French Canadian patients with GAA-FGF14-related ataxia for whom postmortem tissue was available. The scale bar in FIGS. 3D-3F and Panels FIGS. 3H-3J indicates 100 μm, and the scale bar in FIG. 3G indicates 60 μm.

FIGS. 4A, 4B, 4C, 4D, 4E, 4F, and 4G show the FGF14 expression and protein levels in cerebellum and iPSC-derived motor neurons. FIG. 4A shows relative expression of both FGF14 transcripts, transcript variant 1 (NM_004115.4) and transcript variant 2 (NM_175929.3) mRNA in the postmortem cerebellar cortex of six controls and two patients, normalized by geometric averaging of the expression of five housekeeping genes (ACTB, HPRT1, YWHAZ, RPL13, and UBE2D2) as assessed by quantitative PCR. Values shown are the ratio of the mean expression relative to the mean among controls. Bars indicate the mean, T bars the standard deviation, and black dots the data distribution. FIG. 4B shows a map of the primers used in quantitative PCR experiments with postmortem cerebellum and induced pluripotent stem cell (iPSC)—derived motor neurons in relation to the exons of both FGF14 transcripts. Orange arrows indicate the primers used for the transcript 1 assay, dark purple arrows the primers used for the transcript 2 assay, and green arrows and light purple arrows the primers used for total FGF14 expression assays in postmortem cerebellum and iPSC-derived motor neurons, respectively. The position of the GAA repeat expansion in intron 1 of transcript 2 is indicated by a dark purple arrowhead. FIG. 4C shows a representative FGF14 immunoblot of protein extracts from postmortem cerebellar cortex specimens from seven controls and two patients. Beta-tubulin was used as loading control. Western blot analysis was repeated independently three times, with similar results. FIG. 4D shows the mean expression ratios of FGF14 protein in postmortem cerebellar specimens from seven controls and two patients, measured across three independent replicate immunoblots. All ratios were normalized to beta-tubulin and expressed relative to controls. Bars indicate the mean, T bars the standard deviation, and black dots the data distribution. FIG. 4E shows the relative expression of FGF14 transcript 2 in iPSC-derived motor neurons of two controls and two patients, normalized to GAPDH, as assessed by quantitative PCR. Relative quantification was computed by the 2^{-ΔΔct} method, with the use of the mean value among controls as calibrator; values are represented as the ratio of the mean expression relative to the mean among controls. Bars indicate the mean, and black dots and triangle the data distribution. FIG. 4F shows a representative FGF14 immunoblot of protein extracts from induced motor neurons from two controls and two patients. GAPDH was used as loading control. Western blot analysis was repeated independently twice, with similar results. FIG. 4G shows the mean expression ratios of FGF14 protein in induced motor neurons from two controls and two patients. All ratios were normalized to GAPDH and expressed relative to controls. Bars indicate the mean, and black dots and triangle the data distribution. Black triangles in FIGS. 4E and 4G indicate a patient who was homozygous for (GAA)₃₀₀ expansions.

FIGS. 5A and 5B show the FGF14 repeat region: chr13:102,813,925-102,814,076 (hg19). FGF14 gene is located on the minus strand and consists of 5 exons and 4 introns. It is approximately 680,920 base pairs long and encodes for 252 amino acids. FGF14 contains a GAA trinucleotide repeat located within Intron 1.

FIG. 6 shows the FGF14 GAA trinucleotide repeat expansion. The expansion of nucleotides chosen are guide RNAs within a region 20,000 base pairs upstream and downstream of the GAA trinucleotide repeat expansion. This distance was chosen due to the large size of Intron 1, spanning 526,174 base pairs total. Guides designed within this region do not pose a risk to cutting coding regions near Exon 1 or Exon 2.

FIGS. 7A, 7B, 7C, 7D, and 7E show the sequencing results of upstream gRNAs (Upstream guides 1-5).

FIGS. 8A, 8B, 8C, 8D, and 8E show the sequencing results of downstream gRNAs (Downstream guides 1-5).

FIGS. 9A and 9B shows electrophoresis gels of 16 paired gRNAs. The Cas9-stable HEK cells were transfected with various dual-guide pairings. The DNA from these were then sequenced, and the PCR products were run on a gel. The presence of bands is unexpected because the region being sequenced is too large for a standard PCR, therefore seeing bands on the gel indicates that some region must have been excised and is now small enough for a successful PCR product to be made.

FIGS. 10A, 10B, 10C, 10D, 10E, 10F, 10G, 10H, 10I, 10J, 10K, and 10L show the sequencing results of pairing upstream and downstream gRNAs.

FIGS. 11A,11B, 11C, 11D, 11E, 11F, 11G, 11H, 11I, 11J, 11K, 11L, and 11M show the excised regions of GAA trinucleotides within human FGF14 gene using UCSC Genome Browser.

FIG. 12 shows the primer target sites on human FGF14 gene using primers for sequencing within all the cutting/editing regions and primer for sequencing outside of the cutting/editing regions.

FIG. 13 shows the real-time PCR quantification after excision of trinucleotide regions using combinations of gRNAs.

FIG. 14 shows the average real-time PCR quantification after excision of trinucleotide regions using combinations of gRNAs.

FIG. 15 shows the REViewer visualization of the heterozygous GAA repeat expansion in FGF14. Alignment of short-reads to the FGF14 intronic locus (chr13: 102,813,925-102,814,074; GRCh37) shows the presence of a heterozygous GAA repeat expansion in all six French-Canadian LOCA cases who underwent whole-genome sequencing. A representative image from patient 1.8 (see FIG. 1A) is shown.

FIG. 16 shows the long-range PCR shows a large heterozygous expansion of the FGF14 repeat locus in late-onset cerebellar ataxia cases compared to controls. Long-range PCR results of two controls (C) and the six late-onset cerebellar ataxia (LOCA) cases (P) who were found by analysis of whole-genome sequencing to have a large heterozygous expansion at the FGF14 repeat locus. See FIG. 1A, red boxes.

FIGS. 17A, 17B, 17C, and 17D show the allelic distribution of the FGF14 repeat locus in control and patient cohorts. Allelic frequencies of the FGF14 repeat locus in (FIG. 17A) 209 French-Canadian controls (N=418 chromosomes), (FIG. 17B) 199 German controls (N=398 chromosomes), (FIG. 17C) 73 French-Canadian GAA-FGF14-related ataxia cases (N=71 normal and N=75 expanded chromosomes) and (FIG. 17D) 55 German (n=49), Australian (n=3) and Indian (n=3) GAA-FGF14-related ataxia cases (N=51 normal and N=59 expanded chromosomes). The repeat length was estimated by agarose gel electrophoresis of PCR amplification products. The box-and-whisker plots show the allelic distribution in control and patient cohorts. In FIGS. 17C and 17D, the relative allelic frequencies of the normal and expanded alleles are shown; the left-sided and right-sided box-and-whisker plots show the distribution of the normal and expanded alleles in patients, respectively. The box indicates the 25^{th} percentile (Q1), the median and the 75^{th} percentile (Q3). The whiskers represent the 2.5^{th} and 97.5^{th} percentiles. Outliers are represented by black dots. In controls, expanded alleles consisting of an impure repeat are represented by red triangles. The dashed grey lines and shaded grey areas show the incompletely penetrant range of (GAA)₂₅₀₋₃₀₀, and the dashed red lines mark the absolute pathogenic threshold of >(GAA)₃₀₀ repeat units. The allelic distribution is not statistically different between the French-Canadian and German control cohorts (two-sample Kolmogorov-Smirnov test, p=0.41), and between the French-Canadian, and the combined German, Australian and Indian patient cohorts (two-sample Kolmogorov-Smirnov test, p=0.15).

FIG. 18 shows the nanopore sequencing of the FGF14 repeat locus in nine selected French-Canadian controls. Swarmplots showing 3,000 randomly sampled individual nanopore reads containing at least 50 repeat units of nine selected French-Canadian controls. Samples are presented in a waterfall arrangement in descending order of allele size. Horizontal black bars indicate repeat size of the larger allele as estimated by a Gaussian mixture model. The horizontal dashed grey line and the shaded grey area show the incompletely penetrant range of (GAA)₂₅₀₋₃₀₀, and the dashed red line marks the absolute pathogenic threshold of >(GAA)₃₀₀ repeat units. Dot color is a function of individual read's repeat motif purity, with dark blue indicating pure and lighter blue impure motif (hue scale on right y axis). Controls FC-C57 and FC-C85 carried a (GAAGGA) n expansion while control FC-C94 carried a [(GAA)₄ (GCA) 1]ₙ expansion. None of the GAA pure expansions exceeded 300 repeat units in controls.

FIG. 19 shows the nanopore sequencing of the FGF14 repeat locus in seven selected German controls with large alleles. Swarmplots showing 3,000 randomly sampled individual nanopore reads containing at least 50 repeat units of seven selected German controls carrying a large allele. Samples are presented in a waterfall arrangement in descending order of allele size. Horizontal black bars indicate repeat size of the larger allele as estimated by a Gaussian mixture model. The horizontal dashed grey line and the shaded grey area show the incompletely penetrant range of (GAA)₂₅₀₋₃₀₀, and the dashed red line marks the absolute pathogenic threshold of >(GAA)₃₀₀ repeat units. Dot color is a function of individual read's GAA repeat motif purity, with dark blue indicating pure and lighter blue impure motif (hue scale on right y axis). Controls G-C124 and G-C164 carried an allele <250 GAA repeats as estimated by long-range PCR and nanopore sequencing. None of the GAA pure expansions exceeded 300 repeat units in controls.

FIGS. 20A and 20B shows the polymorphic configurations of the FGF14 repeat locus. Allele distribution of sequence polymorphisms at the FGF14 locus in (FIG. 20A) 311 of 2,504 samples (12.42%) of the 1000 Genomes Project cohort and (FIG. 20B) 301 of 1,115 samples (27.00%) of the Vanderbilt Atrial Fibrillation Registry (VAFR) cohort with evidence of at least five anchored IRRs at the locus, supportive of an approximately >58 trinucleotide-long repeat.

FIG. 21 shows the comparison of FGF14 expansion sizing by long-range PCR and nanopore sequencing. Expansion sizing of 131 alleles by agarose gel electrophoresis and nanopore is highly similar (Pearson's correlation coefficient 0.96, p<0.0001). Nanopore sizing was performed on size-selected (>400 bp) PCR amplification products only; the graph shows data for alleles >80 repeat units (>400 bp) only. Amplification products obtained through two separate and independent PCR experiments were used for nanopore and agarose gel electrophoresis sizing. The dashed grey lines and the shaded grey area show the incompletely penetrant range of (GAA)₂₅₀₋₃₀₀, and the dashed red lines mark the absolute pathogenic threshold of >(GAA)₃₀₀ repeat units. Long-range PCR was used as the standard technique to establish repeat size for each individual sample.

FIG. 22 shows the long-range PCR yields stable repeat expansion size in triplicate experiments. Long-range PCR assay allows for accurate sizing of large FGF14 repeat expansions. The size of the repeat expansion in three LOCA cases is stable across triplicate experiments. LOCA2 is homozygous for an expanded (GAA)₃₀₀ allele.

FIG. 23 shows the nanopore sequencing of the FGF14 repeat locus in 55 French-Canadian GAA-FGF14-related ataxia cases. Swarm plots showing 3,000 randomly sampled individual nanopore reads containing at least 200 repeat units of 55 French-Canadian GAA-FGF14-related ataxia cases. Samples are presented in a waterfall arrangement in descending order of allele size. Horizontal black bars indicate repeat size of the larger allele as estimated by a Gaussian mixture model. The horizontal dashed grey lines and shaded grey areas show the incompletely penetrant range of (GAA)₂₅₀₋₃₀₀, and the dashed red lines mark the absolute pathogenic threshold of >(GAA)₃₀₀ repeat units. Dot color is a function of individual read's GAA repeat motif purity, with dark blue indicating pure and lighter blue impure motif (hue scale on right y axis).

FIG. 24 shows the nanopore sequencing of the FGF14 repeat locus in 43 German GAA-FGF14-related ataxia cases. Swarmplots showing 3,000 randomly sampled individual nanopore reads containing at least 200 repeat units of 43 German GAA-FGF14-related ataxia cases. Samples are presented in a waterfall arrangement in descending order of allele size. Horizontal black bars indicate repeat size of the larger allele as estimated by a Gaussian mixture model. The horizontal dashed grey lines and shaded grey areas show the incompletely penetrant range of (GAA)₂₅₀₋₃₀₀, and the dashed red lines mark the absolute pathogenic threshold of >(GAA)₃₀₀ repeat units. Dot color is a function of individual read's GAA repeat motif purity, with dark blue indicating pure and lighter blue impure motif (hue scale on right y axis).

FIG. 25 shows the nanopore sequencing of the FGF14 repeat locus in three Australian GAA-FGF14-related ataxia cases. Swarmplots showing 3,000 randomly sampled individual nanopore reads containing at least 200 repeat units of three Australian GAA-FGF14-related ataxia cases. Samples are presented in a waterfall arrangement in descending order of allele size. Horizontal black bars indicate repeat size of the larger allele as estimated by a Gaussian mixture model. The horizontal dashed grey line and the shaded grey area show the incompletely penetrant range of (GAA)₂₅₀₋₃₀₀, and the dashed red line marks the absolute pathogenic threshold of >(GAA)₃₀₀ repeat units. Dot color is a function of individual read's GAA repeat motif purity, with dark blue indicating pure and lighter blue impure motif (hue scale on right y axis).

FIG. 26 shows the nanopore sequencing of the FGF 14 repeat locus in three LOCA (non-GAA-FGF14-related ataxia) cases shows a large non GAA pure expansion. Swarmplots showing 3,000 randomly sampled individual nanopore reads containing at least 100 repeat units of three late-onset cerebellar ataxia (non-GAA-FGF14-related ataxia) cases found to carry a large non-pathogenic (GAAGGA) n hexanucleotide expansion. Samples are presented in a waterfall arrangement in descending order of allele size. Horizontal black bars indicate repeat size of the larger allele as estimated by a Gaussian mixture model.

FIGS. 27A, 27B, and 27C show the repeat-primed PCR analysis of subjects carrying a GAA and GAAGGA repeat expansion. Representative plots of the repeat-primed PCR (RP-PCR) targeting the GAA repeat unit in (FIG. 27A) a GAA-FGF14-related ataxia case carrying a GAA repeat expansion, (FIG. 27B) a control (Control FC-C57, see FIG. 1D and FIG. 2C) carrying a GAAGGA repeat expansion and (FIG. 27C) a control homozygous for (GAA)₁₁ alleles.

FIG. 28 shows ancestry classification of 14 GAA-FGF14-related ataxia cases. Scatterplot of the first two principal components (PC) of an ancestry analysis. The 1000 Genomes Project cohort samples are plotted as small dots, colored according to their stated ancestry. Fourteen individuals carrying the FGF14 GAA expansion are projected onto the PC axes and plotted as squares. They are all colored purple because they were predicted to be of European origin. AFR: African; AMR: Admixed American; EAS: East Asian; EUR: European; SAS: South Asian

FIGS. 29A, 29B, 29C, 29D, and 29E show the FGF14 repeat locus allele sizes in superpopulations of the 1000 Genomes Project cohort. Histograms of the distribution of allele sizes predicted by ExpansionHunter in the 1000 Genomes Project cohort. Individuals from different superpopulations are plotted in separate histograms as follows: FIG. 29A) African (AFR), FIG. 29B) Admixed American (AMR), FIG. 29C) East Asian (EAS), FIG. 29D) European (EUR) and FIG. 29E) South Asian (SAS).

FIG. 30 shows the distribution of anchored in-repeat reads (IRR) for the European subset of the 1000 Genomes Project cohort, the VAFR cohort and six French-Canadian GAA-FGF14-related ataxia cases. The mean anchored IRR count at this locus was 16.68 (SD 1.90, median 16.40, range 14.60-19.45) in the six LOCA cases group, higher than that of the 1000 Genomes Project dataset (mean 3.67, SD 6.89, median 0, range 0-39.04; Cohen's d 1.90) and the VAFR cohort (mean 4.38, SD 7.73, median 0, range 0-50.78; Cohen's d 1.59).

FIG. 31 shows the haplotype analysis of six French Canadian and one Australian GAA-FGF14-related ataxia cases. The presence of a disease haplotype composed of 50 common polymorphic loci surrounding the GAA repeat locus was tested in seven samples. Loci containing the disease haplotype polymorphism in an individual are displayed in green, while those without the matching polymorphism are displayed in red.

FIG. 32 shows the correlation between FGF14 GAA expansion length and age at onset. Weak inverse linear correlation between GAA repeat size and age at onset of disease in 123 cases (Pearson's correlation coefficient=−0.32, R²=0.10 p=0.0004). The grey area displays the 95% confidence interval. Subjects homozygous or compound heterozygous for ≥(GAA)₂₅₀ alleles are represented by red triangles. The size of their largest expansion was used for analysis. Using aggregate repeat counts did not yield more robust correlation (Pearson's correlation coefficient=−0.24, R²=0.06, p=0.01). The age at which disease first manifested, either as episodic or permanent ataxia, was used for the analysis.

FIG. 33 shows the post-hoc genome-wide parametric linkage analysis in Family I. Following discovery of the intronic GAA expansion, two-point parametric linkage analysis using the mutation status in FGF14 as the phenotype in 18 individuals belonging to Family I (see FIG. 1A) identified significant linkage loci with logarithm of the odds (LOD) score >3 on chromosomes 12, 13, 14, and 18. The highest LOD scores were obtained on chromosome 13 at SNP rs9513827 (LOD=4.31) and rs12870187 (LOD=4.16).

FIG. 34 shows genomic regional plot of the linkage region with the highest LOD score on chromosome 13. The red dashed lines indicate the 1.4 Mb-long linkage region with the highest LOD score located between SNP markers rs9513827 (LOD=4.31) and rs12870187 (LOD=4.16) on chr13: 101,602, 162-103,083,549 (GRCh37). This region fully encompasses FGF14.

FIGS. 35A, 35B, 35C, and 35D show induced pluripotent stem cells (iPSCs) and motor neuron editing techniques. FIG. 35A shows the transfection of gRNA pair(s) into FGF14 Cas9-stable iPSCs with Lipofectamine Messenger MAX. FIG. 35B shows the transfection of gRNA pair(s) into FGF14 Cas9-stable iPSCs with Lipofectamine Messenger MAX and differentiation into motor neurons. FIG. 35C shows the differentiation of Cas9-stable iPSCs into motor neurons and transfection of gRNA pair(s) into the motor neurons with Lipofectamine Messenger MAX. FIG. 35D shows the differentiation of unstable Cas9 iPSCs into motor neurons and transfection of Cas9/gRNA ribonucleoprotein (RNP) complexes into motor neurons with Lipofectamine Messenger MAX.

FIGS. 36A, 36B, and 36C show the generation and characterization of FGF14 repeat expansion knock-in rats. FIG. 36A shows the linear plasmid vector cloning of FGF14 human repeat expansion. FIG. 36B shows the injection of FGF14 repeat expansion construct into fertilized eggs to generate knock-in rats. FIG. 36C shows the characterization of FGF14 knock-in rats and phenotype rescue with Crispr-AAV.

### DETAILED DESCRIPTION

The present disclosure provides a gene editing system targeting trinucleotide repeats within an intronic region of the fibroblast growth factor 14 (FGF14) gene. The inventors have unexpectedly identified a GAA repeat or expansion in intron 1 of the FGF14 that is associated with late-onset cerebellar ataxia (LOCA), and provide methods of gene editing an FGF14 gene in a cell or subject. The present disclosure also provides methods of treating or preventing late-onset cerebellar ataxia.

Those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “polymerase chain reaction” (“PCR”) refers to a method for increasing the concentration of a segment of a target sequence in a mixture of genomic DNA without cloning or purification. This process for amplifying the target sequence typically consists of introducing a large excess of two oligonucleotide primersto the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multiplex-PCR, reverse transcription PCR, or any of the other PCR variants known to those skilled in the art.

As used herein, the term, “deletion”, also called gene deletion, deficiency, or deletion mutation, refers to part of a chromosome or a sequence of DNA being left out during DNA replication or deleted by gene editing and excision. Deletion, or gene deletions can cause any number of nucleotides to be deleted from a single base to an entire piece of chromosome.

A “vector” refers to a composition used as a vehicle to artificially carry foreign genetic material into another cell or tissue. A vector can be a bacterial plasmid, a viral vector, or a nanoparticle. A “bacterial plasmid” is a small extrachromosomal DNA molecule that can be incorporated into another cell that is physically separated from the chromosomal DNA and is easily replicated. A “viral vector” is a vehicle originally from a virus used to artificially carry foreign genetic material into another cell or tissue.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of disease), during early onset (e.g., upon initial signs and symptoms of disease), or after an established development of the disease. Prophylactic administration can occur for several days to years prior to the manifestation of symptoms of an infection.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule. As used herein, a “trinucleotide repeat” refers to a repetitive sequence of three base-pair motifs in a DNA sequence. For example, the DNA sequence “GAAGAAGAAGAAGAA₍ₙ₎” contains a repetitive sequence of GAA nucleotides, wherein n=any number. The trinucleotide repeat can be located in a coding or non-coding region of a genome.

A “nucleic acid” is a chemical compound that serves as the primary information-carrying molecules in cells and make up the cellular genetic material. Nucleic acids are nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

A “guide RNA” refers to an RNA construct that functions as a guide for RNA or DNA targeting enzymes, such as an endonuclease, with which the guide RNA forms complexes. Guide RNAs can occur naturally but can also be constructed for targeted gene editing methodologies, such as CRISPR technologies.

The terms “percent identity” and “% identity,” as applied to nucleotide sequences, refer to the percentage of residue matches between at least two nucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known nucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined nucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

An endonuclease is an enzyme capable of cleaving the phosphodiester bonds between nucleotides of nucleic acids. Endonucleases can possess properties to cause double or single stranded breaks to target nucleic acids. Endonucleases are commonly used in gene editing methodologies, such as CRISPR technology, to modify a host genome.

As used herein, “downstream” refers to the relative position of a genetic sequence, either DNA or RNA. Downstream relates to the 5′ to 3′ direction relative to the start site of transcription, wherein downstream is usually closer to the 3′ end of a genetic sequence.

As used herein, “upstream” refers to the relative position of a genetic sequence, either DNA or RNA. Upstream relates to the 5′ to 3′ direction relative to the start site of transcription, wherein upstream is usually closer to the 5′ end of a genetic sequence.

An “intron” refers to a DNA sequence located between expressed gene regions, or exons, that are initially copied or transcribed into RNA, but is eventually cut out of the final, or mature, RNA sequence. Introns are usually not expressed and are not translated into amino acids. Introns function to allow for alternative splicing, or the ability of generating multiple forms of a protein from a single DNA or RNA sequence.

### Gene Editing Vectors

The present disclosure provides a gene editing vector targeting trinucleotide repeats within an intronic region of the fibroblast growth factor 14 (fgf14) gene.

In one aspect, disclosed herein is a gene editing vector comprising a nucleic acid sequence encoding an endonuclease, a first guide RNA (gRNA) comprising SEQ ID NO: 1, SEQ ID NO: 2, SEQ ID NO: 3, SEQ ID NO: 4, or SEQ ID NO: 5, a second gRNA comprising SEQ ID NO: 6, SEQ ID NO: 7, SEQ ID NO: 8, SEQ ID NO: 9, or SEQ ID NO: 10, wherein the first and second gRNAs are complementary to a first and second genomic loci in a nucleus of a cell that together flank a GAA repeat in intron 1 of a fibroblast growth factor 14 (FGF14) gene.

In some embodiments, the gene editing vector is delivered in a gene editing system, including but not limited to a CRISPR gene editing system. In some embodiments, the endonuclease is a Cas9 or Cpf1 endonuclease. In other embodiments, the endonuclease includes, but is not limited to Cas3, Cas5, Cas6, Cas7, Cas12a, Cas12d (CasY), Cas12e (CasX), Cas13, Cas 14, or variants thereof. In some embodiments, the nucleic acid comprises DNA.

In some embodiments, the nucleic acid comprises at least 50% identity of SEQ ID NO: 11. In other embodiments, the nucleic acid comprises a sequence of at least 50%, 55%, 60%, 65%, 70%, 75%, 80%, 85%, 90%, 95%, 99%, or 100% identical to SEQ ID NO: 11.

In some embodiments, the nucleic acid comprises at least 50% identity of SEQ ID NO: 12. In other embodiments, the nucleic acid comprises a sequence of at least 50%, 55%, 60%, 65%, 70%, 75%, 80%, 85%, 90%, 95%, 99%, or 100% identical to SEQ ID NO: 12.

In some embodiments, the nucleic acid comprises at least 50% identity of SEQ ID NO: 13. In other embodiments, the nucleic acid comprises a sequence of at least 50%, 55%, 60%, 65%, 70%, 75%, 80%, 85%, 90%, 95%, 99%, or 100% identical to SEQ ID NO: 13.

In some embodiments, the nucleic acid comprises at least 50% identity of SEQ ID NO: 14. In other embodiments, the nucleic acid comprises a sequence of at least 50%, 55%, 60%, 65%, 70%, 75%, 80%, 85%, 90%, 95%, 99%, or 100% identical to SEQ ID NO: 14.

In some embodiments, the nucleic acid comprises at least 50% identity of SEQ ID NO: 15. In other embodiments, the nucleic acid comprises a sequence of at least 50%, 55%, 60%, 65%, 70%, 75%, 80%, 85%, 90%, 95%, 99%, or 100% identical to SEQ ID NO: 15.

In some embodiments, the nucleic acid comprises at least 50% identity of SEQ ID NO: 16. In other embodiments, the nucleic acid comprises a sequence of at least 50%, 55%, 60%, 65%, 70%, 75%, 80%, 85%, 90%, 95%, 99%, or 100% identical to SEQ ID NO: 16.

In some embodiments, the nucleic acid comprises at least 50% identity of SEQ ID NO: 17. In other embodiments, the nucleic acid comprises a sequence of at least 50%, 55%, 60%, 65%, 70%, 75%, 80%, 85%, 90%, 95%, 99%, or 100% identical to SEQ ID NO: 17.

In some embodiments, the nucleic acid comprises at least 50% identity of SEQ ID NO: 18. In other embodiments the nucleic acid comprises a sequence of at least 50%, 55%, 60%, 65%, 70%, 75%, 80%, 85%, 90%, 95%, 99%, or 100% identical to SEQ ID NO: 18.

In some embodiments, the nucleic acid comprises at least 50% identity of SEQ ID NO: 19. In other embodiments, the nucleic acid comprises a sequence of at least 50%, 55%, 60%, 65%, 70%, 75%, 80%, 85%, 90%, 95%, 99%, or 100% identical to SEQ ID NO: 19.

In some embodiments, the nucleic acid comprises at least 50% identity of SEQ ID NO: 20. In other embodiments, the nucleic acid comprises a sequence of at least 50%, 55%, 60%, 65%, 70%, 75%, 80%, 85%, 90%, 95%, 99%, or 100% identical to SEQ ID NO: 20.

In one embodiment, the nucleic acid encoding the first gRNA comprises SEQ ID NO: 11, SEQ ID NO: 12, SEQ ID NO: 13, SEQ ID NO: 14, or SEQ ID NO: 15, or a fragment thereof. In one embodiment, the nucleic acid encoding the second gRNA comprises SEQ ID NO: 16, SEQ ID NO: 17, SEQ ID NO: 18, SEQ ID NO: 19, or SEQ ID NO: 20, or a fragment thereof.

In some embodiments, the gene editing vector is a viral vector. In some embodiments, the gene editing vector is an adeno-associated virus (AAV) vector. In some embodiments, the gene editing vector is an adenovirus (AdV or AV) vector. In some embodiments, the gene editing vector is a lentivirus vector. In some embodiments, the gene editing vector is a retrovirus vector. In some embodiments, the gene editing vector is a herpes simplex virus (HSV) vector.

In some embodiments, the gene editing vector is a non-viral vector. In some embodiments, the gene editing vector is a DNA plasmid. In some embodiments, the gene editing vector is a nanoparticle. In some embodiments, the nanoparticle is a polymer nanoparticle. In some embodiments, the nanoparticle is a lipid nanoparticle (LNP).

In one embodiment, one gene editing vector comprises the nucleic acid encoding the endonuclease, the first gRNA, the second gRNA, or combinations thereof. In some embodiments, more than one gene editing vector comprises the nucleic acid encoding the endonuclease, the first gRNA, the second gRNA, or combinations thereof. In some embodiments, more than one gene editing vector is delivered together to the cell. In some embodiments, more than one gene editing vector is delivered separately to the cell.

In one embodiment, the gene editing vector comprises a paired combination of SEQ ID NO: 11, SEQ ID NO: 12, SEQ ID NO: 13, SEQ ID NO: 14, or SEQ ID NO: 15 paired with SEQ ID NO: 16, SEQ ID NO: 17, SEQ ID NO: 18, SEQ ID NO: 19, or SEQ ID NO: 20.

In some embodiments, SEQ ID NO: 11 is paired with SEQ ID NO: 16. In some embodiments, SEQ ID NO: 11 is paired with SEQ ID NO: 17. In some embodiments, SEQ ID NO: 11 is paired with SEQ ID NO: 18. In some embodiments, SEQ ID NO: 11 is paired with SEQ ID NO: 19. In some embodiments, SEQ ID NO: 11 is paired with SEQ ID NO: 20.

In some embodiments, SEQ ID NO: 12 is paired with SEQ ID NO: 16. In some embodiments, SEQ ID NO: 12 is paired with SEQ ID NO: 17. In some embodiments, SEQ ID NO: 12 is paired with SEQ ID NO: 18. In some embodiments, SEQ ID NO: 12 is paired with SEQ ID NO: 19. In some embodiments, SEQ ID NO: 12 is paired with SEQ ID NO: 20.

In some embodiments, SEQ ID NO: 13 is paired with SEQ ID NO: 16. In some embodiments, SEQ ID NO: 13 is paired with SEQ ID NO: 17. In some embodiments, SEQ ID NO: 13 is paired with SEQ ID NO: 18. In some embodiments, SEQ ID NO: 13 is paired with SEQ ID NO: 19. In some embodiments, SEQ ID NO: 13 is paired with SEQ ID NO: 20.

In some embodiments, SEQ ID NO: 14 is paired with SEQ ID NO: 16. In some embodiments, SEQ ID NO: 14 is paired with SEQ ID NO: 17. In some embodiments, SEQ ID NO: 14 is paired with SEQ ID NO: 18. In some embodiments, SEQ ID NO: 14 is paired with SEQ ID NO: 19. In some embodiments, SEQ ID NO: 14 is paired with SEQ ID NO: 20.

In some embodiments, SEQ ID NO: 15 is paired with SEQ ID NO: 16. In some embodiments, SEQ ID NO: 15 is paired with SEQ ID NO: 17. In some embodiments, SEQ ID NO: 15 is paired with SEQ ID NO: 18. In some embodiments, SEQ ID NO: 15 is paired with SEQ ID NO: 19. In some embodiments, SEQ ID NO: 15 is paired with SEQ ID NO: 20.

In some embodiments, the gene editing vector removes the entire GAA repeat locus including at least: chr13: 102,813,925-102,814,074 (GRCh37).

In some embodiments, the gene editing system is delivered on one viral vector. In some embodiments, the gene editing system is delivered on more than one viral vector. In some embodiments, the gene editing system is delivered on two viral vectors. In some embodiments, the gene editing system is delivered on three viral vectors.

In some embodiments, the cell originates from the central nervous system. In some embodiments, the cell originates from the cerebellum.

### Gene Editing Systems

The present disclosure provides a gene editing system targeting trinucleotide repeats within an intronic region of the fibroblast growth factor 14 gene.

Genome editing or gene editing refers to a group of technologies that allows one in the art the ability to change a host's DNA sequence. These technologies allow genetic materials to be added, removed, or altered at specific genome locations, or genomic loci. These approaches are highly versatile practices allowing for diagnosing, treating, and/or prevention of genetic diseases or disorders.

In one aspect, disclosed herein is a gene editing system comprising a first guide RNA (gRNA) or a first nucleic acid sequence encoding the first gRNA, a second gRNA or a second nucleic acid sequence encoding the second gRNA, and an endonuclease or a third nucleic acid sequence encoding the endonuclease; wherein the first and second gRNAs target the endonuclease to a first and a second genomic loci in a nucleus of a cell that together flank a GAA repeat in intron 1 of a fibroblast growth factor 14 (FGF14) gene.

In some aspects, disclosed herein is a gene editing system comprising a ribonucleoprotein (RNP) complex comprising an endonuclease and two guide RNAs, wherein the two gRNAs comprise a first guide RNA (gRNA) or a first nucleic acid sequence encoding the first gRNA; and a second gRNA or a second nucleic acid sequence encoding the second gRNA; wherein the first and second gRNAs target the endonuclease to a first and a second genomic loci in a nucleus of a cell that together flank a GAA repeat in intron 1 of a fibroblast growth factor 14 (FGF14) gene.

“CRISPR” (Clustered Regularly Interspaced Short Palindromic Repeats) loci refers to certain genetic loci encoding components of DNA cleavage systems, for example, used by bacterial and archaeal cells to destroy foreign DNA (See for example, Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

In some embodiments, the gene editing system is a CRISPR gene editing system. In some embodiments, the gene editing system comprises a Cas9 or Cpf1 endonuclease. In some embodiments, the gene editing system includes, but is not limited to Cas3, Cas5, Cas6, Cas7, Cas12a, Cas12d (CasY), Cas12e (CasX), Cas13, Cas 14, or variants thereof.

In some embodiments, the gene editing system comprises a Cas-gRNA complex, known as a ribonucleoprotein (RNP). In some embodiments, the RNP complex contains one or more gRNAs comprising SEQ ID NO: 1, SEQ ID NO: 2, SEQ ID NO: 3, SEQ ID NO: 4, or SEQ ID NO: 5 upstream (5′) of the target genomic locus. In some embodiments, the RNP complex contains one or more gRNAs comprising SEQ ID NO: 6, SEQ ID NO: 7, SEQ ID NO: 8, SEQ ID NO: 9, or SEQ ID NO: downstream (3′) of the target genomic locus.

In some embodiments, the first gRNA comprises at least 50% identity of SEQ ID NO: 1. In other embodiments, the first gRNA comprises a sequence of at least 50%, 55%, 60%, 65%, 70%, 75%, 80%, 85%, 90%, 95%, 99%, or 100% identical to SEQ ID NO: 1.

In some embodiments, the first gRNA comprises at least 50% identity of SEQ ID NO: 2. In other embodiments, the first gRNA comprises a sequence of at least 50%, 55%, 60%, 65%, 70%, 75%, 80%, 85%, 90%, 95%, 99%, or 100% identical to SEQ ID NO: 2.

In some embodiments, the first gRNA comprises at least 50% identity of SEQ ID NO: 3. In other embodiments, the first gRNA comprises a sequence of at least 50%, 55%, 60%, 65%, 70%, 75%, 80%, 85%, 90%, 95%, 99%, or 100% identical to SEQ ID NO: 3.

In some embodiments, the first gRNA comprises at least 50% identity of SEQ ID NO: 4. In other embodiments, the first gRNA comprises a sequence of at least 50%, 55%, 60%, 65%, 70%, 75%, 80%, 85%, 90%, 95%, 99%, or 100% identical to SEQ ID NO: 4.

In some embodiments, the first gRNA comprises at least 50% identity of SEQ ID NO: 5. In other embodiments, the first gRNA comprises a sequence of at least 50%, 55%, 60%, 65%, 70%, 75%, 80%, 85%, 90%, 95%, 99%, or 100% identical to SEQ ID NO: 5.

In some embodiments, the second gRNA comprises at least 50% identity of SEQ ID NO: 6. In other embodiments, the second gRNA comprises a sequence of at least 50%, 55%, 60%, 65%, 70%, 75%, 80%, 85%, 90%, 95%, 99%, or 100% identical to SEQ ID NO: 6.

In some embodiments, the second gRNA comprises at least 50% identity of SEQ ID NO: 7. In other embodiments, the second gRNA comprises a sequence of at least 50%, 55%, 60%, 65%, 70%, 75%, 80%, 85%, 90%, 95%, 99%, or 100% identical to SEQ ID NO: 7.

In some embodiments, the second gRNA comprises at least 50% identity of SEQ ID NO: 8.

In other embodiments, the second gRNA comprises a sequence of at least 50%, 55%, 60%, 65%, 70%, 75%, 80%, 85%, 90%, 95%, 99%, or 100% identical to SEQ ID NO: 8.

In some embodiments, the second gRNA comprises at least 50% identity of SEQ ID NO: 9. In other embodiments, the second gRNA comprises a sequence of at least 50%, 55%, 60%, 65%, 70%, 75%, 80%, 85%, 90%, 95%, 99%, or 100% identical to SEQ ID NO: 9.

In some embodiments, the second gRNA comprises at least at least 50% identity of SEQ ID NO: 10. In other embodiments, the second gRNA comprises a sequence of at least 50%, 55%, 60%, 65%, 70%, 75%, 80%, 85%, 90%, 95%, 99%, or 100% identical to SEQ ID NO: 10.

In some embodiments, the first gRNA comprises SEQ ID NO: 1, SEQ ID NO: 2, SEQ ID NO: 3, SEQ ID NO: 4, or SEQ ID NO: 5. In other embodiments, the second gRNA comprises SEQ ID NO: 6, SEQ ID NO: 7, SEQ ID NO: 8, SEQ ID NO: 9, or SEQ ID NO: 10.

In some embodiments, the gene editing system comprises a pair of two single gRNAs comprising SEQ ID NO: 1, SEQ ID NO: 2, SEQ ID NO: 3, SEQ ID NO: 4, or SEQ ID NO: 5 paired with SEQ ID NO: 6, SEQ ID NO: 7, SEQ ID NO: 8, SEQ ID NO: 9, or SEQ ID NO: 10.

In some embodiments, SEQ ID NO: 1 is paired with SEQ ID NO: 6. In some embodiments, SEQ ID NO: 1 is paired with SEQ ID NO: 7. In some embodiments, SEQ ID NO: 1 is paired with SEQ ID NO: 8. In some embodiments, SEQ ID NO: 1 is paired with SEQ ID NO: 9. In some embodiments, SEQ ID NO: 1 is paired with SEQ ID NO: 10.

In some embodiments, SEQ ID NO: 2 is paired with SEQ ID NO: 6. In some embodiments, SEQ ID NO: 2 is paired with SEQ ID NO: 7. In some embodiments, SEQ ID NO: 2 is paired with SEQ ID NO: 8. In some embodiments, SEQ ID NO: 2 is paired with SEQ ID NO: 9. In some embodiments, SEQ ID NO: 2 is paired with SEQ ID NO: 10.

In some embodiments, SEQ ID NO: 3 is paired with SEQ ID NO: 6. In some embodiments, SEQ ID NO: 3 is paired with SEQ ID NO: 7. In some embodiments, SEQ ID NO: 3 is paired with SEQ ID NO: 8. In some embodiments, SEQ ID NO: 3 is paired with SEQ ID NO: 9. In some embodiments, SEQ ID NO: 3 is paired with SEQ ID NO: 10.

In some embodiments, SEQ ID NO: 4 is paired with SEQ ID NO: 6. In some embodiments, SEQ ID NO: 4 is paired with SEQ ID NO: 7. In some embodiments, SEQ ID NO: 4 is paired with SEQ ID NO: 8. In some embodiments, SEQ ID NO: 4 is paired with SEQ ID NO: 9. In some embodiments, SEQ ID NO: 4 is paired with SEQ ID NO: 10.

In some embodiments, SEQ ID NO: 5 is paired with SEQ ID NO: 6. In some embodiments, SEQ ID NO: 5 is paired with SEQ ID NO: 7. In some embodiments, SEQ ID NO: 5 is paired with SEQ ID NO: 8. In some embodiments, SEQ ID NO: 5 is paired with SEQ ID NO: 9. In some embodiments, SEQ ID NO: 5 is paired with SEQ ID NO: 10.

In one embodiment, the first gRNA comprising SEQ ID NO: 1, SEQ ID NO: 2, SEQ ID NO: 3, SEQ ID NO: 4, or SEQ ID NO: 5 targets upstream (5′) of a target genomic locus. In one embodiment, the second gRNA comprising SEQ ID NO: 6, SEQ ID NO: 7, SEQ ID NO: 8, SEQ ID NO: 9, or SEQ ID NO: 10 targets downstream (3′) of the target genomic locus.

In some embodiments, the first and second gRNAs are delivered together. In some embodiments, the first and second gRNAs are delivered separately. In some embodiments, the first and second gRNAs are delivered with the endonuclease. In some embodiments, the first and second gRNAs are delivered separately from the endonuclease.

In some embodiments, the first and second genomic loci are upstream (5′) of the GAA repeat and downstream (3′) of the GAA repeat, respectively.

It should be understood through the present disclosure that “GAA repeat”, “GAA trinucleotide”, “GAA trinucleotide repeat”, “GAA repeat expansion”, and “GAA expansion” can be used interchangeably, and are further understood to describe a repetitive DNA sequence comprising GAA (n), wherein n=any number greater than one. The trinucleotide repeat, GAA, can be removed from the fibroblast growth factor 14 (FGF14) gene. In some embodiments, the gene editing system of the present disclosure removes at least 100 GAA repeats. In other embodiments, the gene editing system removes 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more GAA repeats. In some embodiments, the gene editing system removes at least 250 GAA repeats. In other embodiments, the gene editing system removes at least 300 GAA repeats. In other embodiments, the gene editing system removes all the GAA repeats.

In some embodiments, the gene editing system comprises one or more nucleic acid constructs expressed in a vector. The vector(s) can also include any suitable number of regulatory/control elements, e.g., promoters, enhancers, introns, polyadenylation signals, Kozak consensus sequences, or internal ribosome entry sites (IRES). It should be understood that the nucleic acids encoding the endonuclease, the first gRNA, or the second gRNA, alone or in combination, can be placed within a viral or non-viral vector. In some embodiments, the gene editing system comprises one or more nucleic acid constructs expressed in an adeno-associated virus (AAV), an adenovirus, a lentivirus, and a retrovirus vector. In some embodiments, the gene editing system comprises one or more nucleic acid constructs expressed in DNA plasmid. In some embodiments, the gene editing system is delivered using a polymer, or a biodegradable polymer. In other embodiments, the gene editing system is delivered using polymers and polymeric matrices, non-polymeric matrices, or inorganic and organic excipients and diluents such as, but not limited to, calcium carbonate and sugar (for example, trehalose).

In some embodiments, the gene editing system is administered with a pharmaceutically acceptable carrier, salt, solvent, or excipient. In other embodiments, the gene editing system is administered with a nanoparticle. In some embodiments, the nanoparticle is a polymer nanoparticle. In some embodiments, the nanoparticle is a lipid nanoparticle (LPN).

In some embodiments, the endonuclease is an RNA-guided DNA endonuclease.

In some embodiments, the gene editing system removes the entire GAA repeat locus including at least: chr13: 102,813,925-102,814,074 (GRCh37).

### Methods of Gene Editing

The present disclosure provides methods of gene editing an FGF14 gene in a cell or subject.

In one aspect, disclosed herein is a method of gene editing of a fibroblast growth factor 14 (FGF14) gene in a cell or a subject, the method comprising targeting a first guide RNA (gRNA) and an endonuclease to a first genomic loci of the FGF14 gene, targeting a second gRNA and the endonuclease to a second genomic loci of the FGF14 gene, and removing a GAA repeat in intron 1 of the FGF14 gene.

In some embodiments, the method comprises a CRISPR gene editing system. In some embodiments, the method comprises a Cas9 or Cpf1 endonuclease. In some embodiments, the method includes, but is not limited to Cas3, Cas5, Cas6, Cas7, Cas12a, Cas12d (CasY), Cas12e (CasX), Cas13, Cas 14, or variants thereof.

In some embodiments, the first gRNA comprises at least 50% identity of SEQ ID NO: 1. In other embodiments, the first gRNA comprises a sequence of at least 50%, 55%, 60%, 65%, 70%, 75%, 80%, 85%, 90%, 95%, 99%, or 100% identical to SEQ ID NO: 1.

In some embodiments, the first gRNA comprises at least 50% identity of SEQ ID NO: 2. In other embodiments, the first gRNA comprises a sequence of at least 50%, 55%, 60%, 65%, 70%, 75%, 80%, 85%, 90%, 95%, 99%, or 100% identical to SEQ ID NO: 2.

In some embodiments, the first gRNA comprises at least 50% identity of SEQ ID NO: 3. In other embodiments, the first gRNA comprises a sequence of at least 50%, 55%, 60%, 65%, 70%, 75%, 80%, 85%, 90%, 95%, 99%, or 100% identical to SEQ ID NO: 3.

In some embodiments, the first gRNA comprises at least 50% identity of SEQ ID NO: 4. In other embodiments, the first gRNA comprises a sequence of at least 50%, 55%, 60%, 65%, 70%, 75%, 80%, 85%, 90%, 95%, 99%, or 100% identical to SEQ ID NO: 4.

In some embodiments, the first gRNA comprises at least 50% identity of SEQ ID NO: 5. In other embodiments, the first gRNA comprises a sequence of at least 50%, 55%, 60%, 65%, 70%, 75%, 80%, 85%, 90%, 95%, 99%, or 100% identical to SEQ ID NO: 5.

In some embodiments, the second gRNA comprises at least 50% identity of SEQ ID NO: 6. In other embodiments, the second gRNA comprises a sequence of at least 50%, 55%, 60%, 65%, 70%, 75%, 80%, 85%, 90%, 95%, 99%, or 100% identical to SEQ ID NO: 6.

In some embodiments, the second gRNA comprises at least 50% identity of SEQ ID NO: 7. In other embodiments, the second gRNA comprises a sequence of at least 50%, 55%, 60%, 65%, 70%, 75%, 80%, 85%, 90%, 95%, 99%, or 100% identical to SEQ ID NO: 7.

In some embodiments, the second gRNA comprises at least 50% identity of SEQ ID NO: 8. In other embodiments, the second gRNA comprises a sequence of at least 50%, 55%, 60%, 65%, 70%, 75%, 80%, 85%, 90%, 95%, 99%, or 100% identical to SEQ ID NO: 8.

In some embodiments, the second gRNA comprises at least 50% identity of SEQ ID NO: 9. In other embodiments, the second gRNA comprises a sequence of at least 50%, 55%, 60%, 65%, 70%, 75%, 80%, 85%, 90%, 95%, 99%, or 100% identical to SEQ ID NO: 9.

In some embodiments, the second gRNA comprises at least 50% identity of SEQ ID NO: 10. In other embodiments, the second gRNA comprises a sequence of at least 50%, 55%, 60%, 65%, 70%, 75%, 80%, 85%, 90%, 95%, 99%, or 100% identical to SEQ ID NO: 10.

In some embodiments, the first gRNA comprises SEQ ID NO: 1, SEQ ID NO: 2, SEQ ID NO: 3, SEQ ID NO: 4, or SEQ ID NO: 5. In other embodiments, the second gRNA comprises SEQ ID NO: 6, SEQ ID NO: 7, SEQ ID NO: 8, SEQ ID NO: 9, or SEQ ID NO: 10.

In some embodiments, the first and second gRNAs are delivered together. In some embodiments, the first and second gRNAs are delivered separately. In some embodiments, the first and second gRNAs are delivered with the endonuclease. In some embodiments, the first and second gRNAs are delivered separately from the endonuclease.

In some embodiments, the method removes at least 100 GAA repeats. In other embodiments, the method removes 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more GAA repeats. In some embodiments, the method removes at least 250 GAA repeats. In other embodiments, the method removes at least 300 GAA repeats.

In some embodiments, the method removes all GAA repeats. In some embodiments, the method removes a number of GAA repeats of any preceding embodiment and leaves less than 250 GAA repeats remaining at the target genomic locus. In some embodiments, the method leaves 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, or 249 GAA repeats remaining at the target genomic locus.

In some embodiments, the method comprises a Cas-gRNA complex, known as a ribonucleoprotein (RNP). In some embodiments, the RNP complex contains one or more gRNAs comprising SEQ ID NO: 1, SEQ ID NO: 2, SEQ ID NO: 3, SEQ ID NO: 4, or SEQ ID NO: 5 upstream (5′) of the target genomic locus. In some embodiments, the RNP complex contains one or more gRNAs comprising SEQ ID NO: 6, SEQ ID NO: 7, SEQ ID NO: 8, SEQ ID NO: 9, or SEQ ID NO: 10 downstream (3′) of the target genomic locus.

In other embodiments, the first and second genomic loci are upstream (5′) of the GAA repeat and downstream (3′) of the GAA repeat.

A spinocerebellar or cerebellar ataxia refers to a group of genetic neurological disorders, largely affecting the cerebellum region of the brain, as well as the spinal cord, to impair coordination of physical movements. These particular genetic defects can be detected early in life (i.e.: newborns or older) or become detectable at later stages of like (i.e.: greater than 50 years old). Further, these particular genetic defects can worsen over time and cause specific issues with coordination, including, but not limited to eye coordination, hand and leg coordination, mobility, and speech, and quality of life. An episodic ataxia refers to a group of related ataxias that also affect the neurological system and cause issue with physical movements. This type of ataxia presents with recurrent episodes of poor or absent coordination and balance. During these episodes and other aforementioned forms of ataxia, a subject can experience dizziness (vertigo), nausea, vomiting, migraine headaches, blurred or double vision, slurred speech, ringing ears (tinnitus), seizures, muscle weakness, and paralysis. In some embodiments, the method treats or prevents a cerebellar, a spinocerebellar, or an episodic ataxia in the subject, or a phenotypically related disorder. In other embodiments, the subject is a human.

Methods of Treating and/or Preventing Late-Onset Cerebellar Ataxia (LOCA)

In another aspect, disclosed herein is a method of treating or preventing a late-onset cerebellar ataxia (LOCA), the method comprising administering to a subject in need thereof a gene editing system to at least one cell in the subject, wherein the gene editing system comprises a first guide RNA (gRNA) or a first nucleic acid sequence encoding the first gRNA a second gRNA or a second nucleic acid sequence encoding the second gRNA and an endonuclease or a third nucleic acid sequence encoding the endonuclease wherein the first and second gRNAs target the endonuclease to a first and a second genomic loci in a nucleus of the cell that together flank a GAA repeat in intron 1 of a fibroblast growth factor 14 (FGF14) gene. It should be noted that late onset refers to the development, detection, and/or diagnosis of a disease or disorder after the age of 30. In some embodiments, LOCA is developed, detected, or diagnosed in a subject that is at least 30 years old. In other embodiments, LOCA is developed, detected, or diagnosed in a subject that is 50 years old. In other embodiments, LOCA is developed, detected, or diagnosed in a subject that is 60 years old. In other embodiments, LOCA is developed, detected, or diagnosed in a subject that is 65 years old. In some embodiments, LOCA is developed, detected, or diagnosed in a subject that is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 years old or older.

In one embodiment, the method of treating and/or preventing LOCA comprises a CRISPR gene editing system. In one embodiment, the method of treating and/or preventing LOCA comprises a CRISPR/Cas gene editing system. In other embodiments, the of treating and/or preventing LOCA comprises a Cas9 or Cpf1 endonuclease. In other embodiments, the of treating and/or preventing LOCA includes, but is not limited to Cas3, Cas5, Cas6, Cas7, Cas12a, Cas12d (CasY), Cas12e (CasX), Cas13, Cas 14, or variants thereof.

In some embodiments, the first gRNA comprises at least 50% identity of SEQ ID NO: 1. In other embodiments, the first gRNA comprises a sequence of at least 50%, 55%, 60%, 65%, 70%, 75%, 80%, 85%, 90%, 95%, 99%, or 100% identical to SEQ ID NO: 1.

In some embodiments, the first gRNA comprises at least 50% identity of SEQ ID NO: 2. In other embodiments, the first gRNA comprises a sequence of at least 50%, 55%, 60%, 65%, 70%, 75%, 80%, 85%, 90%, 95%, 99%, or 100% identical to SEQ ID NO: 2.

In some embodiments, the first gRNA comprises at least 50% identity of SEQ ID NO: 3. In other embodiments, the first gRNA comprises a sequence of at least 50%, 55%, 60%, 65%, 70%, 75%, 80%, 85%, 90%, 95%, 99%, or 100% identical to SEQ ID NO: 3.

In some embodiments, the first gRNA comprises at least 50% identity of SEQ ID NO: 4. In other embodiments, the first gRNA comprises a sequence of at least 50%, 55%, 60%, 65%, 70%, 75%, 80%, 85%, 90%, 95%, 99%, or 100% identical to SEQ ID NO: 4.

In some embodiments, the first gRNA comprises at least 50% identity of SEQ ID NO: 5. In other embodiments, the first gRNA comprises a sequence of at least 50%, 55%, 60%, 65%, 70%, 75%, 80%, 85%, 90%, 95%, 99%, or 100% identical to SEQ ID NO: 5.

In some embodiments, the second gRNA comprises at least 50% identity of SEQ ID NO: 6. In other embodiments, the second gRNA comprises a sequence of at least 50%, 55%, 60%, 65%, 70%, 75%, 80%, 85%, 90%, 95%, 99%, or 100% identical to SEQ ID NO: 6.

In some embodiments, the second gRNA comprises at least 50% identity of SEQ ID NO: 7. In other embodiments, the second gRNA comprises a sequence of at least 50%, 55%, 60%, 65%, 70%, 75%, 80%, 85%, 90%, 95%, 99%, or 100% identical to SEQ ID NO: 7.

In some embodiments, the second gRNA comprises at least 50% identity of SEQ ID NO: 8. In other embodiments, the second gRNA comprises a sequence of at least 50%, 55%, 60%, 65%, 70%, 75%, 80%, 85%, 90%, 95%, 99%, or 100% identical to SEQ ID NO: 8.

In some embodiments, the second gRNA comprises at least 50% identity of SEQ ID NO: 9. In other embodiments, the second gRNA comprises a sequence of at least 50%, 55%, 60%, 65%, 70%, 75%, 80%, 85%, 90%, 95%, 99%, or 100% identical to SEQ ID NO: 9.

In some embodiments, the second gRNA comprises at least 50% identity of SEQ ID NO: 10. In other embodiments, the second gRNA comprises a sequence of at least 50%, 55%, 60%, 65%, 70%, 75%, 80%, 85%, 90%, 95%, 99%, or 100% identical to SEQ ID NO: 10.

In some embodiments, the first gRNA comprises SEQ ID NO: 1, SEQ ID NO: 2, SEQ ID NO: 3, SEQ ID NO: 4, or SEQ ID NO: 5. In other embodiments, the second gRNA comprises SEQ ID NO: 6, SEQ ID NO: 7, SEQ ID NO: 8, SEQ ID NO: 9, or SEQ ID NO: 10.

In some embodiments, the first and second gRNAs are delivered together. In some embodiments, the first and second gRNAs are delivered separately. In some embodiments, the first and second gRNAs are delivered with the endonuclease. In some embodiments, the first and second gRNAs are delivered separately from the endonuclease.

In some embodiments, the endonuclease partially or completely removes the GAA repeat at intron 1 of the FGF14 gene. In other embodiments, the FGF14 gene is reassembled by a recombination event. As used herein, a “recombination” event refers to the rejoining process of two or more DNA sequences that have been cut or separated by an endonuclease enzyme.

In some embodiments, the method of treating and/or preventing LOCA removes at least 100 GAA repeats. In other embodiments, the method of treating and/or preventing LOCA removes 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more GAA repeats. In some embodiments, the method of treating and/or preventing LOCA removes at least 250 GAA repeats. In other embodiments, the method of treating and/or preventing LOCA removes at least 300 GAA repeats.

In some embodiments, the method of treating and/or preventing LOCA removes all GAA repeats. In some embodiments, the method of treating and/or preventing LOCA removes a number of GAA repeats of any preceding embodiment and leaves less than 250 GAA repeats remaining at the target genomic locus. In some embodiments, the method of treating and/or preventing LOCA leaves 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, or 249 GAA repeats remaining at the target genomic locus.

In some embodiments, the first and second genomic loci are upstream (5′) of the GAA repeat and downstream (3′) of the GAA repeat.

In some embodiments, the method of treating and/or preventing LOCA comprises one or more nucleic acid constructs expressed in a vector. The vector(s) can also include any suitable number of regulatory/control elements, e.g., promoters, enhancers, introns, polyadenylation signals, Kozak consensus sequences, or internal ribosome entry sites (IRES). It should be understood that the nucleic acids encoding the endonuclease, the first gRNA, or the second gRNA, alone or in combination, can be placed within a viral or non-viral vector. In some embodiments, the method comprises the gene editing system comprising one or more nucleic acid constructs expressed in an adeno-associated virus (AAV), an adenovirus, a lentivirus, a retrovirus vector, or a herpes simplex virus. In some embodiments, the method comprises the gene editing system comprising one or more nucleic acid constructs expressed in DNA plasmid. In some embodiments, the method comprises the gene editing system delivered using a polymer, or a biodegradable polymer. In other embodiments, the gene editing system is delivered using polymers and polymeric matrices, non-polymeric matrices, or inorganic and organic excipients and diluents such as, but not limited to, calcium carbonate and sugar (for example, trehalose). In other embodiments, the gene editing system is administered with a pharmaceutically acceptable carrier, salt, solvent, or excipient.

In other embodiments, the subject is a human.

In some embodiments, the method treats or prevents cells in the central nervous system, including, but not limited to cells of the cerebellum.

### Administration

Genome editing systems and methods disclosed herein can be administered to subjects by any suitable mode or route, whether local or systemic. Systemic modes of administration include oral and parenteral routes. Parenteral routes include, by way of example, intravenous, intraarterial, intramuscular, intradermal, subcutaneous, intranasal, and intraperitoneal routes.

Skilled artisans will appreciate that different components of genome editing systems can be delivered together or separately and simultaneously or non-simultaneously. Separate and/or asynchronous delivery of genome editing system components may be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

The vector or system of any preceding aspect may be administered in such amounts, time, and route deemed necessary in order to achieve the desired result. The exact amount of the vector or system of any preceding aspect will vary from subject to subject, depending on the species, age, and general condition of the subject, the severity of the disease, the particular GAA trinucleotide repeat, its mode of administration, its mode of activity, and the like. The vector or system of any preceding aspect is preferably formulated in dosage unit form for ease of administration and uniformity of dosage. It will be understood, however, that the total daily usage of the vector or system of any preceding aspect will be decided by the attending physician within the scope of sound medical judgment. The specific therapeutically effective dose level for any particular subject will depend upon a variety of factors including the disease being treated and the severity of the GAA trinucleotide repeats; the activity of the vector or system of any preceding aspect employed; the specific vector or system of any preceding aspect employed; the age, body weight, general health, sex and diet of the patient; the time of administration, route of administration, and rate of excretion of the specific vector or system of any preceding aspect employed; the duration of the treatment; drugs used in combination or coincidental with the specific vector or system of any preceding aspect employed; and like factors well known in the medical arts.

The vector or system of any preceding aspect may be administered by a route deemed suitable to produce the desired effect of removing GAA trinucleotide repeats. In general, the most appropriate route of administration will depend upon a variety of factors including the nature of the vector or system of any preceding aspect (e.g., its stability in the subject's body), the condition of the subject (e.g., whether the subject is able to tolerate the selected route of administration), etc.

The exact amount of vector or system of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is vector or system of any preceding aspect and a pharmaceutically acceptable carrier selected from an excipient, a diluent, a salt, a buffer, a stabilizer, a lipid, an emulsion, a nanoparticle, and a cream. One or more active agents (e.g. the gene editing vector or system) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the vector or system of any preceding aspect is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the vector or system of any preceding aspect is administered daily. In some embodiments, the vector or system of any preceding aspect is administered every day, every 2 days, every 3 days, every 4 days, every 5 days, every 6 days, every 7 days, or more. In some embodiments, the vector or system of any preceding aspect is administered every week, every 2 weeks, every 3 weeks, every 4 weeks, or more. In some embodiments, the vector or system of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the vector or system of any preceding aspect is administered every year, every 2 years, every 3 years, every 4 years, every 5 years, or more.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Overview of Repeat Trinucleotide Expansion in First Intron of FGF14

The late-onset cerebellar ataxias (LOCAs) have largely resisted molecular diagnosis. Genomes were sequenced from six persons with unsolved autosomal dominant LOCA who were members of three French Canadian families and identified a candidate pathogenic repeat expansion. The association between the repeat expansion and disease in 66 French Canadian index patients and 209 controls and in 228 German index patients and 199 controls was tested. A search was conducted for the repeat expansion in 20 Australian and 31 Indian index patients. The gene and protein expression was tested in two postmortem cerebellum specimens and two induced pluripotent stem cell (iPSC)—derived motor neuron cell lines. In the six French Canadian patients, a GAA repeat expansion was identified in the first intron of FGF14, which encodes fibroblast growth factor 14. Cosegregation of the repeat expansion with disease in the families supported a pathogenic threshold of at least 250 GAA repeats ([GAA]_{≥}250). There was significant association between FGF14 (GAA)₂₅₀ expansions and LOCA in the French Canadian series (odds ratio, 105.60; 95% confidence interval [CI], 31.09 to 334.20; P<0.001) and in the German series (odds ratio, 8.76; 95% CI, 3.45 to 20.84; P<0.001). The repeat expansion was present in 61%, 18%, 15%, and 10% of French Canadian, German, Australian, and Indian index patients, respectively. In total, 128 patients with LOCA who carried an FGF14 (GAA)₂₅₀ expansion were identified. Postmortem cerebellum specimens and iPSC-derived motor neurons from patients showed reduced expression of FGF14 RNA and protein. In conclusion, a dominantly inherited deep intronic GAA repeat expansion in FGF14 was found to be associated with LOCA.

### Example 2: Deep Intronic FGF14 GAA Repeat Expansion in Late-Onset Cerebellar Ataxia

Late-onset cerebellar ataxias (LOCAs) are a heterogeneous group of neurodegenerative disorders manifesting as a progressive cerebellar syndrome that develops after 30 years of age. The prevalence of LOCA is approximately 1 to 3 per 100,000 population, and molecular testing yields negative results in almost 75% of patients with LOCA. This is explained in part by the limitations of standard next-generation sequencing analysis for the identification of certain sequence variations, such as tandem repeat expansions. Genome sequencing and bioinformatics tools were used to search for novel pathogenic repeat expansions in patients with LOCA.

### Methods

Enrollment, Imaging, and Neuropathological Assessment. To be eligible for inclusion in the study, patients needed to have progressive ataxia with onset at or after the age of 30 years; no clinical features suggestive of multiple system atrophy, cerebellar subtype (MSA-c); exclusion of acquired disease; and, as appropriate, negative results on testing with an ataxia gene panel and on screening for repeat expansions that cause common spinocerebellar ataxias, fragile X-associated tremor/ataxia syndrome, Friedreich ataxia, and CANVAS (cerebellar ataxia, neuropathy, and vestibular areflexia syndrome).

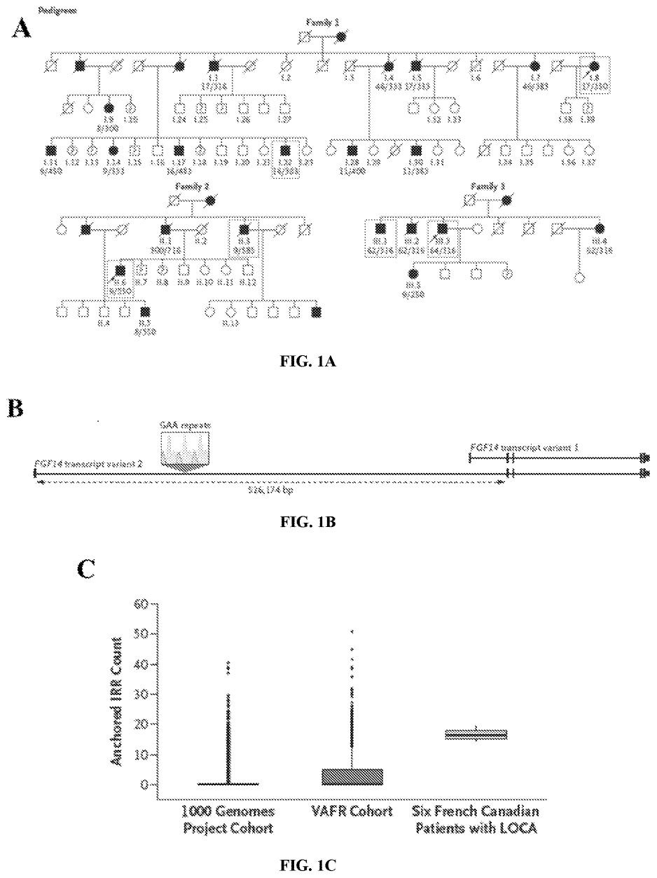

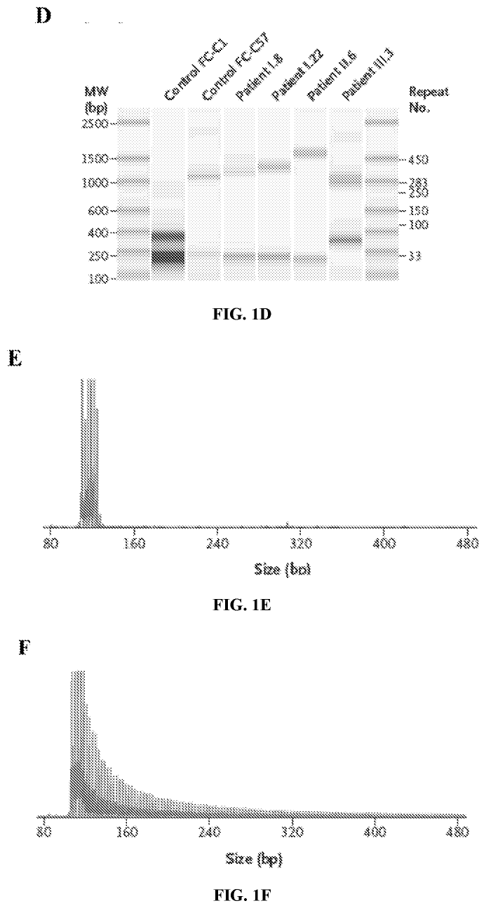

Two persons with autosomal dominant LOCA from each of three large French Canadian families (Families I, II, and III; FIG. 1A) made up the discovery cohort. Four independent validation cohorts of 66 French Canadian, 228 German, 20 Australian and 31 Indian index patients were recruited. The control cohorts consisted of 209 French Canadian and 199 German persons who were neurologically healthy or who were reported not to have ataxia.

Aside from 14 patients whose ancestry was determined by analysis of next-generation sequencing data, participants reported their own race and ethnic group. Information on the representativeness of the study participants is presented in Table 4, available with the full text of this article at NEJM.org. Brain images were obtained by magnetic resonance imaging (MRI), and comprehensive neuropathological examinations were conducted on the brains from two deceased French Canadian patients. Written informed consent was obtained from all the participants, and approval was obtained from all local institutional review boards.

Genetic Studies. Genomes of the six French Canadian patients from the discovery cohort were sequenced. A subsequent search for pathogenic repeat expansions was conducted with the use of Expansion Hunter Denovo software, version 0.6.2. Participants in the 1000 Genome Project and the Vanderbilt Atrial Fibrillation Registry served as controls. The FGF14 repeat locus was amplified by long-range polymerase chain reaction (PCR). The number of repeat units was estimated by means of agarose gel electrophoresis. The motif of the repeat locus in patients and controls who had large amplification products on long-range PCR was analyzed by targeted long read nanopore sequencing. For patients with insufficient DNA for nanopore sequencing, repeat-primed PCR were used to ascertain the presence of a GAA repeat expansion. Targeted long read nanopore sequencing was performed on specimens from 104 patients (55 French Canadian, 44 German, and 5 Australian) and 18 controls.

Association Testing. The association between LOCA and FGF14 GAA expansions of 250 or more repeats ([GAA]≥250) was tested, as measured by agarose gel electrophoresis of PCR-amplification products, in two independent case-control series consisting of 66 French Canadian cases and 209 control and 228 German cases and 199 controls. Greater than 250 repeat units was used for association studies following the segregation study within the three large French Canadian families (discovery cohort), in which the smallest expansion among symptomatic persons was (GAA)₂₅₀.

Functional analyses in postmortem cerebellar tissue and induced pluripotent stem cell-derived motor neurons. Postmortem cerebellar tissue was obtained from two patients of Spanish origin carrying an FGF14 GAA repeat expansion uncovered after screening of postmortem brain specimens from 15 patients of European descent with idiopathic LOCA and 7 controls of European descent without ataxia. The brain specimens were obtained through the Queen Square Brain Bank for Neurological Disorders, London, and the Neurological Tissue Brain Bank of the Hospital Clínic-IDIBAPS (Instituto de Investigaciones Biomédicas August Pi i Sunyer) Biobanc, Barcelona (Table 5).

Induced pluripotent stem cell (iPSC) lines-one from a patient carrying a (GAA)₃₈₃ expansion, one from a patient homozygous for (GAA)₃₀₀ expansions, and two from unaffected persons of European descent-generated and differentiated into motor neurons using an established protocol. While neither of the patients had clinical features consistent with motor neuron disease, the challenges of establishing mature Purkinje cell lines in culture⁹ led to the generation of iPSC-derived motor neurons.

Statistical Analysis. A Fisher's exact test was used for tests of association. No method to adjust for multiplicity of inferences was prespecified. The widths of the confidence intervals have not been adjusted for multiplicity, and therefore the confidence intervals should not be used to reject or not reject effects.

### Results

Identification of a GAA Repeat Expansion in FGF14. Six patients with unsolved autosomal dominant LOCA were selected from the three large French Canadian families (FIG. 1A) for genome sequencing because they shared a similar phenotype that included episodic features and downbeat nystagmus. Linkage analysis in Family I had not identified candidate loci as a result of the likely presymptomatic status of some of the younger members. Initial analysis of the genome sequences revealed no rare pathogenic variants segregating across all three pedigrees.

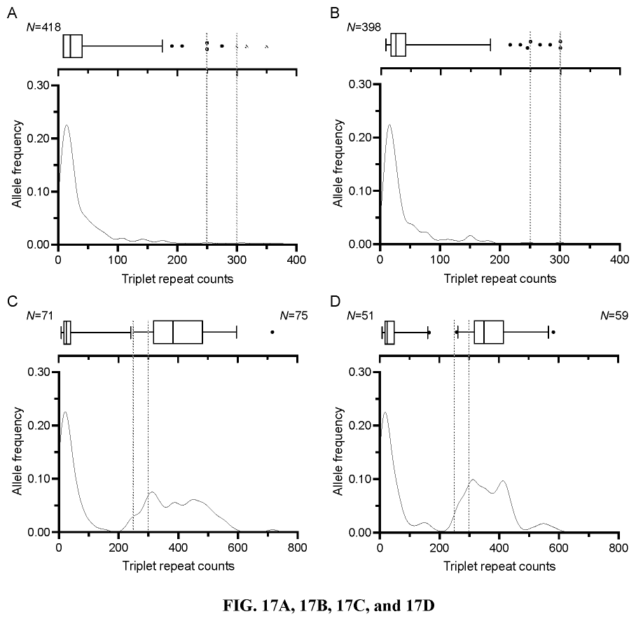

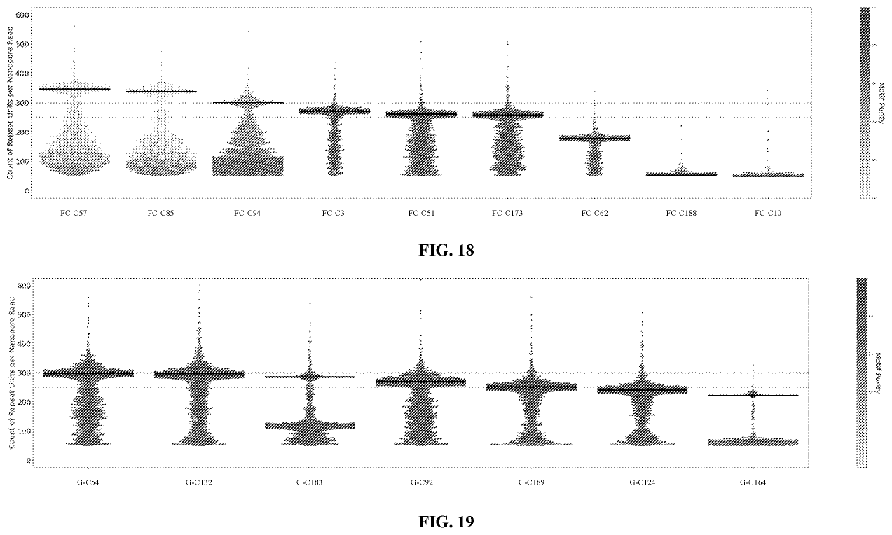

Next, ExpansionHunter Denovo was used and identified a putative heterozygous GAA repeat expansion shared among all six patients at position chr13: 102,813,925-102,814,074 (GRCh37) (FIGS. 1B, 1C, and 16). This locus is located in intron 1 of FGF14, which is included in pre-mRNA transcript 2 and not in pre-mRNA transcript 1 (FIG. 1B). Exonic point and frameshift variants in FGF14 have previously been shown to cause autosomal dominant spinocerebellar ataxia 27 (SCA27). Long-range PCR and repeat-primed PCR showed a large heterozygous GAA repeat expansion in each of the six patients and in 15 affected relatives (FIGS. 1D, 1E, 1F, and 17). Sanger sequencing confirmed the repeat motif to be GAA. The smallest expansion in any affected family member was (GAA)₂₅₀.

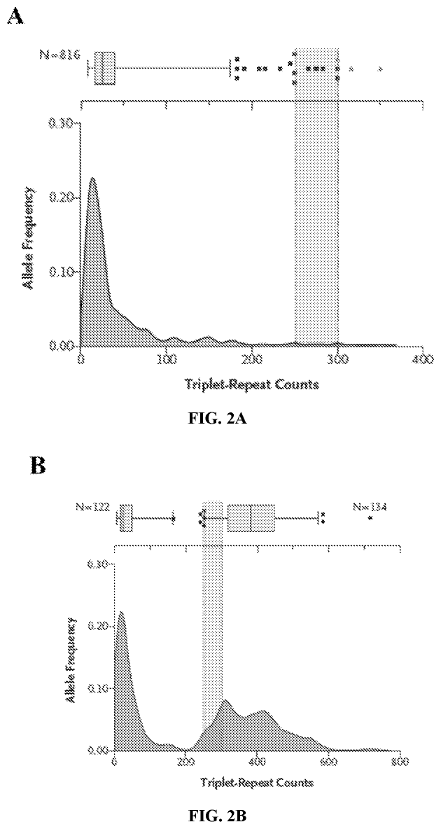

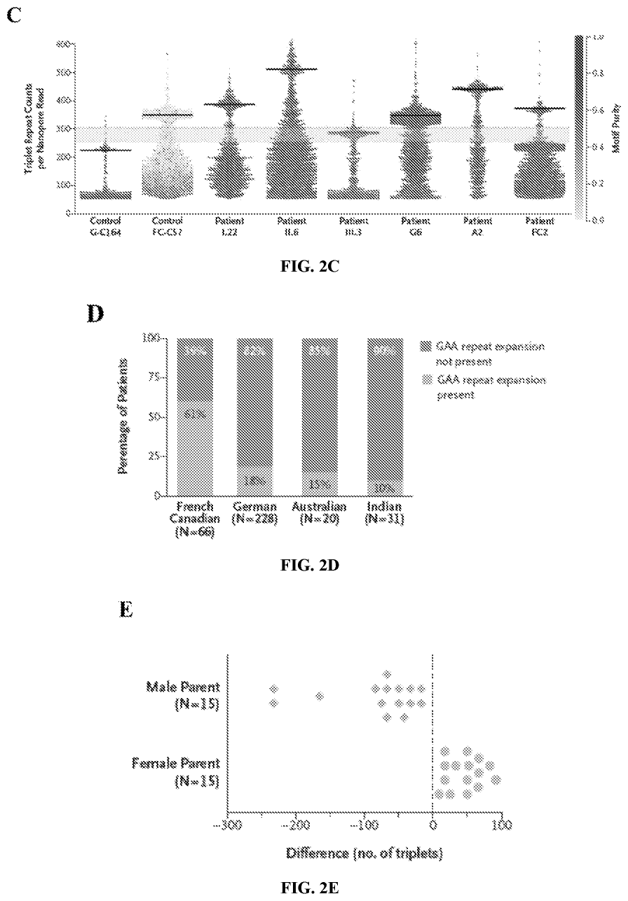

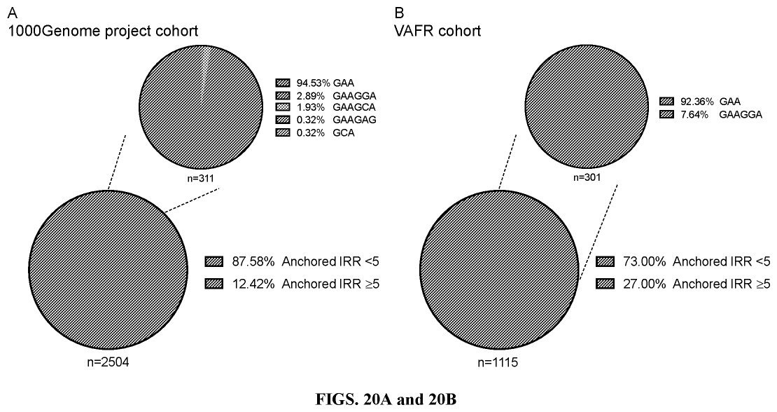

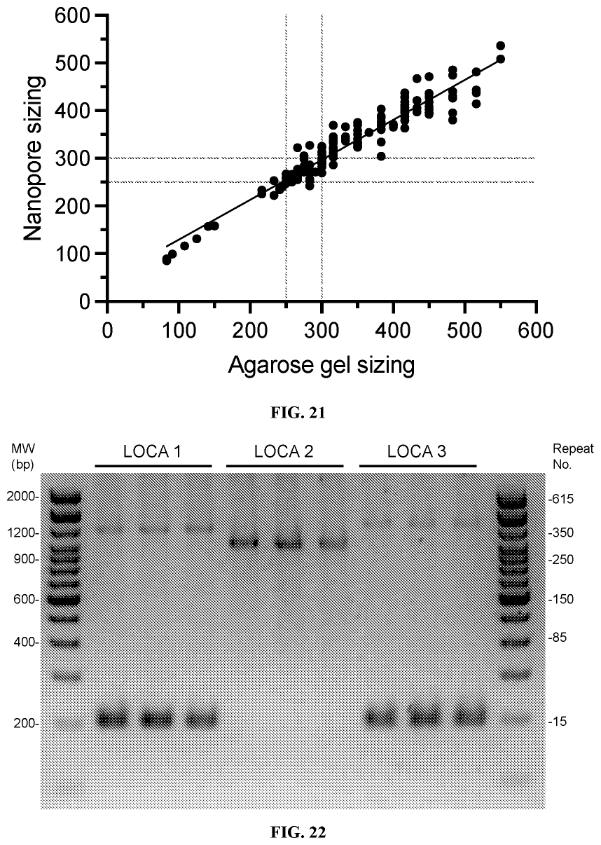

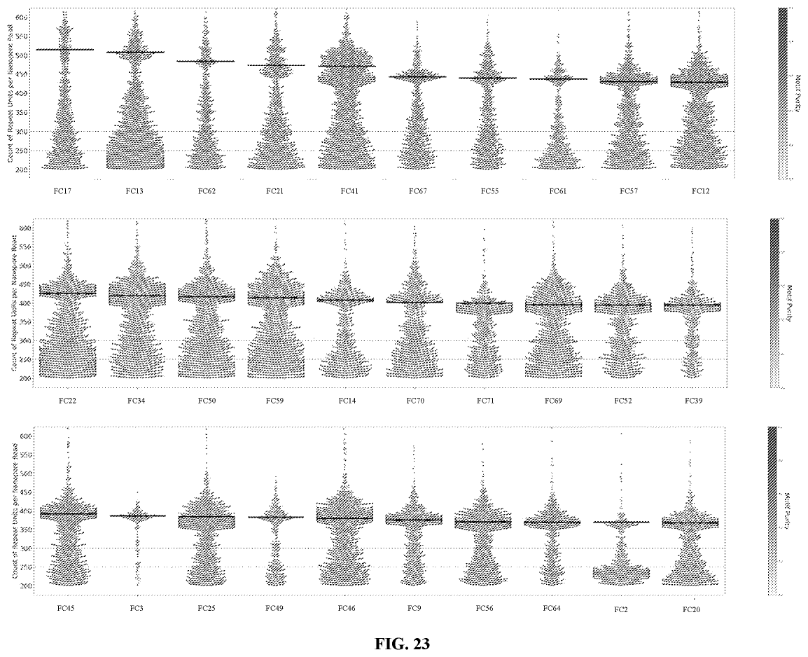

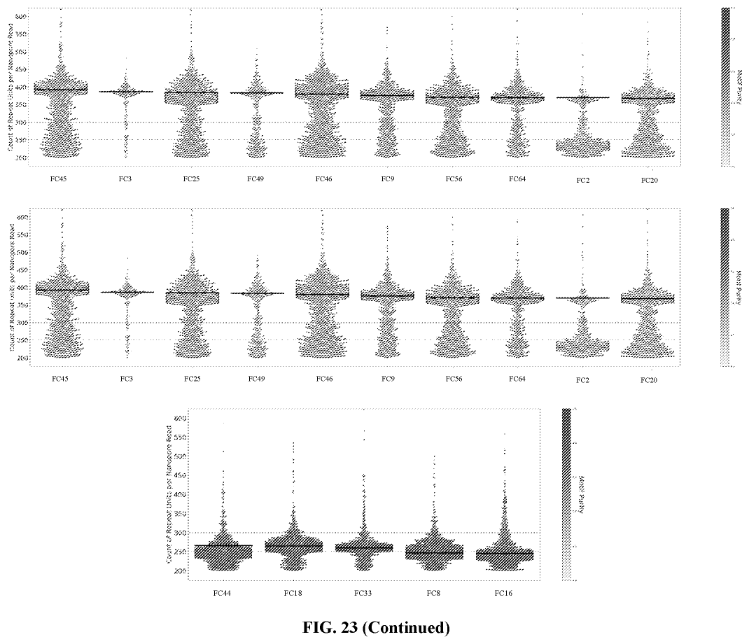

Analysis of the FGF14 Repeat Locus in Controls. The allelic distribution of the repeat locus was examined by means of long-range PCR in 408 controls (209 French Canadian and 199 German) and observed a wide variation in repeat sizes (FIGS. 2A and 18). Two thirds of alleles (544 of 816) carried 25 or fewer repeat units. Of the 816 control chromosomes, 11 carried an expansion ≥250 triplets, as assessed by PCR. Targeted long-read sequencing of these 11 alleles showed that 8 consisted of (GAA) n repeats, two of (GAAGGA) n repeats and one of [(GAA)₄ (GCA) 1]ₙ repeats. The allele frequency of (GAA)₂₅₀₋₃₀₀ alleles was 0.98% in controls and that none of the 816 control chromosomes surveyed carried (GAA)_{≥}300 expansions (FIGS. 19 and 20).

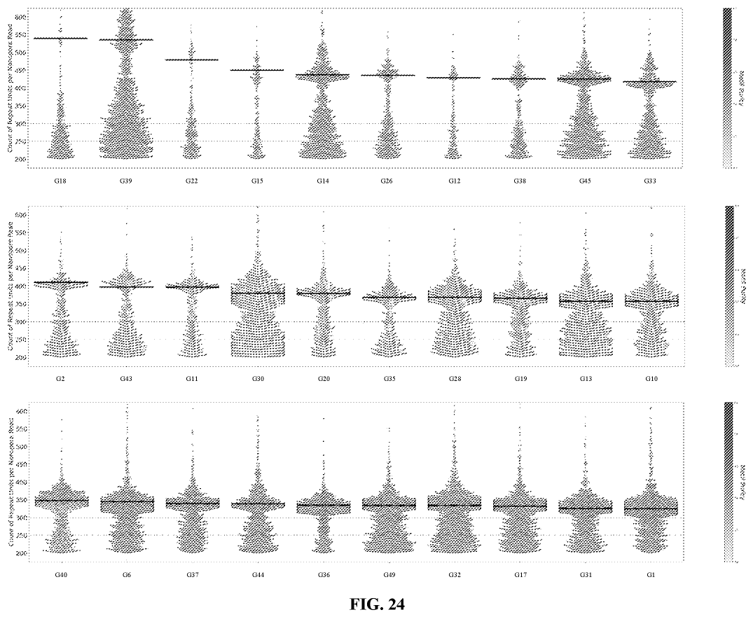

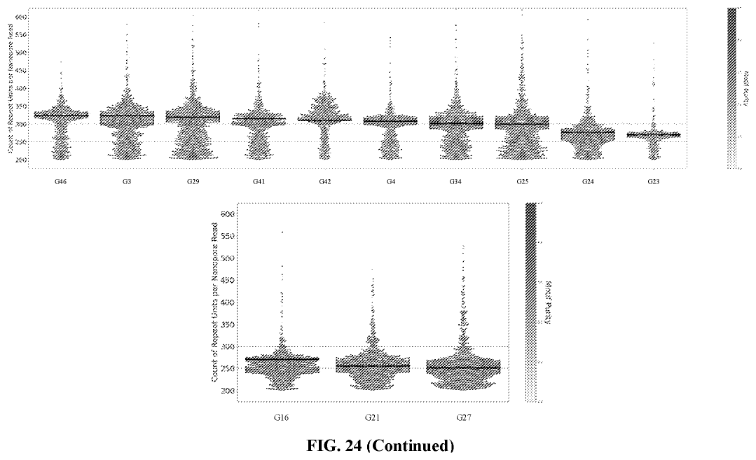

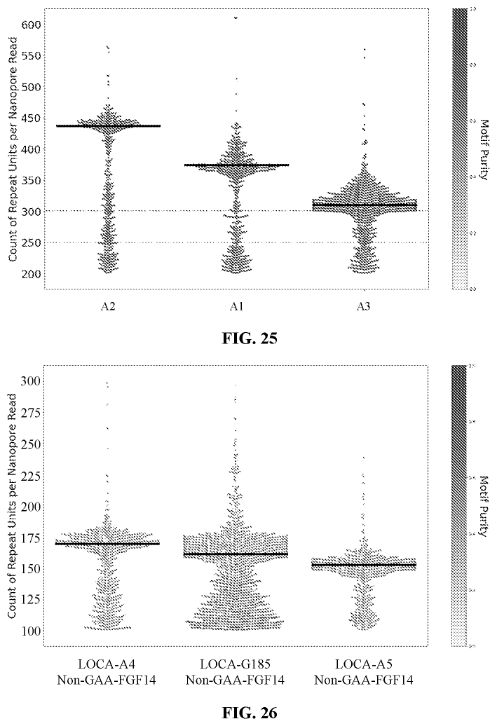

FGF14 GAA Repeat Expansion in Independent Cohorts of Patients with LOCA. Four independent cohorts of patients with unsolved LOCA from Quebec, Germany, Western Australia, and India were screened for the presence of the FGF14 repeat expansion (FIGS. 2B, 2C, 2D, 18, 24, 25, and 26). These cohorts consisted of 66 French Canadian index patients (excluding members of Families I, II, and III), 228 German index patients, 20 Australian index patients, and 31 Indian index patients.

Among the 66 French Canadian patients, 40 (61%) carried a (GAA)_{≥}250 expansion, as compared with 3 of 209 French Canadian controls (1%) (odds ratio, 105.60; 95% confidence interval [CI], 31.09 to 334.20; P<0.001 by Fisher's exact test of association). An FGF14 (GAA)≥250 expansion was also present in 12 affected relatives from nine families that included an index patient. The diagnostic yield of the repeat expansion in the French Canadian index cohort was 75% (24 of 32) among patients with early episodic features, 93% (26 of 28) among patients with downbeat nystagmus, and 94% (17 of 18) among patients with a combination of late-onset cerebellar ataxia, early episodic features, and downbeat nystagmus.

Of the 228 German patients, 42 (18%) carried a (GAA)_{≥}250 expansion, as compared with 5 of 199 (3%) German controls (odds ratio, 8.76; 95% CI, 3.45 to 20.84; P<0.001 by Fisher's exact test of association). The expansion was also present in 7 affected relatives in six German families that included an index patient. Furthermore, 3 of 20 Australian index patients (15%) and 3 of 31 Indian index patients (10%) carried a repeat expansion in FGF14 (FIG. 2D). A total of 128 patients (73 French Canadian, 49 German, 3 Australian, and 3 Indian) were found with an FGF14 (GAA)≥250 expansion and, accordingly, GAA-FGF14-related ataxia. Four of the patients were homozygous or compound heterozygous for (GAA)_{≥}250 expansions, and 2 were compound heterozygous for a (GAA)_{≥}250 expansion and a (GAAGGA)≥125 expansion. This supports that (GAA)₂₅₀₋₃₀₀ expansions are likely pathogenic albeit with reduced penetrance, while expansions (GAA)_{≥}300 are fully penetrant.

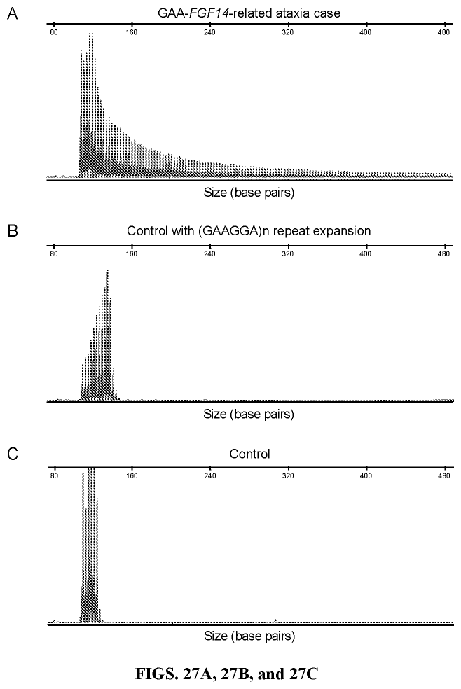

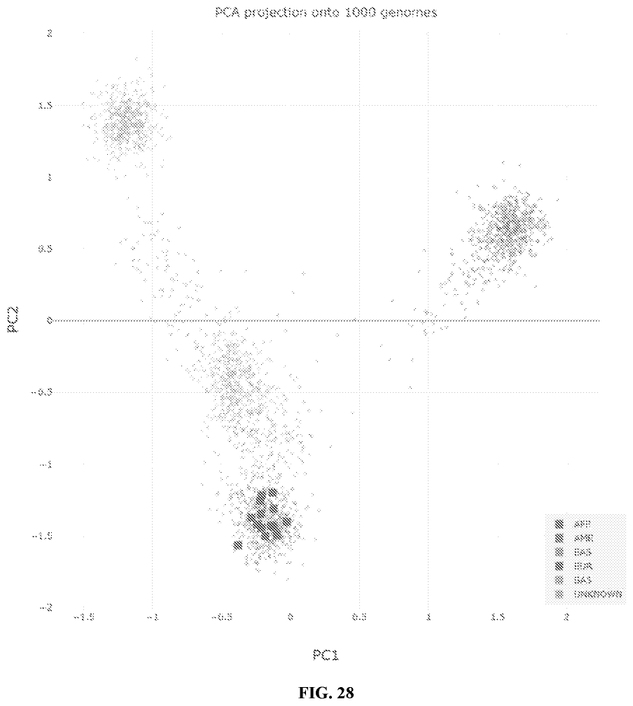

One German and two Australian patients carrying a (GAAGGA) n expansion were uncovered (FIGS. 27 and 28). The absence of segregation of this hexanucleotide repeat expansion with disease in affected family members from the two Australian families shows that non-GAA expansions at the FGF14 locus are not associated with LOCA.

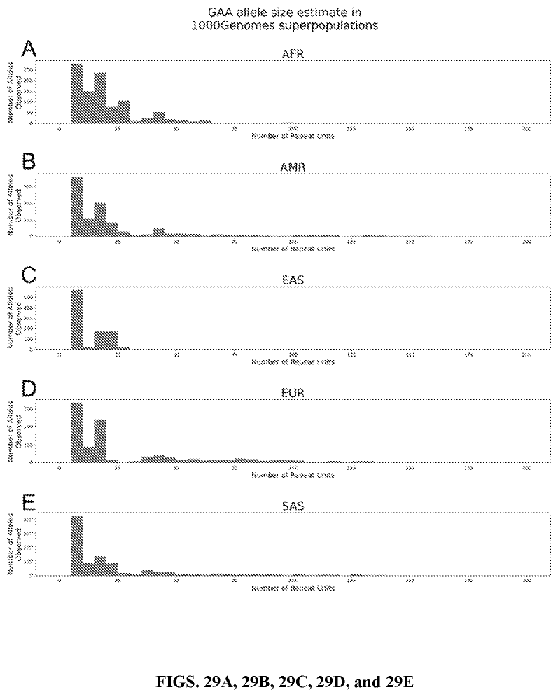

All but two patients with GAA-FGF14-related ataxia in the French Canadian, German, and Australian cohorts were of European ancestry (FIG. 29). The two non-European patients were from northeastern Turkey. All three Indian patients were of South Asian ancestry.

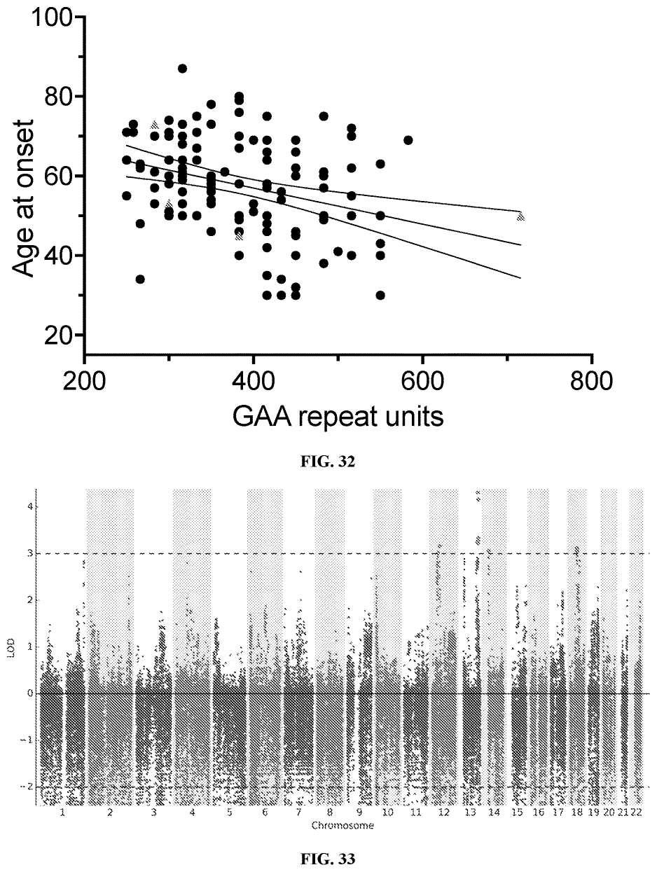

The high proportion of French Canadian patients carrying the FGF14 expansion correspond to a founder effect in this population, in which such effects are known to be common. In a finding consistent with this observation, a candidate 693-kb haplotype delimited by the polymorphisms rs12856547 and rs34644481 was shared by the 10 patients belonging to Family I and, at least in part, by the 4 other French Canadian patients for whom genome sequences were available (FIG. 32). The fact that GAA-FGF14-related ataxia was found in patients of Turkish and Indian descent shows that this repeat expansion may arise on multiple haplotype backgrounds.

Germline Instability of the GAA Repeat Expansion. The transmission of expanded (GAA)_{≥}250 alleles invariably resulted in expansion in the female germline and contraction in the male germline across 30 meiotic events examined (FIG. 2E). Four meiotic events in an affected father carrying (GAA) 300/716 expansions (Patient II.1, FIG. 1A) resulted in the inheritance of subpathogenic alleles (216, 225, 233, and 233 repeats) in the asymptomatic offspring. Reduced male transmission of the disease was further supported by the observation that only 20 of 66 familial cases (30%) were paternally inherited.

Clinical and Radiological Features. Table 1 summarizes the main clinical features of 122 of the 128 patients with confirmed GAA-FGF14-related ataxia. Overall, the ataxia was reported as episodic at onset in 46% of the patients. Episodes consisted of variable combinations of diplopia, vertigo, dysarthria, and appendicular and gait ataxia that lasted from minutes to days, with a daily to monthly occurrence. The most commonly reported triggers were alcohol intake and exercise. Two of the 47 French Canadian patients (4%) who presented with episodic disease initially received a misdiagnosis of acute peripheral vestibular syndrome. The mean (±SD) age at the onset of episodic symptoms and at the onset of progressive ataxia was 55±13 years (range, 30 to 87) and 59±11 years (range, 30 to 88), respectively. A weak inverse correlation was observed between the age at onset and the size of the repeat expansion (120 patients; Pearson correlation coefficient, −0.31; R²=0.10) (FIG. 33).

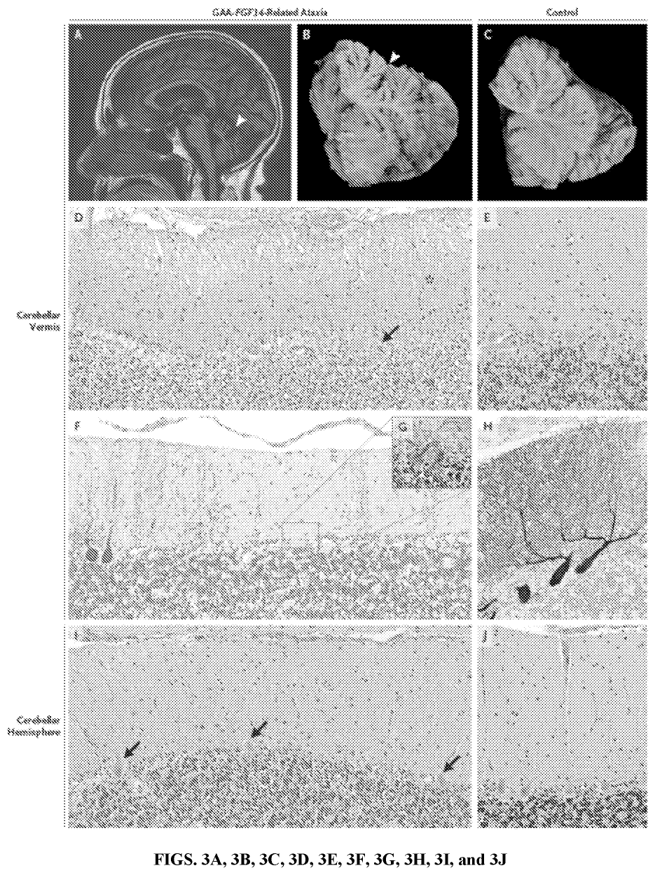

A substantial percentage of patients eventually used a walking aid (58%; 65 of 112), including the use of a wheelchair in 13% of patients (15 of 112). Downbeat nystagmus was observed in 42% of patients; this finding was especially prominent during episodes of ataxia. Vestibular areflexia was documented in 6 patients. Spasticity was found on examination in 8% of patients (9 of 120). Nerve conduction studies in 28 patients with suspected polyneuropathy on clinical examination revealed at most a mild peripheral axonal sensory neuropathy in 7 patients, which shows that polyneuropathy is not a core feature of GAA-FGF14-related ataxia. MRI of the head showed variable degrees of cerebellar atrophy in 74% of patients (67 of 91; FIG. 3A). Acetazolamide was mildly beneficial in 9 of 23 patients to whom it was given. In comparison, treatment with 4-aminopyridine resulted in a marked to moderate reduction in the frequency or severity of ataxic symptoms in 7 of 8 patients.

Neuropathological Examination. Neuropathological examination of the brain from two of the French Canadian patients revealed cerebellar atrophy that was more prominent in the vermis than in the hemispheres. Cerebellar atrophy was present in the form of widespread depletion of Purkinje cells, gliosis in the molecular layer, and overall mild cell loss in the granule-cell layer (FIGS. 3B, 3C, 3D, 3E, 3F, 3G, 3H, 3I, and 3J). Dentate nuclei showed no substantial atrophy, and the cerebellar white matter showed substantial pallor only in the subcortical regions of the vermis. No apparent atrophy in any region was seen on macroscopic and microscopic examination of brain stem and spinal cord. Immunostaining for p62 showed no evidence of pathological intranuclear or cytoplasmic inclusions in the cerebellum.

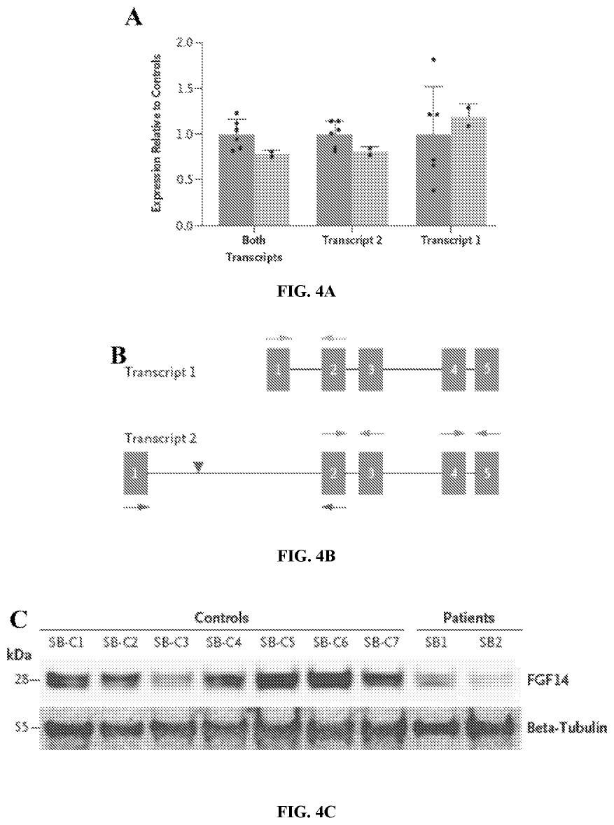

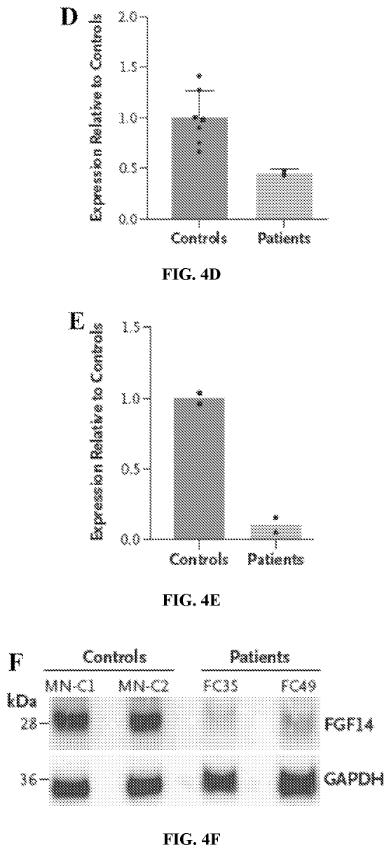

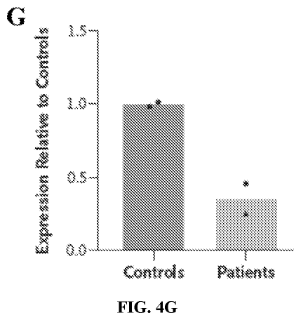

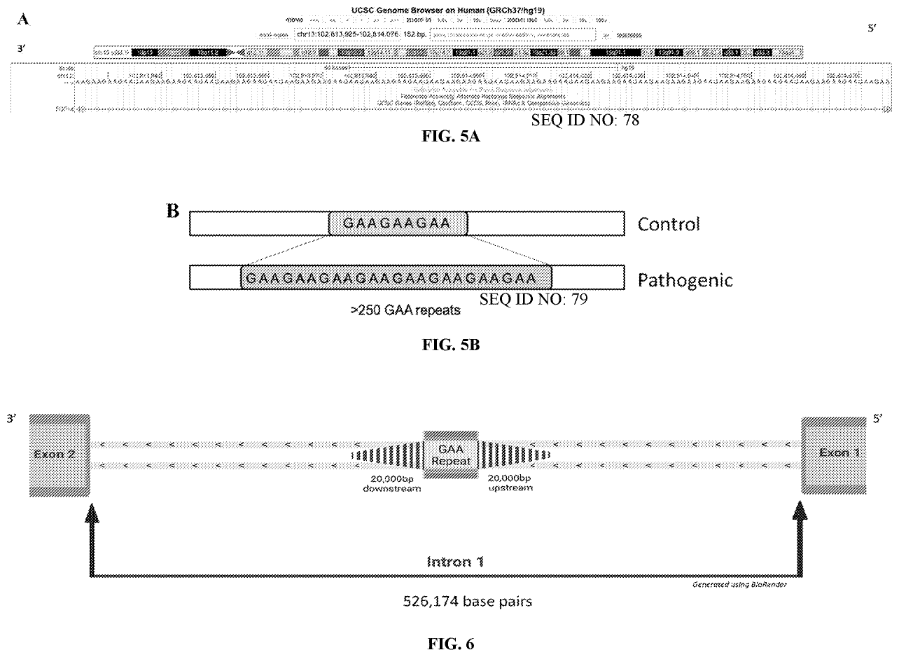

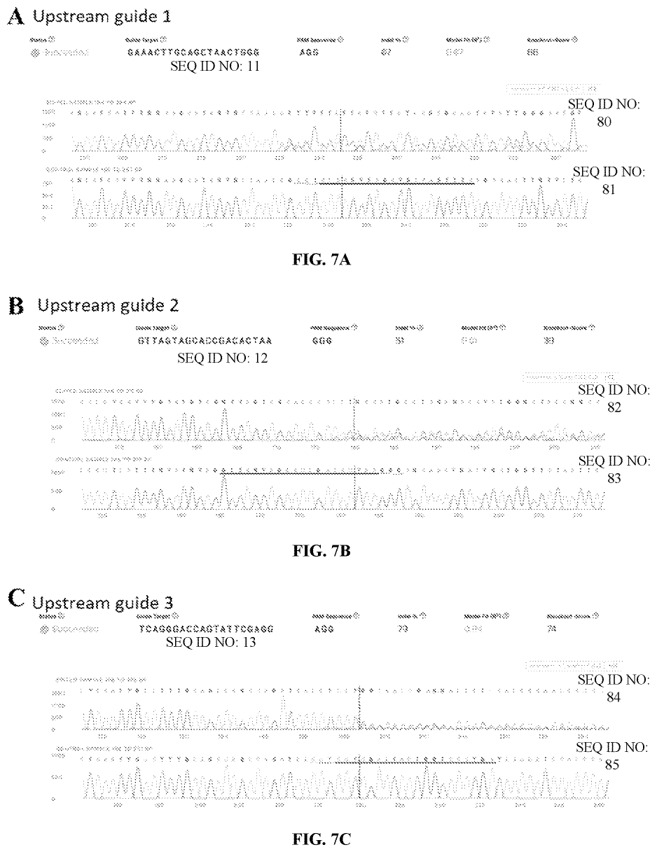

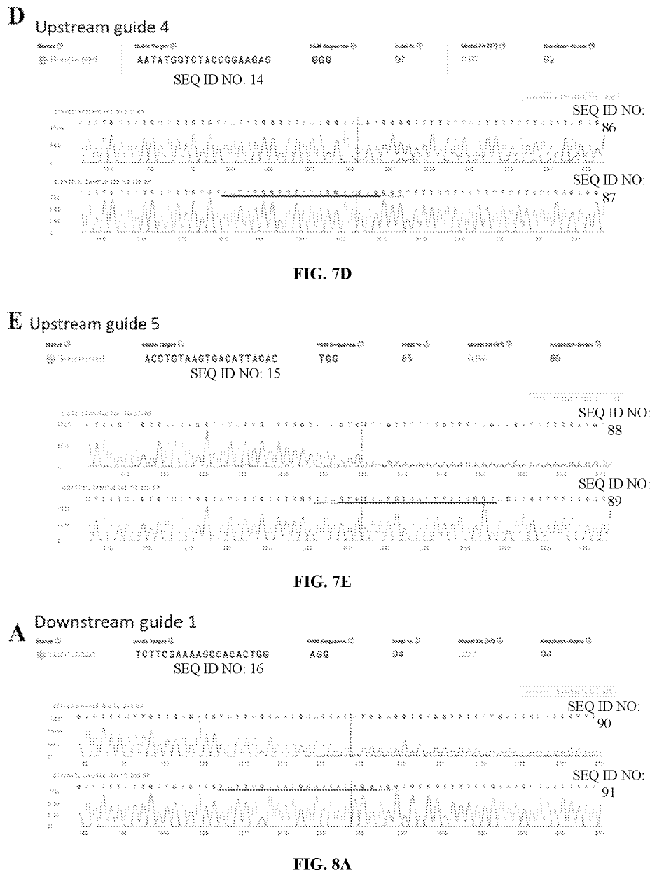

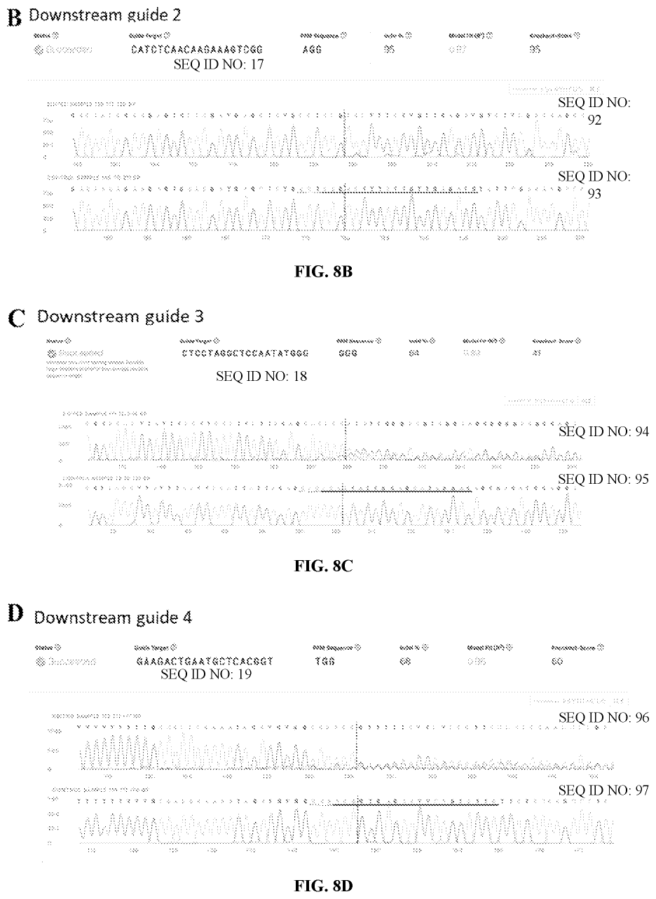

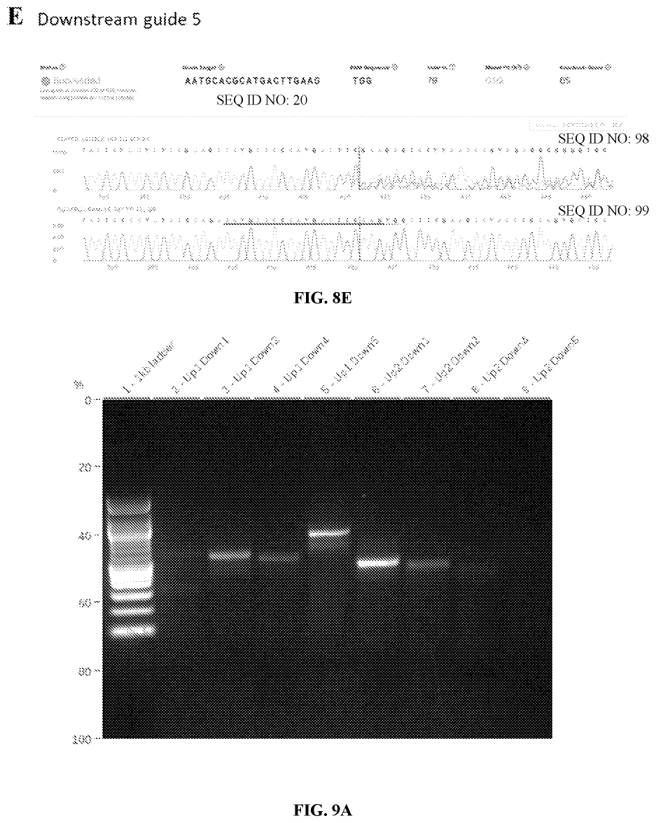

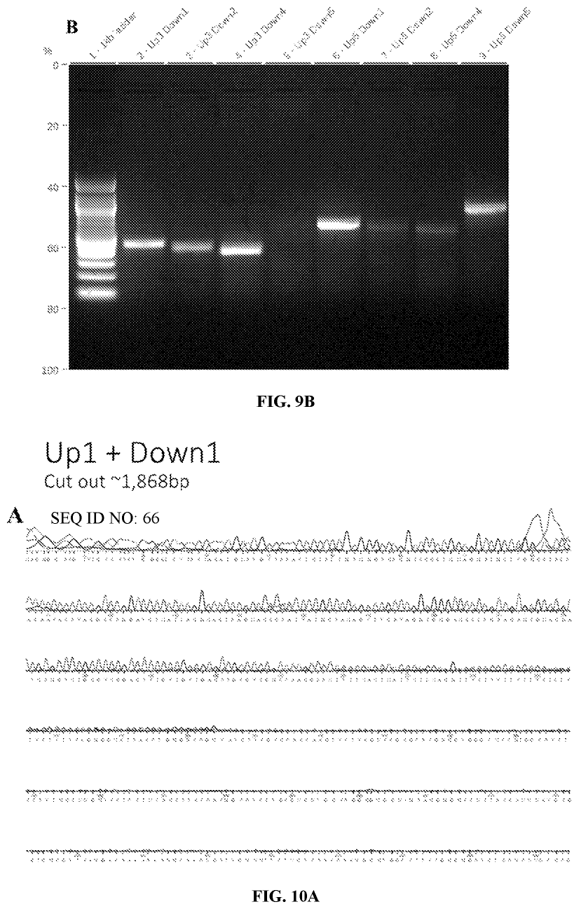

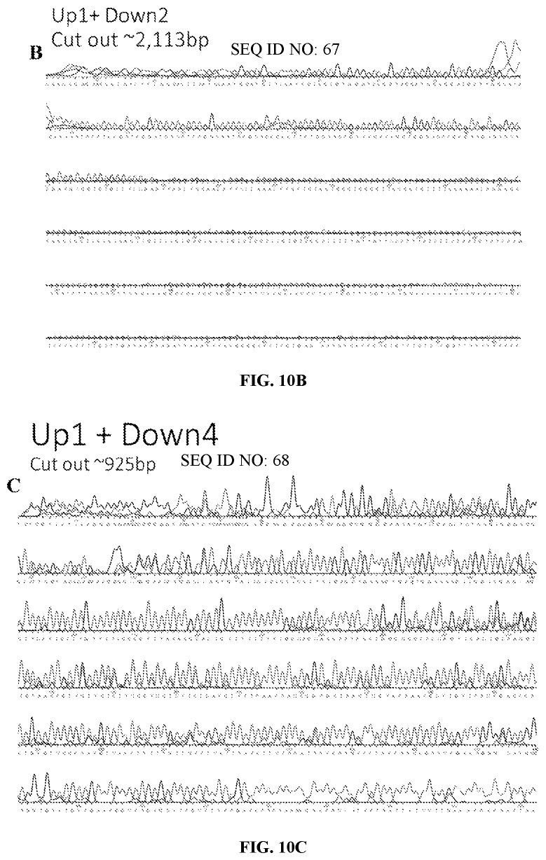

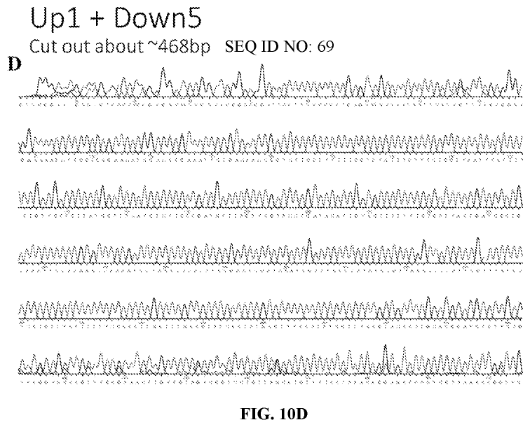

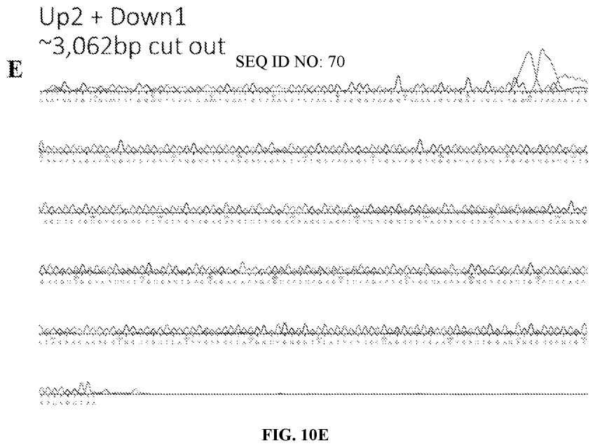

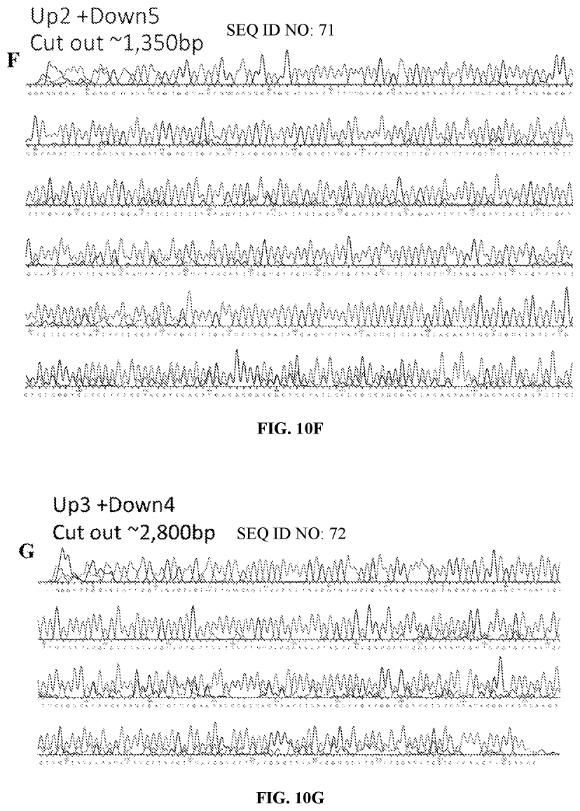

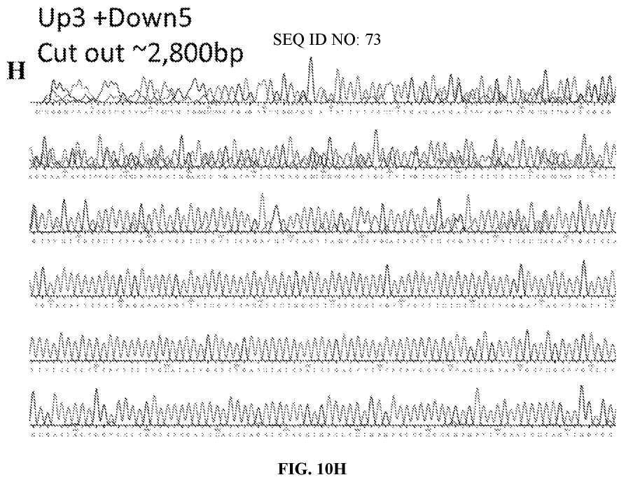

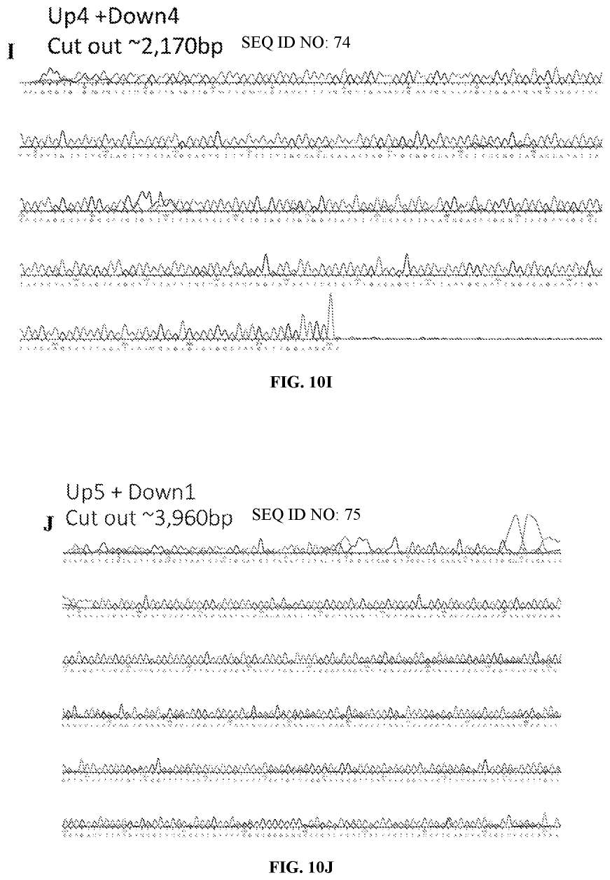

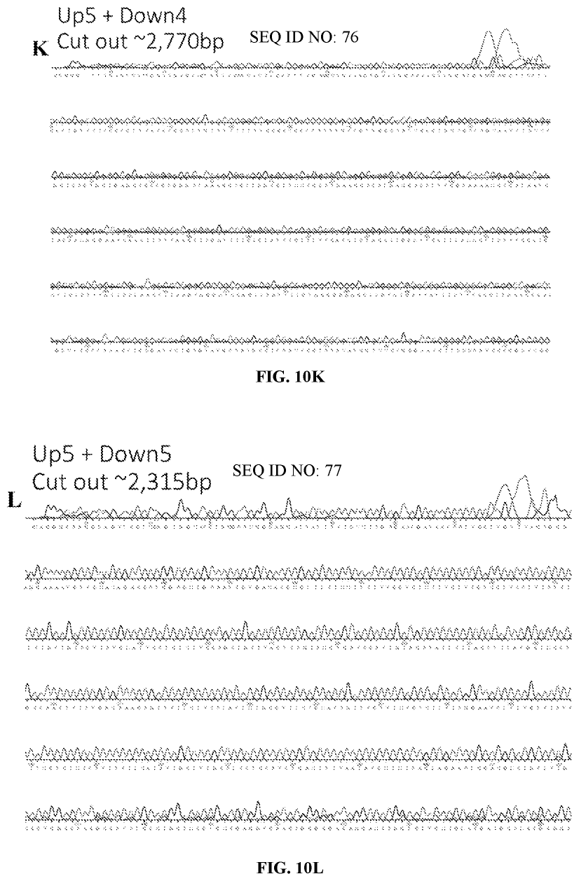

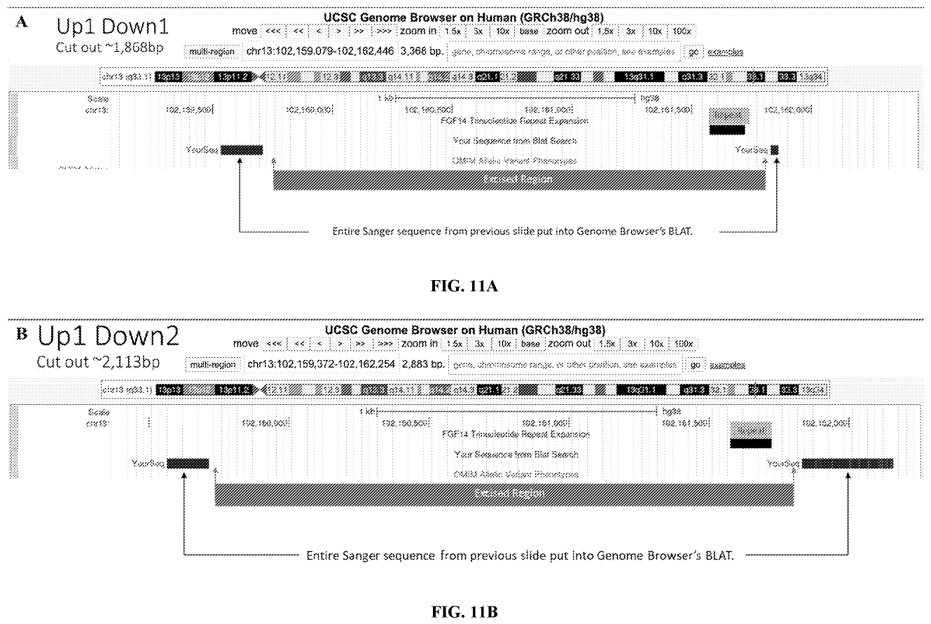

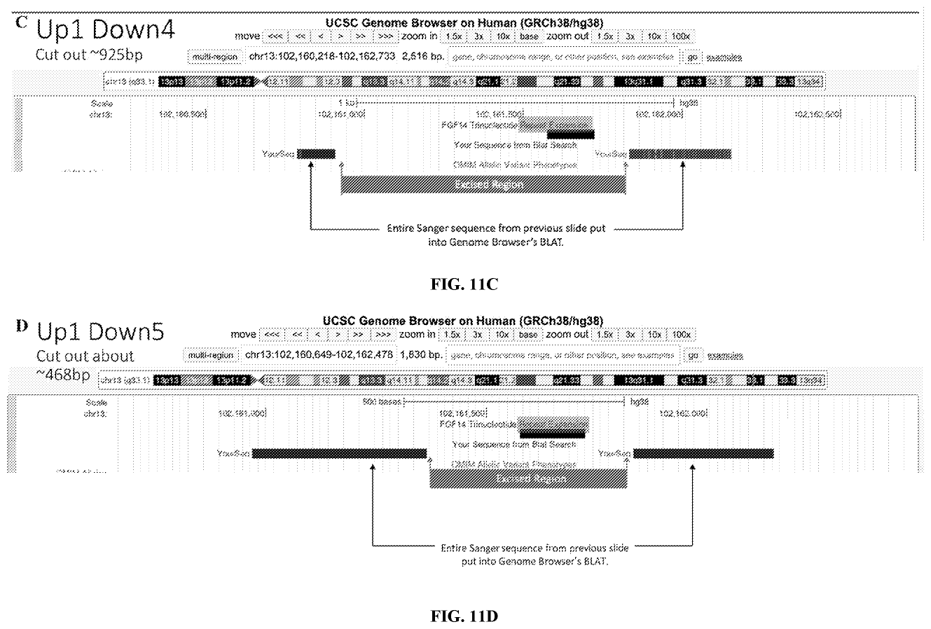

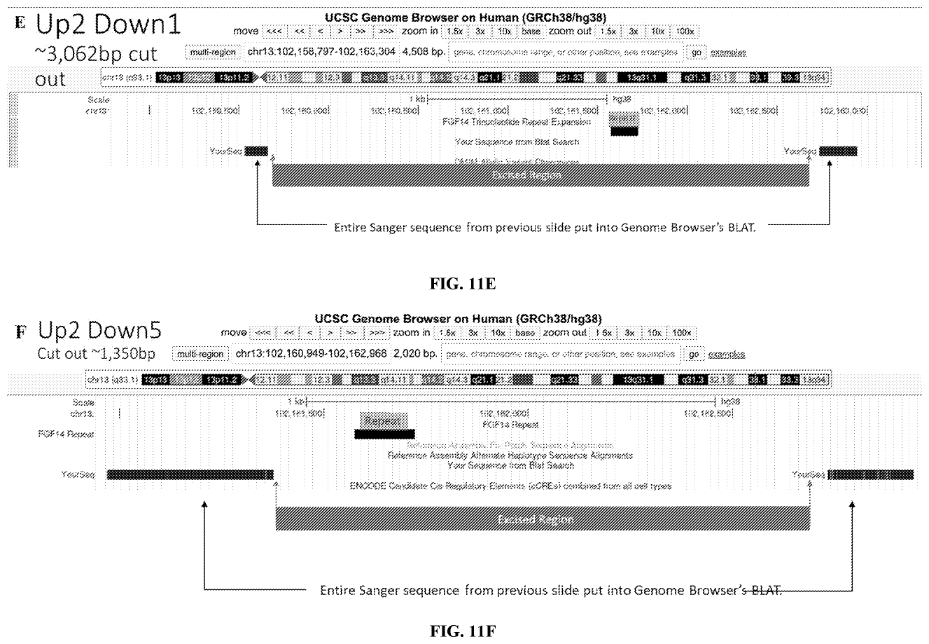

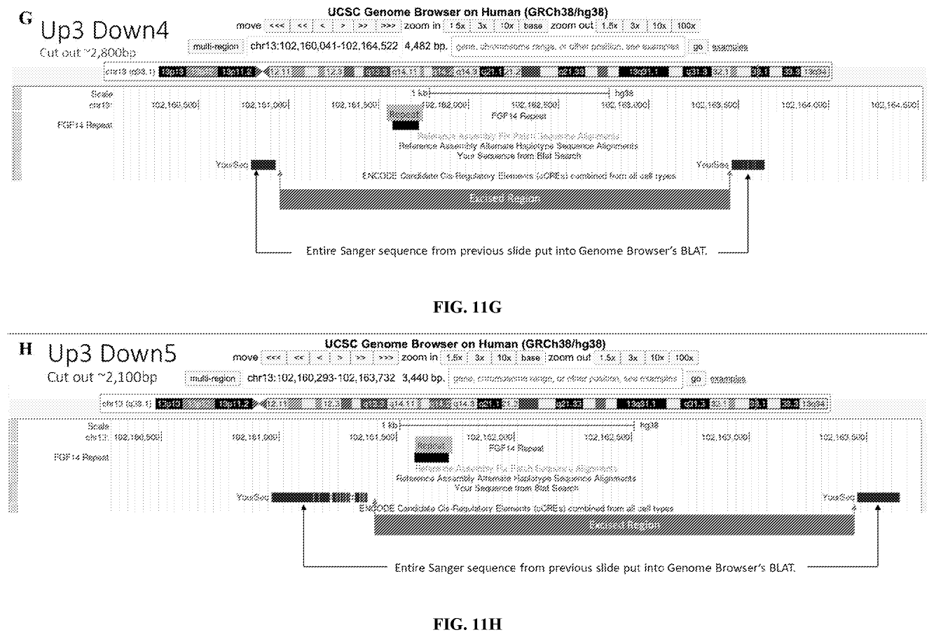

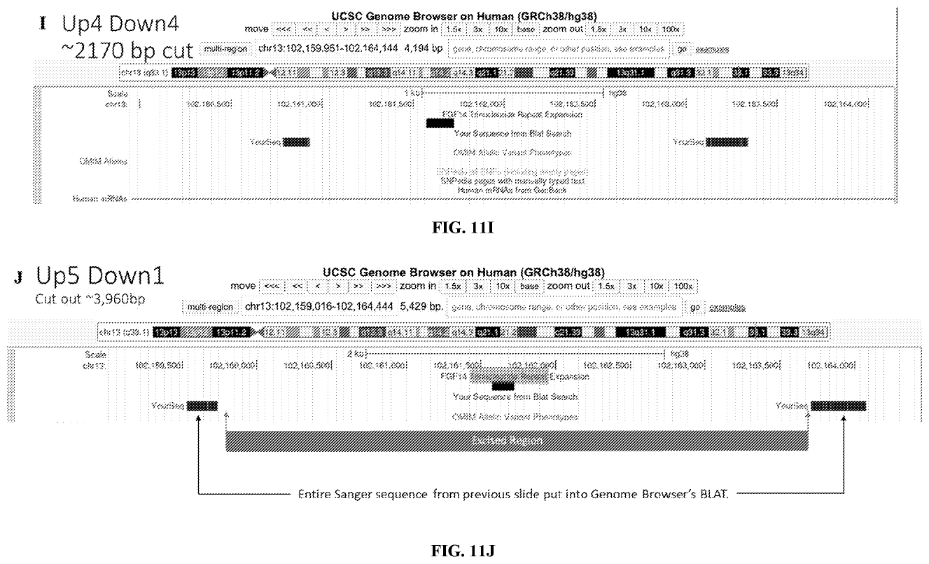

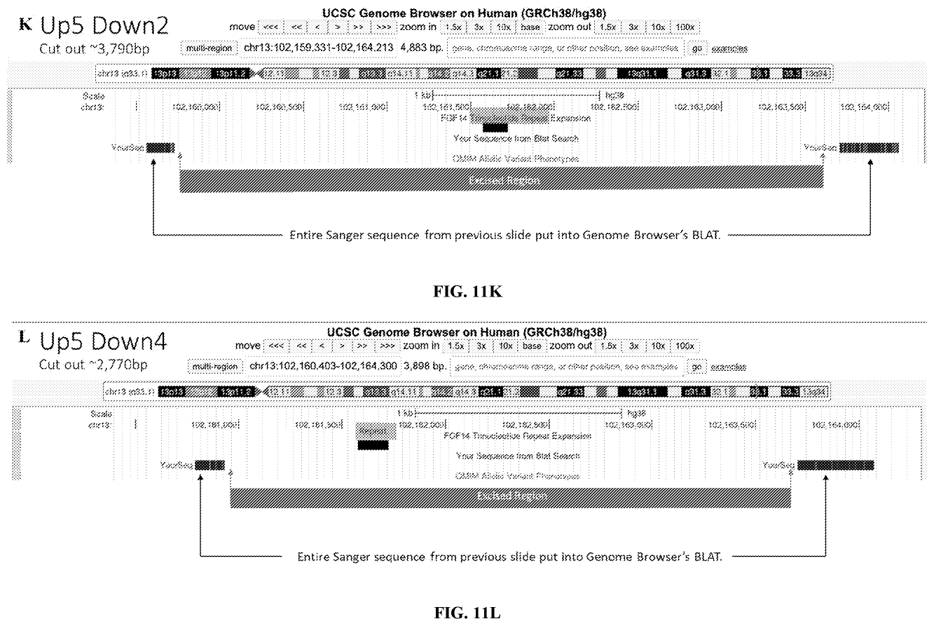

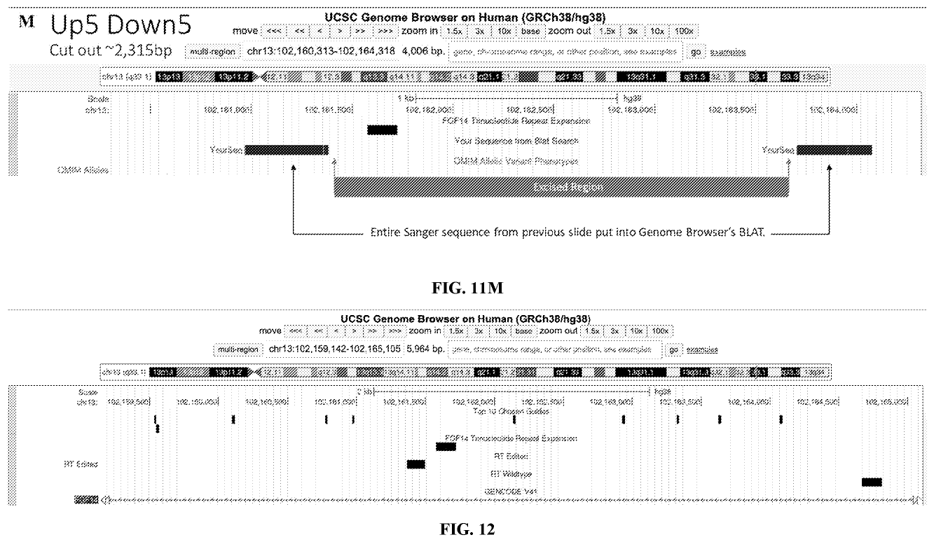

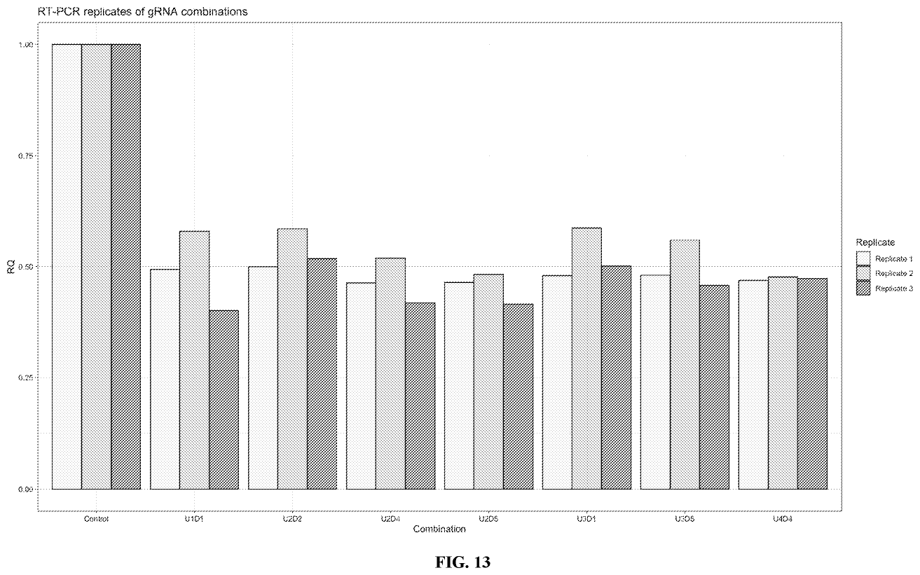

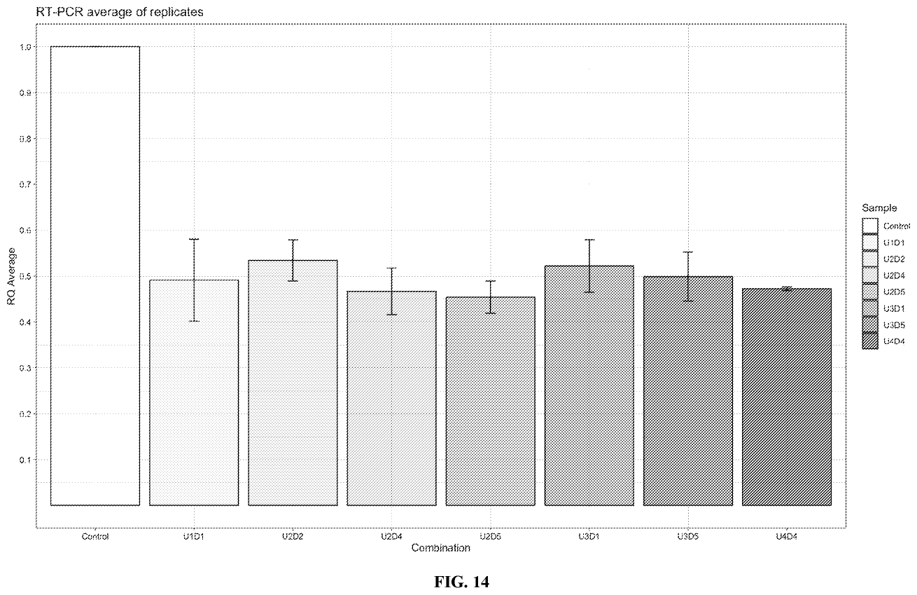

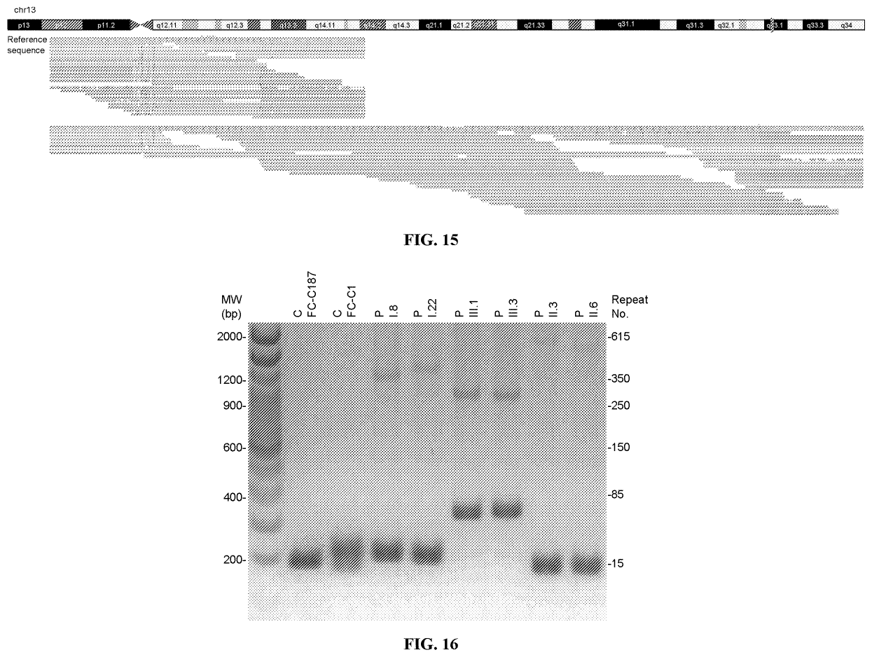

Mechanistic Studies. Postmortem cerebellum specimens and iPSC-derived motor neurons were used to investigate the mechanisms by which the GAA repeat expansion leads to pathogenicity. Two patients of Spanish origin who carried a GAA repeat expansion-Patient 1, carrying a (GAA) 300 expansion, and Patient 2, carrying a (GAA) 350 expansion-were found after screening of postmortem brains from 15 persons with unsolved LOCA. Both patients had late-onset episodic ataxia (ages at onset, 78 and 71 years) and downbeat nystagmus. Quantitative PCR showed 19.1% lower FGF14 transcript 2 expression among the patients than among controls (FIGS. 4A and 4B). The observation of a 22.0% lower total FGF14 expression among the patients provided further evidence for decreased transcript 2 expression, given that it normally has a higher expression than transcript 1 in the cerebellum.13 Immunoblotting showed 54.6% lower FGF14 levels in patient cerebellum relative to controls across triplicate experiments (FIGS. 4C and 4D).

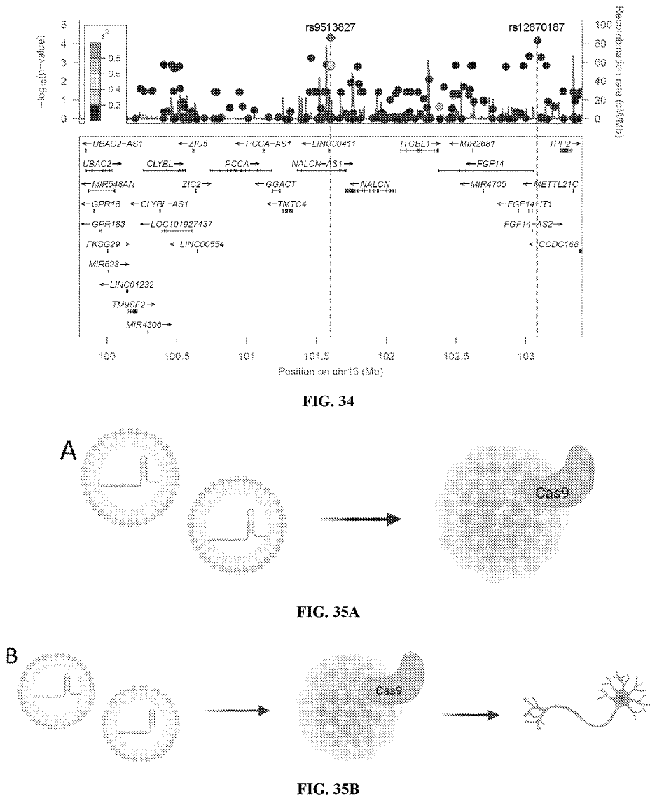

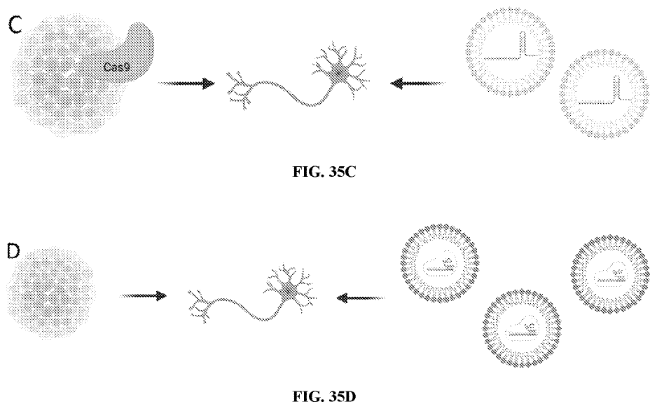

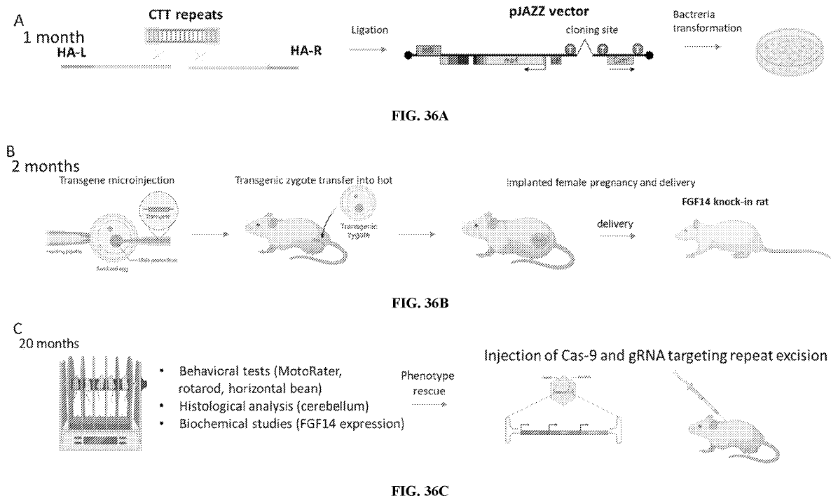

In the iPSC-derived motor neurons, levels of transcript 1 could not be reliably assessed by quantitative PCR because of the low level of expression. The level of transcript 2 expression was 84.3% lower in one patient and 94.2% lower in the other patient relative to controls (FIG. 4E). Consistent results were obtained in an assay of total FGF14 expression (FIG. 34). Western blot analysis showed FGF14 protein levels that were 54.3% lower in the first patient and 75.0% lower in the second patient than in controls (FIGS. 4F and 4G). The possibility of codominance of the FGF14 repeat expansion was suggested by the consistently lower levels of transcript 2 expression and FGF14 protein in the patient who was homozygous for FGF14 GAA expansions, as compared with the other patient, who was heterozygous. Taken together, data obtained from examinations of postmortem cerebellum and induced motor neurons show that the intronic GAA expansion in FGF14 leads to haploinsufficiency.

### DISCUSSION

A novel GAA repeat expansion was identified in the first intron of FGF14 in persons with LOCA. The FGF14 (GAA)_{≥}250 expansion is pathogenic according to criteria of the American College of Medical Genetics and Genomic because it is significantly more prevalent among affected persons than among controls (odds ratio >5.0, with a confidence interval around the estimate of odds ratio that does not include 1.0), it has a damaging effect on the gene product, and it cosegregates with disease in multiple affected family members. According to the ClinGen framework, strong support has been provided for pathogenicity of the FGF14 GAA repeat expansion. These results support incomplete penetrance of (GAA)₂₅₀₋₃₀₀ expansions and full penetrance of (GAA)_{≥}300 expansions.

Despite its prevalence in LOCA, the FGF14 GAA repeat expansion had, until now, resisted identification. Among contributing factors are the high degree of polymorphism of this repeat locus in the general population, the reduced male transmission of the disease, and the late onset of disease.

Several lines of evidence show that the FGF14 GAA repeat is highly unstable. First, the number of FGF14 GAA repeats varies from 8 to 300 in unaffected persons. In comparison, the FXN repeat length ranges from 5 to 37 repeats among unaffected persons. Second, although a common disease haplotype was shared by the 14 French Canadian patients from a haplotype analysis, the observation of the repeat expansion in Turkish and Indian patients shows that it arises on distinct haplotype backgrounds. Third, the relatively high incidence of sporadic GAA-FGF14-related ataxia may be a consequence of frequent stochastic expansion of the GAA repeat in the general population as a result of instability of intermediate alleles during parent-offspring transmission. Finally, large contractions of the GAA expansion in the male germline can lead to transmission of subpathogenic alleles to offspring and generation skipping of the disease. Contraction of repeat expansions during paternal meiosis has also been documented in Friedreich ataxia and other noncoding trinucleotide repeat expansion disorders.

Preliminary investigations of patient-derived postmortem cerebellum and induced motor neurons show that the intronic GAA expansion leads to loss of function by interfering with FGF14 transcription. This mechanism would be consistent with reports that SCA27 is caused by haploinsufficiency of FGF14, as well as with the high intolerance of FGF14 to loss of function (probability of being loss-of-function intolerant, 0.91; ratio of observed number to expected number of loss-of-function variants, 0.08; 90% CI, 0.03 to 0.39).

Loss of FGF14 function causes ataxia in Fgf14-knockout mice. FGF14 is expressed throughout the central nervous system, most abundantly in cerebellar granule and Purkinje cells. It is involved in spontaneous rhythmic firing of Purkinje cells by regulating and promoting localization of voltage-gated sodium channels at the axon initial segment. Impairment of ion channel kinetics, as previously shown with Fgf14 knockdown in mouse Purkinje cells, is consistent with the frequent episodic presentation in GAA-FGF14-related ataxia. These observations shows that GAA-FGF14-related ataxia is a type of channelopathy.

As compared with GAA-FGF14-related ataxia, SCA27 typically manifests at an earlier age (mean, 23.7 years), is frequently associated with early-onset postural tremor (96%) and neuropsychiatric manifestations (56%), and is less commonly associated with cerebellar atrophy (20%). Although this data support that most patients have a disease onset in the sixth decade of life, larger expansions being associated with ataxia occurring before 30 years of age cannot be ruled out. The association between a genetic marker in FGF14 (rs72665334, which is close to the repeat locus) and idiopathic downbeat nystagmus in a previous genome-wide association study is consistent with the common occurrence of downbeat nystagmus early in the disease course in patients with GAA-FGF14-related ataxia. The observation of vestibular areflexia in a subset of patients shows partial phenotypic overlap between GAA-FGF14-related ataxia and RFC1-related ataxia. GAA-FGF14-related ataxia can be distinguished from MSA-c, in which the age at onset is similar, on the basis of its frequent positive family history, slower progression, and, when present, isolated cerebellar atrophy on MRI.

In summary, a dominant GAA repeat expansion was identified in the first intron of FGF14 in persons with unsolved LOCA. These findings also show how late-onset disorders can be associated with individual alleles of strong genetic effect. This example underscores the importance of identifying noncoding repeat expansions, because they account for some of the missing heritability in unsolved neurodegenerative disorders.

### Example 3: Additional Methods and Results for Deep Intronic FGF14 GAA Repeat Expansion in Late-Onset Cerebellar Ataxia

Patient recruitment and institutional review board approval. Cases were recruited through ataxia clinics at the Montreal Neurological Hospital (Montreal, QC, Canada), the Centre Hospitalier Universitaire de Sherbrooke (Sherbrooke, QC, Canada), the Centre Hospitalier de l′Université de Montréal (Montreal, QC, Canada), the Center for Neurology, Tübingen (Germany), the Neurogenetics Clinic at Royal Perth Hospital (Australia), the Neurogenetics Clinic at the National Hospital for Neurology and Neurosurgery, London (United-Kingdom) and the National Institute of Mental Health and Neurosciences, Bengaluru (India). All participating subjects underwent clinical evaluation. Clinical data were collected through clinical assessment and review of medical records. Approval was received from the institutional review board of the Montreal Neurological Hospital (MPE-CUSM-15-915), the Centre Hospitalier de l′Université de Montréal (ND02.045), the Center for Neurology, Tübingen (598/2011BO1), the University of Western Australia (RA/4/20/1008), the UCL Queen Square Institute of Neurology, London (07/Q0512/26) and the National Institute of Mental Health and Neurosciences, Bengaluru (NIMHANS/25 IEC/2020-2021), and all patients provided written informed consent.

Snap-frozen cerebellar cortex from post-mortem brain of 15 patients with idiopathic late-onset cerebellar ataxia (LOCA) and seven neurologically healthy controls were obtained from the Queen Square Brain Bank for Neurological Disorders (six patients) and the Neurological Tissue Brain Bank of the Biobanc-Hospital Clínic-IDIBAPS Barcelona (nine patients and seven controls). Cases and controls were screened for FGF14 repeat expansion by long-range PCR following high-molecular-weight genomic DNA extraction using the MagAttract HMW DNA kit as per manufacturer's instructions (Qiagen). Repeat-primed PCR (RP-PCR) was used to confirm the GAA motif of the expansion. Controls were as much as possible, sex-, age- and post-mortem interval (PMI)—matched to the two genetically confirmed cases. None of the controls had evidence of cerebellar pathology on neuropathological examination. Details of the two genetically confirmed cases and seven neurologically healthy controls are provided in Table 4.

Fibroblast and lymphoblast cell culture. Skin biopsies were obtained in nine genetically confirmed French-Canadian LOCA cases carrying a ≥(GAA) 250 expansion and four healthy French-Canadian controls. Immortalized fibroblast cell lines from five additional healthy individuals were obtained through the Repository for Mutant Human Cell Strains of the McGill University Health Centre. Fibroblasts were cultured in DMEM, high glucose with 10% (v/v) fetal bovine serum, 100 U/ml penicillin-streptomycin, 2.5 μg/ml amphotericin B and 100 μg/ml L-glutamine. Immortalized lymphoblastoid cell lines were established from six genetically confirmed French-Canadian cases and six controls using standard methods. Cells were cultured in Iscove's modified Dulbecco's medium (IMDM) with 10% (v/v) fetal bovine serum, 100 U/ml penicillin-streptomycin, 2.5 μg/ml amphotericin B and 100 μg/ml L-glutamine. Cell lines were grown at 37° C., 5% CO₂ in 95% humidified air.

Neuronal Differentiation. Induced pluripotent stem cells (iPSCs) were differentiated from two patients carrying FGF14 GAA expansions and two controls. The two cases had a (GAA) 16/383 (case FC49) and a (GAA) 300/300 (case FC35) genotype, respectively. To enrich cultures with mature lower motor neurons, magnetic bead sorting using an anti-CD171PE antibody and anti-PE magnetic beads was used. CD171 is an axonal marker (LICAM) and is only expressed on mature neurons in differentiation cultures. Motor neurons were then cultured for seven days to allow neurite outgrowth and maturation prior to use in experiments. Immunocytochemistry (ICC) for several neuronal markers was performed to confirm successful generation of mature motor neurons. Chosen markers were Homeobox Protein 9 (HB9), Neurofilament Light Chain (NFL) and Beta-Tubulin 3 (BT3). Both patient iPSC-derived motor neuron lines stained positively for all three markers, consistent with control lines, confirming their neuronal lineage and therefore the ability to successfully generate and isolate motor neurons from the patient iPSC lines.

Whole-genome sequencing. Genomic DNA was isolated from peripheral blood of six French-Canadian LOCA cases using standard techniques. Whole-Genome Sequencing (WGS) was performed at the McGill Genome Centre. Samples were prepared using the TrueSeq PCR-free DNA library preparation kit and sequenced on the Illumina HiSeq X platform (Illumina) to generate 150 bp paired-end reads. The mean coverage was 30×. Sequences were aligned to the GRCh37 reference genome using the Burrows-Wheeler Aligner. Duplicate reads were marked using Picard's MarkDuplicates utility. Variant calling, annotation, and filtering was performed using the GATK best practices pipeline. Identification of candidate variants was based on: (1) segregation among all six cases; (2) minor allele frequency <0.0001 in gnomAD; (3) occurrence in known ataxia genes (as defined by the OMIM Catalog); (4) prediction of pathogenicity by in silico software; and (5) evolutionary sequence conservation.

In silico repeat expansion analysis. WGS data aligned to GRCh37 were further processed with ExpansionHunter Denovo version 0.6.2 with parameters set to restrict the search for large repetitive sites to motifs of length 3-8 nucleotides. The samples from the 1000 Genomes Project and the Vanderbilt Atrial Fibrillation Registry (VAFR) were processed as previously described. The six LOCA WGS samples were then compared as a group against each of the two control cohorts. The number of anchored in-repeat reads (IRRs) supporting each tandem repeat was compared among the groups with each sample normalized to 40× read coverage. For each comparison, a Cohen's d effect size was calculated. To identify tandem repeats expanded in the six cases relative to controls, the tandem repeats were filtered for a mean anchored IRR of at least five in cases (supporting the existence of a large tandem repeat >175 bp) and Cohen's d ≥1.3. Intergenic tandem repeats were excluded.

Linkage analysis and haplotype reconstruction. Following the discovery of the intronic GAA expansion, post-hoc two-point parametric linkage analysis was performed on the large French-Canadian Family I (FIG. 1A) using the mutation status as the phenotype. Prior to this discovery, previous linkage efforts had remained unyielding in this large family due to the uncertain or likely pre-symptomatic status of many of the younger subjects. Single-nucleotide polymorphism (SNP) genotype data were generated for 18 family members using the Illumina Infinium Global Screening Array-24 at the McGill Genome Centre. Individuals I.1, I.2, I.4, I.5, I.7, I.8, I.11, I. 12, I.13, I. 14, I. 15, I.16, I.17, I.18, I.22, I.28, I.38 and I.39 from Family I (FIG. 1A) were used for analysis. Analysis was performed using MLINK routine of FASTLINK v4.1P specifying a dominant disease with penetrance of 80% and disease allele frequency of 1:100,000. Graphical representation was done with Manhattan Generator v1.7.4. Regional representation was produced using LocusZoom. Haplotype reconstruction and phasing in Family I in the candidate region on chromosome 13 was carried out using SIMWALK version 2.90. The input files were converted to SIMWALK format using Mega2 v5.0.0. All 18 genotyped individuals from Family I were included for haplotype reconstruction.

Haplotype analysis. The 50 polymorphic sites composing the disease haplotype identified in Family I described above were then genotyped in four members of two additional French-Canadian families as well as one Australian case from whom WGS data were available. The genotypes of each of the 50 loci were inspected under the null hypothesis that all affected cases would share the same disease haplotype. At each locus, if the individual carried at least one copy of the polymorphism from the disease haplotype, then that locus did not support rejection of the null hypothesis in that individual. Only loci where neither allele matched the common polymorphism observed in the disease haplotype supported rejection of the null hypothesis for that individual.

Sanger sequencing. Sanger sequencing of PCR amplification products was performed at the Centre d′expertise et de services Génome Québec using the Applied Biosystems 3730xl DNA Analyzer (Applied Biosystems) to examine the tandem repeat motif at the FGF14 locus. Sequences were analyzed using SeqMan v.4.03 software (DNASTAR).

Repeat-primed polymerase chain reaction (RP-PCR). RP-PCR was used to assess for expansion of the GAA repeat tract in patients and controls. The primer sequences and experimental conditions are provided in Table 5. RP-PCR products analysis was performed on an ABI 3730x/DNA Analyzer (Applied Biosystems) using the GeneScan 1200 Liz Dye Size Standard (Applied Biosystems). Results were analyzed using the GeneMapper software (version 3.7, Applied Biosystems). The presence of characteristic saw-toothed products indicated the presence of a GAA pure expansion at the FGF14 repeat locus.

Nanopore sequencing. PCR amplification products were selected for molecular size >400 bp using SPRIselect paramagnetic beads for DNA size-selection following manufacturer's protocol (Beckman Coulter Life Sciences). Pre-sequencing size selection was performed to increase coverage depth of larger alleles. Amplicons were normalized to 150 ng/μl and then multiplexed using native barcoding expansion PCR-free library preparation kits and the SQK-LSK109 sequencing kit as per manufacturer's instructions, multiplexed and sequenced on the MinION or PromethION platform (Oxford Nanopore Technologies). The MinION run contained 23 natively barcoded samples and a libprep negative control. The R9.4.1 flow cell was run for 67 hours and generated 6.27 Gb of data with 18.8 million reads. The PromethION run contained 92 natively barcoded samples and a libprep negative control. The run was carried out on an R9.4.1 flow cell with 9,200 pores at run start. The run was stopped after 44 hours, when 53.13 Gb of data and 89.22 million reads were generated. Reads were base called and demultiplexed with stringent barcodes_both_ends setting using Guppy 5.0.13. Sequences were aligned to the GRCh37 reference human genome using Minimap2-2.17 with the predefined settings for nanopore data. STRique-v0.4.2 was then used to count the number of repeated units observed for each read spanning the FGF14 tandem repeat site. Motif purity was calculated for each sequencing read as the number of GAA units observed in the portion of the repeat locus-spanning segment of the read divided by the STRique estimation of the total number of repeat units for that read. Trinucleotide repeat size was estimated using a Gaussian mixture model. Polymorphic motifs of the repeat expansion locus detected by WGS in the 1000 Genomes Project cohort, namely GAAGGA, GAAGCA, GAAGAG, were looked for in samples with mean GAA purity <95%.

Quantitative reverse transcription PCR on human post-mortem cerebellum. Total RNA was extracted from human fibroblasts, lymphoblasts and post-mortem cerebellar cortex using TRIzol reagent as per manufacturer's protocol (catalog no. 15596026, Invitrogen). RNA integrity was assessed on a 2100 Bioanalyzer Instrument (Agilent Technologies). Complementary DNA (cDNA) was synthesized from DNase-treated RNA with the High Capacity cDNA Reverse Transcription Kit (catalog no. 4368813, Applied Biosystems). Quantitative PCR (qPCR) of postmortem brain samples was performed on a QuantStudio 7 Flex Real-Time PCR System (Applied Biosystems) using the TaqMan Fast Advanced assay (catalog no. 4444557, Applied Biosystems) and, for fibroblast- and lymphoblast-extracted RNA, on a QX200 droplet digital PCR System using the EvaGreen assay (catalog no. 1864034, Bio-Rad). For qPCR experiments on fibroblasts and lymphoblasts, samples were run in triplicate and FGF14 levels were normalized against expression of ACTB. Relative quantification was computed by the 2^{-ΔΔCt} method using the mean value of controls as calibrator. For qPCR experiments on post-mortem cerebellum, six controls and two patients were run in duplicate and FGF14 levels were normalized by geometric averaging of five stably expressed housekeeping genes (ACTB, HPRT1, YWHAZ, RPL13, UBE2D2) as ranked by Genorm. Data were analyzed with the QuantStudio Software v1.3 (Applied Biosystems). Primer sequences are provided in Table 6.

Quantitative reverse transcription PCR on iPSC-derived motor neurons. RNeasy mini kit (catalog no. 74106, Qiagen) was used to extract RNA from cultures of iPSC-derived motor neurons. Quantitect reverse transcription kit (catalog no. 205331, Qiagen) was used to convert 300 ng of RNA to cDNA. Next, the PowerUp SYBR green master mix (catalog no. A25741, Thermo Scientific) was used to set up the qPCR reactions in quadruplicates. 10 ng of cDNA was used per reaction with 400 nM of each of the forward and reverse primers. FGF14 levels were normalized against expression of GAPDH. Relative quantification was computed by the 2^{-ΔΔct} method using the mean value of controls as calibrator. Primer sequences are provided in Table 6.

RNA sequencing. RNA sequencing (RNAseq) was performed on patient (n=2) and control (n=2) fibroblast cell lines. RNA was prepared by ribodepletion using the TruSeq Stranded Total RNA with Ribo-Zero Gold library preparation (Illumina). Fibroblast samples were sequenced to 60 million 100 bp paired end read pairs per sample on a NovaSeq 6000 system (Illumina). RNAseq reads were aligned to the GRCh37 reference using STAR-2.7.7a with Ensembl release 75 being used to define the reference transcripts. Transcript abundances were quantified using RSEM in a strand-aware manner.

Immunoblotting on human post-mortem cerebellum. Post-mortem cerebellar tissues were homogenized in RIPA lysis buffer supplemented with EDTA-free protease inhibitor cocktail. Samples were next sonicated on ice and centrifuged for 25 min at 13,000 g at room temperature to remove cell debris. Protein lysate concentrations were measured with the Pierce BCA Protein Assay Kit (Thermo Scientific). Protein samples (15 μg) were mixed with Laemmli buffer containing dithiothreitol and heated at 70° C. for 10 min. Protein extracts were resolved by sodium dodecyl sulfate-polyacrylamide gel electrophoresis (4-12%) for one hour at 150V and transferred onto a nitrocellulose membrane for two hours at 85V at 4° C. After blocking for two hours at room temperature in 5% non-fat dry milk and 3% bovine serum albumin in Tris-buffered saline/0.1% Tween-20 (TBS-T), the membrane was incubated with rabbit polyclonal antibody anti-FGF14 (1:1,500; catalog no. PA5-77979, Invitrogen) or mouse monoclonal anti-beta tubulin (1:3,000; clone D3 U1W, catalog no. 86298, Cell Signaling Technology) overnight at 4° C. The membrane was then washed three times for five minutes with TBS-T and next incubated for one hour at room temperature with horseradish peroxidase-conjugated goat anti-rabbit IgG antibody (1:10,000; catalog no. 111-035-003, Jackson ImmunoResearch) or rabbit anti-mouse IgG antibody (1:10,000, catalog no. ab97046, Abcam). Blots were next washed three times in TBS-T and then developed using Amersham ECL Prime Western Blotting Detection reagents (catalog no. RPN2232, GE Healthcare) or SuperSignal West Femto Maximum Sensitivity Substrate (catalog no. 34095, Thermo Scientific) for fainter signal.

Immunobloting on iPSC-derived motor neurons. Proteins were extracted from cultures of iPSC-derived motor neurons by resuspending cell pellets in RIPA buffer containing protease inhibitor and incubated on ice for 5 minutes. Lysates were stored at −80° C. Lysates were sonicated and centrifuged at 13,000×g at 4° C. for 10 min to remove cell debris. Supernatant was collected and protein concentration was measured using the BCA kit (catalog no. 23225, Thermo Scientific). Cell lysates (25 ug) were run on a precast 4-12% Bis-Tris plus gel (catalog no. NW04125BOX, Invitrogen). The gel ran for one hour at 80 mV followed by 30 minutes at 100 mV. The gel was transferred to a PVDF membrane overnight at 4° C. The blot was then blocked in 5% non-fat dry milk with TBS-T for one hour on a shaker at room temperature. Next, the blot was incubated in a 1:1,000 solution of primary antibody (rabbit polyclonal anti-FGF14, catalog no. ab229610, Abcam; mouse monoclonal anti-GAPDH, clone 6C5, catalog no. 32233, Santa Cruz Biotechnology) in blocking buffer for two hours on a shaker at room temperature. After washing three times in TBS-T, the blot was incubated in a 1:2,000 solution of horseradish peroxidase-conjugated secondary antibody (goat anti-rabbit IgG, catalog no. 7074, Cell Signaling Technology; horse anti-mouse IgG, catalog no. 7076, Cell Signaling Technology) for one hour at room temperature on a shaker. Next, the blot was washed three times in TBS-T and then developed using SuperSignal West PICO PLUS kit (catalog no. 34580, Thermo Scientific). For fainter bands, SuperSignal West Femto (catalog no. 34094, Thermo Scientific) was used to amplify the signal. Densitometric analysis of signal intensity was performed using ImageJ.

Neuropathological examination. Postmortem brain tissue was available from two genetically confirmed French-Canadian female cases carrying a (GAA) 483 and (GAA) 516 expansion, respectively, who died at age 94 and 81, and had signed an informed consent for a post-mortem autopsy and brain tissue usage in medical research. Following brain removal, part of the brain tissue was frozen and the remainder after fixation in 10% buffered formalin were further dissected and processed for paraffin histology as per standard procedures. Sections of the frontal, temporal, parietal, occipital, primary motor cortices, anterior and posterior cingulate, amygdala, hippocampus, caudate, putamen, globus pallidus, hypothalamus at the level of the mammillary bodies, deep white matter, midbrain, pons, superior and inferior medulla, cervical, thoracic, and lumbar cord, and cerebellar vermis and lateral cerebellar hemisphere with dentate nuclei were sampled for histology. All sections were stained with routine hematoxylin and eosin, and specific regions were further examined with Luxol-fast blue and Bielschowsky tinctorial stains and immunohistochemistry. The sections were immunostained with anti-amyloid-β (1:50; catalog no. M0872, Agilent Technologies), anti-Tau (PHF-1) (1:100; from Dr. Peter Davies, Albert Einstein College of Medicine, New York, NY, USA), anti-4R-Tau (ET3) (1:200; from Dr. Peter Davies, Albert Einstein College of Medicine, New York, NY, USA), anti-α-synuclein (LB509) (1:200; catalog no. 180215, Thermo Fisher), anti-Glial Fibrillary Acidic Protein (GFAP) (1:500; catalog no. Z0334, Agilent Technologies), anti-p62 (1:100; catalog no. NBP1-49956, Novus Biologicals), and anti-calbindin (CB-955) (1:400; catalog no C9848, Sigma). Immunohistochemistry was performed using the Benchmark Ultra IHC platform with the OptiView DAB IHC detection system (Ventana Medical Systems Inc.) as per the published manufacturer specifications. Neuropathological examination was performed by two board certified neuropathologists.

### Results

Repeat motif purity of the FGF14 repeat locus. A recent genome-wide search of expanded tandem repeats in a large cohort of individuals with autism spectrum disorder (ASD) showed enrichment of polymorphic non-GAA repeat motifs at the same locus in FGF14 (chr13: 102,160,822-102,162,469, GRCh38) in this population compared to controls, suggesting that FGF14 may contribute to the complex heritability of ASD. Only seven of 17,231 genomes in the ASD cohort showed expansion of one of the following motifs: GAAGGA, GAAGAG, GAAAGAAGAA.

Expansion of the GAA repeat unit was, however, not reported in either the patient or control cohorts. The sequence polymorphism of the FGF14 repeat locus was characterized in the 1000 Genomes Project and Vanderbilt Atrial Fibrillation Registry (VAFR) control cohorts. 311 of 2,504 samples (12.42%) of the 1000 Genomes Project cohort and 301 of 1,115 samples (27.00%) of the VAFR cohort had evidence of at least five anchored in-repeat reads (IRR) using ExpansionHunter Denovo-v0.6.2, showing the existence of a repetitive region of >175 bp (~>58 trinucleotides). Among all samples showing >5 anchored IRRs, a pure GAA repeat motif was detected in the large majority (1000 Genomes Project cohort: 294/311 [94.53%]; VAFR cohort: 278/301 [92.36%]). Alternative motifs were detected with low frequency as shown in FIGS. 20A and 20B and included: GAAGGA, GAAGAG, GAAGCA and GCA. Two of these motifs (GAAGGA, GAAGAG) were previously reported by Trost et al.

The FGF14 repeat locus was analyzed for sequence variation by Sanger sequencing in 416 randomly sampled control and patient chromosomes of variable length as measured by long-range PCR. The repeat motif of some larger expansions could not be accurately determined due to limitations in Sanger sequencing of large alleles. Small alleles of <25 triplets were observed to all exhibit pure GAA repeat units. The smallest control alleles had 8 GAA repeat units. Intermediate size alleles of 25-249 triplets showed variable degrees of polymorphism. Alternative interspersions within the GAA sequence were observed in most intermediate alleles (107/136; 78.7%). The following interrupting motifs were observed: (CAA), (GAC), (GAG), (GCA), (GGA), (GAAA), (GGAA), (GAAGAG), (GAAGGA) or (GAAGAAA). Interruptions clustered at the 5′ end of the GAA repeat sequence in nearly 75% of chromosomes. The remaining alleles (29/136; 21.3%) showed pure and uninterrupted GAA repeat tracts. Alternative repetitive sequence motifs were observed in seven of 136 alleles (5.1%), and included (CAA) n, (GGA) n, (GAG) n, (GAAA) n and (GAAGGA) n. In comparison, expanded chromosomes above the pathogenic threshold of >250 triplets from LOCA patients and controls consisted of pure and uninterrupted GAA units (64/73; 87.7%), GAAGGA units (8/73; 10.9%) and [(GAA)₄ (GCA)₁] units (1/73; 1.4%) in the chromosomes studied.

Ancestry analysis of 14 affected individuals. Six GAA-FGF14-related ataxia cases from the French-Canadian cohort, seven GAA-FGF14-related ataxia cases from the German cohort and one GAA-FGF14-related ataxia case from the Australian cohort were assessed for ancestry and relatedness with one another. Four pairs of samples were already known to be related to each other—the six GAA-FGF14-related ataxia individuals from the French-Canadian cohort comprised three pairs of individuals from Families I, II and III; and one pair of individuals from the German cohort reported to be cousins. The 14 samples were analyzed with Peddy. Relatedness was supported for each of the four pairs of individuals, but no other combinations of individuals showed any evidence of being even distantly related to each other to the extent that is detectable with this method. Peddy was further used to inspect the ancestry of these individuals. All individuals self-reported to be of European ancestry, consistent with the Peddy analysis (FIG. 28). So, while all fourteen individuals with accessible relatedness and ancestry showed patterns consistent with European ancestry, they did not reveal any previously unknown or distant relations.

Analysis of size of FGF14 repeat locus in diverse control populations. Since the majority of GAA-FGF14-related ataxia cases in this example are of European ancestry, the distribution of sizes of this repeat were greater among European controls than among control individuals of other ancestries were also determined. For this analysis, Expansion Hunter calls were collected on each individual in the 1000 Genomes Project cohort. FIG. 29 shows histograms of the number of GAA repeat units observed in each individual stratified by superpopulation. This reveals that European and South Asian individuals do have alleles greater than the reference length of 50 repeat units more often than the other superpopulations. However, large alleles clearly exist in non-European populations as well.

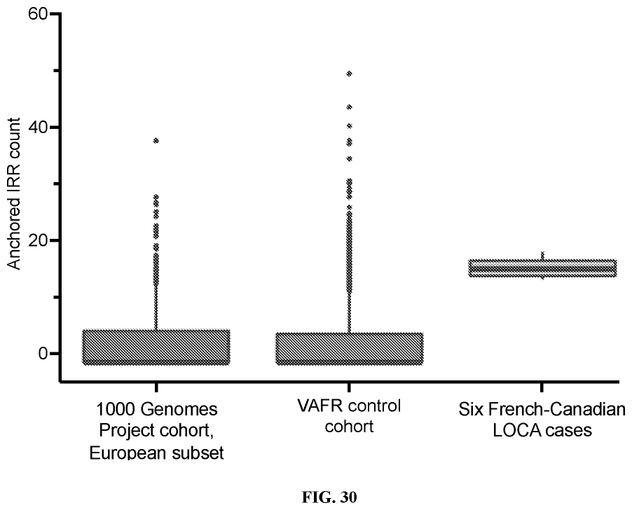

Considering these results, the analysis shown in FIG. 1C was repeated, but restricted the 1000 Genomes Project data to only the subset of individuals of European descent. FIG. 30 shows that the European subset of the 1000 Genomes Project dataset has a comparable distribution of anchored in-repeat reads to the VAFR cohort, which is also overwhelmingly of European descent. In this analysis, the Cohen's d value for enrichment in the six French-Canadian LOCA cases relative to the European 1000 Genomes Project samples was only 1.90, as opposed to 2.99 when including all 1000 Genomes Project populations.

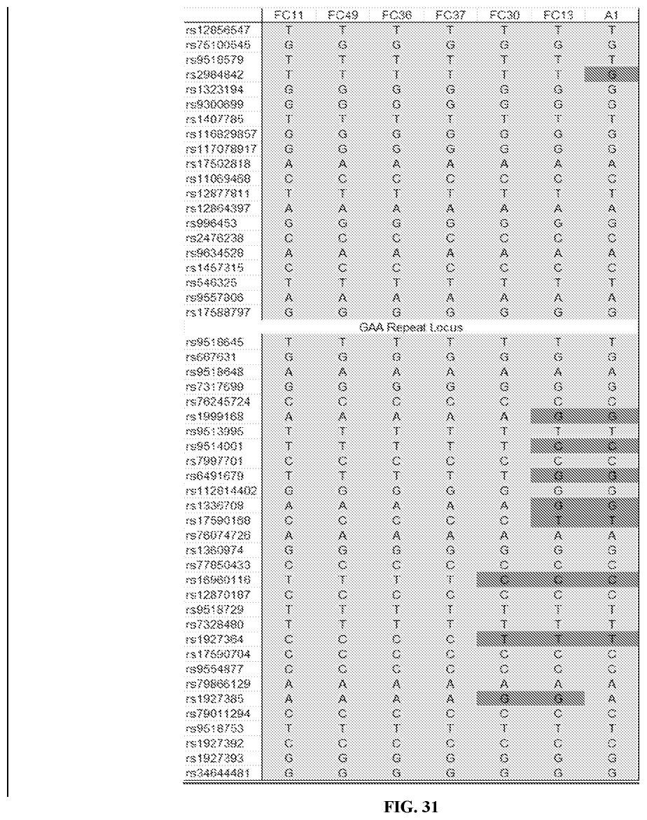

Shared disease haplotype analysis. Since the vast majority of cases found so far with GAA-FGF14-related ataxia are of European ancestry and particularly due to the high rate of observation within the French-Canadian LOCA cohort, evidence of a shared disease haplotype was explored among affected individuals. The linkage analysis from Family I was used to resolve a candidate 693 kb haplotype composed of 50 common polymorphic sites, the great majority of which are intronic. Examination of these polymorphic sites in four French-Canadian individuals from two unrelated families showed a shared haplotype among the cases, though with some recombination downstream of the repeat locus in two individuals (FIG. 31). This shared haplotype among French-Canadian cases is unsurprising given that population's relatively small set of founders. Analysis of these 50 sites in one member of the Australian cohort did not rule out the possibility of that individual sharing the haplotype as well. This analysis was not able to be performed on additional members of the French-Canadian cohort or members of the German cohort since only whole-exome sequencing data were available, which by in large did not cover haplotype-specific informative SNPs.

Neuropathologic examination. In addition to cerebellar cortical atrophy, in one of the patients (aged 94 years) post-mortem examination also revealed diffuse neocortical, Braak stage 6 Lewy body pathology, but with minimal neuronal depletion in the substantia nigra. Population-based autopsy studies of the prevalence of Lewy body disease shows that this represents concomitant independent age-related pathological process from GAA-FGF14-related ataxia. In this patient, also neurofibrillary tangle tau pathology, restricted to limbic regions and corresponding to Braak and Braak stage II was evident.

FGF14 is not expressed in fibroblasts and lymphoblasts. Real-time PCR and digital droplet PCR failed to detect expression of FGF14 in control and patient fibroblasts and lymphoblasts. This was confirmed by RNA sequencing (RNAseq) performed on fibroblasts derived from three controls and three patients. These results are consistent with the predominant expression of FGF14 in the central nervous system, more specifically in the cerebellum.

Linkage analysis. Post-hoc two-point parametric linkage analysis performed on the large French-Canadian Family I (see FIG. 1A) using the mutation status as the phenotype identified significant linkage loci with logarithm of the odds (LOD) score >3 on chromosomes 12, 13, 14, and 18 (FIG. 33). The highest LOD scores were obtained on chromosome 13 at SNP rs9513827 (LOD=4.31) and rs12870187 (LOD=4.16). This region fully encompasses FGF14 (FIG. 34).

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

SEQUENCES

| | | Sequence Name |
|---|---|---|
| | | and/or Sequence |
| SEQ ID NO | Sequence | Features |
| SEQ ID NO: | GAAACUUGCAGCUAACUGGG | Upstream gRNA #1 |
| 1 | | |
| SEQ ID NO: | GUUAGUAGCACCGACACUAA | Upstream gRNA #2 |
| 2 | | |
| SEQ ID NO: | UCAGGGACCAGUAUUCGAGG | Upstream gRNA #3 |
| 3 | | |
| SEQ ID NO: | AAUAUGGUCUACCGGAAGAG | Upstream gRNA #4 |
| 4 | | |
| SEQ ID NO: | ACCUGUAAGUGACAUUACAC | Upstream gRNA #5 |
| 5 | | |
| SEQ ID NO: | UCUUCGAAAAGCCACACUGG | Downstream gRNA #1 |
| 6 | | |
| SEQ ID NO: | CAUCUCAACAAGAAAGUCGG | Downstream gRNA #2 |
| 7 | | |
| SEQ ID NO: | CUCCUAGGCUCCAAUAUGGG | Downstream gRNA #3 |
| 8 | | |
| SEQ ID NO: | GAAGACUGAAUGCUCACGGU | Downstream gRNA #4 |
| 9 | | |
| SEQ ID NO: | AAUGCACGCAUGACUUGAAG | Downstream gRNA #5 |
| 10 | | |
| SEQ ID NO: | GAAACTTGCAGCTAACTGGG(AGG) | Upstream target DNA |
| 11 | | #1; parentheses contain |
| | | PAM sequence |
| SEQ ID NO: | GTTAGTAGCACCGACACTAA(GGG) | Upstream target DNA |
| 12 | | #2; parentheses contain |
| | | PAM sequence |
| SEQ ID NO: | TCAGGGACCAGTATTCGAGG(AGG) | Upstream target DNA |
| 13 | | #3; parentheses contain |
| | | PAM sequence |
| SEQ ID NO: | AATATGGTCTACCGGAAGAG(GGG) | Upstream target DNA |
| 14 | | #4; parentheses contain |
| | | PAM sequence |
| SEQ ID NO: | ACCTGTAAGTGACATTACAC(TGG) | Upstream target DNA |
| 15 | | #5; parentheses contain |
| | | PAM sequence |
| SEQ ID NO: | TCTTCGAAAAGCCACACTGG(AGG) | Downstream target |
| 16 | | DNA #1; parentheses |
| | | contain PAM sequence |
| SEQ ID NO: | CATCTCAACAAGAAAGTCGG(AGG) | Downstream target |
| 17 | | DNA #2; parentheses |
| | | contain PAM sequence |
| SEQ ID NO: | CTCCTAGGCTCCAATATGGG(GGG) | Downstream target |
| 18 | | DNA #3; parentheses |
| | | contain PAM sequence |
| SEQ ID NO: | GAAGACTGAATGCTCACGGT(TGG) | Downstream target |
| 19 | | DNA #4; parentheses |
| | | contain PAM sequence |
| SEQ ID NO: | AATGCACGCATGACTTGAAG(TGG) | Downstream target |
| 20 | | DNA #5; parentheses |
| | | contain PAM sequence |
| SEQ ID NO: | TGGAAATTTGCTCTGTGGGCT | Forward Primer |
| 21 | | chr13: 102161798- |
| | | 102162411 |
| SEQ ID NO: | CGTTCCCAATATAGTGCGCCT | Reverse Primer |
| 22 | | chr13: 102161798- |
| | | 102162411 |
| SEQ ID NO: | AACCCCTCTTCCGGTAGACC | Forward Primer |
| 23 | | chr13: 102162735- |
| | | 102163346 |
| SEQ ID NO: | AGCTGTTTCTCTGGGCCTCTCC | Reverse Primer |
| 24 | | chr13: 102162735- |
| | | 102163346 |
| SEQ ID NO: | TCCAGACTTTAAGTGGCCTCCA | Forward Primer |
| 25 | | chr13: 102163461- |
| | | 102163873 |
| SEQ ID NO: | GGAAGTTTAGAGCAAGTGGAAGG | Reverse Primer |
| 26 | | chr13: 102163461- |
| | | 102163873 |
| SEQ ID NO: | ACTTCCCCACCCTACACCCT | Forward Primer |
| 27 | | chr13: 102163112- |
| | | 102163711 |
| SEQ ID NO: | GCTTCCAAACATAGCCAGACTCT | Reverse Primer |
| 28 | | chr13: 102163112- |
| | | 102163711 |
| SEQ ID NO: | TGGTCATCAGTAAACACAGTGTC | Forward Primer |
| 29 | | chr13: 102163705- |
| | | 102164383 |
| SEQ ID NO: | GGGAAGTGACCAAGGGAGCC | Reverse Primer |
| 30 | | chr13: 102163705- |
| | | 102164383 |
| SEQ ID NO: | AGCATCACTAGAAATTCAGCCAA | Forward Primer |
| 31 | | chr13: 102159382- |
| | | 102159777 |
| SEQ ID NO: | TGGGATAGCAAACACGGCATCA | Reverse Primer |
| 32 | | chr13: 102159382- |
| | | 102159777 |
| SEQ ID NO: | TCTGAGGAAGCGTGGAGGATG | Forward Primer |
| 33 | | chr13: 102160004- |
| | | 102160248 |
| SEQ ID NO: | AGGTCCATTTACAGCTTCAGTCT | Reverse Primer |
| 34 | | chr13: 102160004- |
| | | 102160248 |
| SEQ ID NO: | ACATTCTGCCTTCCTCCATTGT | Forward Primer |
| 35 | | chr13: 102160458- |
| | | 102161107 |
| SEQ ID NO: | TCTCACACACCCGGCTCTCTG | Reverse Primer |
| 36 | | chr13: 102160458- |
| | | 102161107 |
| SEQ ID NO: | GGCAAGCTTACACTGACTGA | Forward Primer |
| 37 | | chr13: 102161367- |
| | | 102161495 |
| SEQ ID NO: | ACTGGAGTAACTGTGGCTGT | Reverse Primer |
| 38 | | chr13: 102161367- |
| | | 102161495 |
| SEQ ID NO: | CCAGCACTGTCCAATAGAACTTT | Forward Primer |
| 39 | | chr13: 102164666- |
| | | 102164812 |
| SEQ ID NO: | GGTAGTTAATGTCTGTGCTGTCC | Reverse Primer |
| 40 | | chr13: 102164666- |
| | | 102164812 |
| SEQ ID NO: | TGCAAATGAAGGAAAACTCTT | Long-range Forward |
| 41 | | Primer |
| SEQ ID NO: | CAATGATGAATTAAGCAGTTCC | Long-range Reverse |
| 42 | | Primer |
| SEQ ID NO: | TGCCCACATAGAGCTTAGTCT | Repeat Primed Forward |
| 43 | | Primer |
| SEQ ID NO: | CACGACGTTGTAAAACGACGAAGAAGAAGAA | Repeat Primed Reverse |
| 44 | GAAGAAGAA | Primer |
| SEQ ID NO: | FAM-CACGACGTTGTAAAACGAC | M13-FAM FAM = 5′ |
| 45 | | Carboxyfluorescein |
| SEQ ID NO: | ATTGGCAATGAGCGGTTC | ACTB Forward Primer |
| 46 | | |
| SEQ ID NO: | AGCCACATCGCTCAGACAC | GAPDH Forward |
| 47 | | Primer |
| SEQ ID NO: | TGATAGATCCATTCCTATGACTGTAGA | HPRT1 Forward |
| 48 | | Primer |
| SEQ ID NO: | GCAATTACTGAGAGACAACTTGACA | YWHAZ Forward |
| 49 | | Primer |
| SEQ ID NO: | CACCCAGGGAGCTGTTACTG | RPL13 Forward Primer |
| 50 | | |
| SEQ ID NO: | TTGAATGATCTGGCACGGGA | UBE2D2 Forward |
| 51 | | Primer |
| SEQ ID NO: | GCAAGCTATGAAAGGGAACAG | FGF14 Forward Primer |
| 52 | | |
| SEQ ID NO: | TGAAGGTAGTTTCGTGGATGC | ACTB Reverse Primer |
| 53 | | |
| SEQ ID NO: | GCCCAATACGACCAAATCC | GAPDH Reverse |
| 54 | | Primer |
| SEQ ID NO: | CAAGACATTCTTTCCAGTTAAAGTTG | HPRT1 Reverse Primer |
| 55 | | |
| SEQ ID NO: | TGGAAGGCCGGTTAATTTT | YWHAZ Reverse |
| 56 | | Primer |
| SEQ ID NO: | CAAGCAAGGTGTCATCGTGT | RPL13 Reverse Primer |
| 57 | | |
| SEQ ID NO: | GCCCCATTATTGTAGCTTGCC | UBE2D2 Reverse |
| 58 | | Primer |
| SEQ ID NO: | GTGCTTTTACTTGGCGTCAC | FGF14 Reverse Primer |
| 59 | | |
| SEQ ID NO: | TATTGCAGGCAAGGCTACTACTTG | FGF14 Forward Primer |
| 60 | | |
| SEQ ID NO: | CATATGCTGCAGTGTCTTTGTG | FGF14 Forward Primer |
| 61 | | |
| SEQ ID NO: | GCAACCTGGTGGATATCTTCTC | FGF14 Forward Primer |
| 62 | | |
| SEQ ID NO: | GTTTTCACTCCCTGGATGGCAAC | FGF14 Reverse Primer |
| 63 | | |
| SEQ ID NO: | GTAGTAGCCTTGCCTGCAATA | FGF14 Reverse Primer |
| 64 | | |
| SEQ ID NO: | ACCTGGTCACTATACCCTTGA | FGF14 Reverse Primer |
| 65 | | |
| SEQ ID NO: | CACGGCATGTTTGTATTTTGGCACTTAATCAAA | PCR Products from |
| 66 | TGCATGTTAAATTCTACTGTGGGTCAGGTACCA | Cas9-stable HEK cells |
| | TGCAGGATGCTTGGCATACAAAAATAAATAAG | transfected with dual- |
| | ATAGGATCTCATAAGGCAATAAGCACACAATT | guide RNA pairing, |
| | ATGCTACAGTTTGATAGGTGGGACAACAGAAG | Up1 + Down1 |
| | AGTGCACGATGAGCTCTGCCTCCGACTTTCTTG | |
| | TTGAGATGTCTTCGAAAAGCCACTTTTCACTCA | |
| | TTCTCCAGGTTTTTTTTTTTTTTTATTTTTTTTT | |
| | TAGGCCCATTAAATGGGGAAGGTATCTTTCTCA | |
| | AAAAACTTTTTGCTCCGGTTATATTCTACTTTA | |
| | TAAAGGAATATTCACCCCTCCTAGGGGCCCCT | |
| | GAAGGGGGTTCTTAAAACCCATCTGCTGGCCTT | |
| | TAGGAAAAAAAAAGGAAAACCTTTGGGGTTTT | |
| | CCTTAATAAAGGGGTGCATTTTTGGGGGATCTC | |
| | AGACCCAGAC | |
| SEQ ID NO: | CAACACGGAATGATGTATTTGGCACTTAATCA | PCR Products from |
| 67 | AATGCATGTTAAATTCTACTGTGGGTCAGGTAC | Cas9-stable HEK cells |
| | CATGCAGGATGCTTGGCATACAAAAATAAATA | transfected with dual- |
| | AGATAGGATCTCATAAGGCAATAAGCACACAA | guide RNA pairing, |
| | TTATGCTACAGTTTGATAGGTGGGACAACAGA | Up1+ Down2 |
| | AGAGTGCACGATGAGCTCTGCCTCCGAGTTAG | |
| | CTGCAACATTCCCAAACTTGTTCTAATCCCTCC | |
| | CCTTACCATCTTTTAAAAAACAGGAGATAACTC | |
| | CTAAAAAAACTTGTTAACTGACAAAATGTCTC | |
| | CCAAGTGTCCCATTTTTGTTGTTCCATTTATATT | |
| | CTAATCTATAAAGTAATATTAACACATCAGAA | |
| | AGGCTCATGGAGGTGTTTCTTGAAACCCTACTG | |
| | CTTACCTTAAGGATAAAAAAAAGAAAACAGTT | |
| | ATAATTTTCCTTGATAAAAAGATTAAATTTAGG | |
| | GCCATATAGTGAGCAATGTCATTCACTCATTCT | |
| | CCAGGTTTTTTTTTTT | |
| SEQ ID NO: | TCTCGTTAT- | PCR Products from |
| 68 | AAGGGAGAAGCCGGGGGTAAGATCTCAAAATT | Cas9-stable HEK cells |
| | GGCGAGGAAGGGCGGCCAGGGCGGTCGAATA | transfected with dual- |
| | TGTTTCAGTTCTCAGAGGGAGGGAATTCGTAC | guide RNA pairing,, |
| | ACAGAAAGGGC---GGGAATCTTCGCT- | Up1 + Down4 |
| | AGTGTATTCACACTACATTTTGAGCTGAAACTG | |
| | AATGGAAAAGTTGGATGAG- | |
| | AGCTTCATTCATTGTTTTACCACTTTATAAGCA | |
| | CTGTTTTTTTTTGCCACCAAAAATAAATGGGGC | |
| | CAACCAGTTAACTGCAAGGTTCAAAAACTTGT | |
| | TCTATTCCCTCCTCTTATCATCTTTTAAAAAAC | |
| | AGGAGATAACTCCTAAAAAAGCTTGTTAACTG | |
| | ACAAAATGTCTCCCAAATGTCAAATTTTTGTTG | |
| | TTTCATTTATATTCTAAATTATAAAGTAATATT | |
| | AACACATCAGAAATTT- | |
| | CATGGGAGGTGTTTCTTGAAACCCTACTGCTTA | |
| | CCTTTAAGATAAAAAAAAAGAAAAC- | |
| | ATTATAATTTTCCTTGAAAAAAAAATTAA | |
| SEQ ID NO: | CTCGGGAA-GCC- | PCR Products from |
| 69 | GTTACTCCAGTGGCAACTGGAACGGCAGGATT | Cas9-stable HEK cells |
| | ATTTTTCCCTTGAGAATGATAATAATTCATTGT | transfected with dual- |
| | TTAGAGGGAGGAAAATCTTCCACAGAACATGG | guide RNA pairing,, |
| | ACCTGAAATTCTGACAACCGCTCTTCCTTTTTT | Up1 + Down5 |
| | CCTCTCTTTCTTTCTCCTTAATTTATTTTTTGTTG | |
| | TACTTATGCATTCATCTCTTCAAGATGTTTACTT | |
| | AGTACCTGATACATTGTAGATATTTTCACATTA | |
| | ATGATCCATGTAAATTTTATGAGAAACAGTTTT | |
| | CCTTTACATTCTGCCTTCCTCCATTGTTTCTTTC | |
| | TCTCTCTTAGGAATACTTTGTTTATTTTCCTCTC | |
| | TATTTTTTCCATATTGATTTGAACTATCAATCTG | |
| | ACTTTTAATATCCTCTAAGCAGAAATGCACGCA | |
| | TGACTTGAGTTAGCTGCAAGTTTCAGAAACTTG | |
| | TTCTAGTCCCTCCTCTTAGCATCTTTTAAAAAA | |
| | CAGGAGATAACTCCTAAAAAAGCTTG | |
| SEQ ID NO: | CAATGGTGTTGTAATTTGGCACTTAATCAAATG | PCR Products from |
| 70 | CATGTTAAATTCTACTGTGGGTCAGGTACCATG | Cas9-stable HEK cells |
| | CAGGATGCTTGGCCATACAAAAATAAATAAGA | transfected with dual- |
| | TAGGATCTCATAAGGCAATAAGCACACAATTA | guide RNA pairing,, |
| | TGCTACAGTTTGATAGGTGGGACAACAGAAGA | Up2 + Down1 |
| | GTGCACGATGAGCTCTGCCTCCGACTTTCTTGT | |
| | TGAGATGTCTTCGAAAAGCCACACGTGTCGGT | |
| | GCTACTAACCTACAAATGAGGGGGACGCTGCT | |
| | AACCACTGCCAGTGACCAGAAAAAGAGCTACC | |
| | AGACTTCTAGTATCCTCCCAAAAAGGCTTCACT | |
| | GCACATACAATAGAAAAAACCTGGCAGCTCAT | |
| | TTCAACACAAGGCTGGGGTACATTTTCCACAG | |
| | CATTCAACTCACTGGAGAGGCCCAGAGAAACA | |
| | GCTAA | |
| SEQ ID NO: | CCAGGGAA- | PCR Products from |
| 71 | GCACGTTACTCCAGTGGCAACTGGGAACGGCA | Cas9-stable HEK cells |
| | GGATTATTTTTCCCTTGAGAATGATAATAATTC | transfected with dual- |
| | ATTGTTTAGAGGGAGGAAAATCTTCCACAGAA | guide RNA pairing,, |
| | CATGGACCTGAAATTCTGACAACCGCTCTTCCT | Up2 + Down5 |
| | TTTTTCCTCTCTTTCTTTCTCCTTAATTTATTTTT | |
| | TGTTGTACTTATGCATTCATCTCTTCAAGATGT | |
| | TTACTTAGTACCTGATACATTGTAGATATTTTC | |
| | ACATTAATGATCCATGTAAATTTTATGAGAAAC | |
| | AATTTTCCTTTACATTCTGCCTTCCTCCATTGTT | |
| | TCTTTCTCTCTCTTAGGAATACTTTGTTTATTTT | |
| | CCTCTCTATTTTTTCCATATTGATTTGAACTATC | |
| | AATATGACTTTTAATATCCTCTAAGCAGAAATG | |
| | CACGCATGACTTGGTGTCGGTGCTACTAACCTA | |
| | CATACAAATGAGGGGGACGCTGCTAACCACTG | |
| | CCAGGGACCAAAAAAAAAGCTACCAAACTTCT | |
| SEQ ID NO: | CCAGGGAA- | PCR Products from |
| 72 | GCACGTTACTCCAGTGGCAACTGGGAACGGCA | Cas9-stable HEK cells |
| | GGATTATTTTTCCCTTGAGAATGATAATAATTC | transfected with dual- |
| | ATTGTTTAGAGGGAGGAAAATCTTCCACAGAA | guide RNA pairing, |
| | CATGGACCTGAAATTCTGACAACCGCTCTTCCT | Up3 + Down4 |
| | TTTTTCCTCTCTTTCTTTCTCCTTAATTTATTTTT | |
| | TGTTGTACTTATGCATTCATCTCTTCAAGATGT | |
| | TTACTTAGTACCTGATACATTGTAGATATTTTC | |
| | ACATTAATGATCCATGTAAATTTTATGAGAAAC | |
| | AATTTTCCTTTACATTCTGCCTTCCTCCATTGTT | |
| | TCTTTCTCTCTCTTAGGAATACTTTGTTTATTTT | |
| | CCTCTCTATTTTTTCCATATTGATTTGAACTATC | |
| | AATATGACTTTTAATATCCTCTAAGCAGAAATG | |
| | CACGCATGACTTGGTGTCGGTGCTACTAACCTA | |
| | CATACAAATGAGGGGGACGCTGCTAACCACTG | |
| | CCAGGGACCAAAAAAAAAGCTACCAAACTTCT | |
| SEQ ID NO: | CCGGGGAAAACCCCGTTAACTCCAGTGGGCCA | PCR Products from |
| 73 | ACTGAGAACGGCCAGGATATTTTTCCCTTGAG | Cas9-stable HEK cells |
| | AATGACTATAGTTTCATCCGTTTGACCGGGACG | transfected with dual- |
| | AAAATCTATCCACGCAAACATGGAACCTGATA | guide RNA pairing, |
| | TTCTGACAGCCGTCTCTTCCTTTTGTCCTTCCTT | Up3 + Down5 |
| | TCTTTCTCCTCTAACCAATTGTTTGTTGTACTTA | |
| | TGCATTCATCTCTTCAAGATGTTTACTTAGTAC | |
| | CTGCATACATTGTAGATATTTTCACCATAATGA | |
| | TCCATGTAAATTTTATGAGAAACAGTTTTCCTT | |
| | TACATTCTGCCTTCCTCCATTGTTTCTTTCTCTC | |
| | TCTTAGGAATACTTTGTTTATTTTCCTCTCTATT | |
| | TTTTCCATATTGATTTGAACTATCAATATGACT | |
| | TTTAATATCCTCTAAGCAGAAATGCACGCATG | |
| | ACTTGCGAATACTGGTCCCTGATAACCATCCAC | |
| | CCAGATCAAAGTGACCTCTGAGAACCCCCCAG | |
| | AGATTTCAATCCTAGTTGGTGC | |
| SEQ ID NO: | AAAGGGTG- | PCR Products from |
| 74 | GTGAGTCTTTCGTTGAGTTGTAATTCAACACTA | Cas9-stable HEK cells |
| | CATTTTGAGCTGAAACTGAATGGAAAAGTTGG | transfected with dual- |
| | ATGAGGAGCTTCATTCATTGTTTTACCACTTTA | guide RNA pairing, |
| | TAAGCACTGTTTTTTTTTGCCACCAAAAATAGA | Up4 + Down4 |
| | TGTGGCCAACCTTCCGGTAGACCATATTACACA | |
| | AGGCATGGCATGTGTTTTTTAATACCTCTGTGC | |
| | TAGAGGATAATTTACAAAATAAAGGCACAAGG | |
| | GCTATGATAGCCCTAAACTATACAGTAAGCAT | |
| | TTATTTGTCACCACAGGTATAATTCATCTATGA | |
| | CAGGTTATTAATGGAATTGGACAGAAAATGTT | |
| | TACAAGTATTTACATTATATGAGAGTCTGGCTA | |
| | TGTTTGGAAGCAA | |
| SEQ ID NO: | CAATGCTCTGTA- | PCR Products from |
| 75 | CTTGGCACTTAATCAAATGCATGTTAAATTCTA | Cas9-stable HEK cells |
| | CTGTGGGTCAGGTACCATGCAGGATGCTTGGC | transfected with dual- |
| | CATACAAAAATAAATAAGATAGGATCTCATAA | guide RNA pairing, |
| | GGCAATAAGCACACAATTATGCTACAGTTTGA | Up5 + Down1 |
| | TAGGTGGGACAACAGAAGAGTGCACGATGAGC | |
| | TCTGCCTCCGACTTTCTTGTTGAGATGTCTTCG | |
| | AAAAGCCACACCACTGGACAAAGCAATATGCC | |
| | TGGTCACTGCTGAGGTCAACAGGGAGAAAGGA | |
| | CATGGTCTTCCTGCATGAAGGACATGAGGACA | |
| | TTTGGTAAAACGTGATAATCTACAACGGGAAT | |
| | CATATTTTTAAGTTAGGTTTTCACTATTTCTTTT | |
| | TCATGTCTGGAATGGCTTCATTAACCCATCTTT | |
| | CCATGCTTCACTTTGTTCCAGACTTTAAGTGGC | |
| | CTCCAGAATGTTTTCGGGGGGGGGCAAGTCTG | |
| | ATTATTTCTTTACCTTCAACTAAAGCCTTCAAA | |
| | AA | |
| SEQ ID NO: | CCGGG- | PCR Products from |
| 76 | TTTGAGTCTTTCGTTGAGTTGTAATTCAACACT | Cas9-stable HEK cells |
| | ACATTTTGAGGCTGAAACTGAATGGAAAAGTT | transfected with dual- |
| | GGATGAGCGAGCTTCATTCATTGTTTTACCACT | guide RNA pairing, |
| | TTATAAGCACTGTTTTTTTTTGCCACCAAAAAT | Up5 + Down4 |
| | AGATGTGGCCAACCCACTGGACAAAGCAATAT | |
| | GCCTGGTCACTGCTGAGGTCAACAGGGAAAAA | |
| | GGACATGGTCTTCCTGCATGAAGGACATGAGG | |
| | ACATTTGGAAAAACGTGATAATCTACAACGGG | |
| | AATCAAATTTTTAAGTTAGGTTTTCACTATTTG | |
| | TTTTTCATGTCTGGAATGGCTTCATTAACCCTT | |
| | CTTTCCATGCTTCACTTTGTTCCAAACTTTAAGT | |
| | GGCCTCCAGAATGTTTTCTGGGGGGGGCAAGT | |
| | CTGATTATTTCTTTACCTTGAACTAAAGCCTTC | |
| | AAAAACTTCCCATTCTGTGTATGAAATGCCTAA | |
| | ACTCCTAAACATGGTCCCTGCGAATCTTCCCAA | |
| | TCCAGGCTCCC | |
| SEQ ID NO: | ACAGGGAAAGCCAGTTACTCCAGTGGCAACTG | PCR Products from |
| 77 | GGAACGGCAGGATTATTTTTCCCTTGAGAATG | Cas9-stable HEK cells |
| | ATAATAATTCATTGTTTAGAGGGAGGAAAATC | transfected with dual- |
| | TTCCACAGAACATGGACCTGAAATTCTGACAA | guide RNA pairing, |
| | CCGCTCTTCCTTTTTTCCTCTCTTTCTTTCTCCTT | Up5 + Down5 |
| | AATTTATTTTTTGTTGTACTTATGCATTCATCTC | |
| | TTCAAGATGTTTACTTAGTACCTGATACATTGT | |
| | AGATATTTTCACATTAATGATCCATGTAAATTT | |
| | TATGAGAAACAGTTTTCCTTTACATTCTGCCTT | |
| | CCTCCATTGTTTCTTTCTCTCTCTTAGGAATACT | |
| | TTGTTTATTTTCCTCTCTATTTTTTCCATATTGA | |
| | TTTGAACTATCAATATGACTTTTAATATCCTCT | |
| | AAGCAGAAATGCACGCATGACTTGCACTGGAC | |
| | AAAGCAATATGCCTGGTCACTGCTGAGGTCAA | |
| | CAGGGAGAAAGGACATGGTCTTCCTGCATGAA | |
| | GGACATGAGGA | |
| SEQ ID NO: | AAGAAGAAGAAGAAGAAGAAGAAGAAGAAGA | GAA FGF14 repeat |
| 78 | AGAAGAAGAAGAAGAAGAAGAAGAAGAAGAA | region with human |
| | GAAGAAGAAGAAGAAGAAGAAGAAGAAGAAG | chromosome 13 |
| | AAGAAGAAGAAGAAGAAGAAGAAGAAGAAGA | (chr13: 102,813,925- |
| | AGAAGAAGAAGAAGAAGAAGAAGAAGAA | 102,814,076 (hg19)) |
| SEQ ID NO: | GAAGAAGAAGAAGAAGAAGAAGAA | Example of a |
| 79 | | pathogenic GAA |
| | | FGF14 repeat |
| SEQ ID NO: | AGCATCATTTGGAAGCATAGTGCCAACCTTTTT | Upstream guide 1; |
| 80 | TATTATCTTCTACGCGCACTATATTGGGAACGT | edited sample 199 to |
| | | 264 bp |
| SEQ ID NO: | AGCATCATTTGGAAGCATAGTGCCAACCGCCT | Upstream guide 1; |
| 81 | CCCAGTTAGCTGCAAGTTTCAGAAACTTGTTCT | control sample 198 to |
| | A | 263 bp |
| SEQ ID NO: | CCCCTCATTTGTATGTAGGTTAGTAGCACCCAC | Upstream guide 2; |
| 82 | ATGCCATGCCTAGAGAAATATGGGCTACCGGA | edited sample 146 to |
| | A | 211 bp |
| SEQ ID NO: | CCCCTCATTTGTATGTAGGTTAGTAGCACCGAC | Upstream guide 2; |
| 83 | ACTAAGGGAGAAAATGAATGATGGTTCTAATT | control sample 148 to |
| | C | 213 bp |
| SEQ ID NO: | CTCAATTGCCTTTGCAGCCAGCCAGATCACCTC | Upstream guide 3; |
| 84 | CTGGAAACAGAGGACCCTCAACCCCAACCACC | edited sample 168 to |
| | C | 233 bp |
| SEQ ID NO: | CTCAATTGCCTTTGCAGCCAGCCAGATCACCTC | Upstream guide 3; |
| 85 | CTCGAATACTGGTCCCTGATAACCATCCACCCA | control sample 186 to |
| | | 251 bp |
| SEQ ID NO: | ACATGCCATGCCTTGTGTAATATGGTCTACCGG | Upstream guide 4; |
| 86 | AAAGAGGGGTTTTCCTACTTCTCTTCCATAGAG | edited sample 162 to |
| | | 277 bp |
| SEQ ID NO: | ACATGCCATGCCTTGTGTAATATGGTCTACCGG | Upstream guide 4; |
| 87 | AAGAGGGGTTTTCCTACTTCTCTTCCATAGAGG | control sample 163 to |
| | | 228 bp |
| SEQ ID NO: | CTCAGCAGTGACCAGGCATATTGCTTTGTCCAG | Upstream guide 5; |
| 88 | TGTCAGTTCCATGTTTCAGAAGATATTTGATGA | edited sample 306 to |
| | | 371 bp |
| SEQ ID NO: | CTCAGCAGTGACCAGGCATATTGCTTTGTCCAG | Upstream guide 5; |
| 89 | TGTAATGTCACTTACAGGTAGAAGTTTTCAGAG | control sample 308 to |
| | | 373 bp |
| SEQ ID NO: | GACTTTCTTGTTGAGATGTCTTCGAAAAGCCAC | Downstream guide 1; |
| 90 | ACCTGGAGGTGTGGTTTTCTGAAAGCCCATTTA | edited sample 180 to |
| | | 245 bp |
| SEQ ID NO: | GACTTTCTTGTTGAGATGTCTTCGAAAAGCCAC | Downstream guide 1; |
| 91 | ACTGGAGGTGTGGTTTTCTGAAAGCCCATTTAA | control sample 180 to |
| | | 245 bp |
| SEQ ID NO: | GACAACAGAAGAGTGCACGATGAGCTCTGCCT | Downstream guide 2; |
| 92 | CCGAACTTTCTTGTTGAGATGTCTTCGAAAAGC | edited sample 155 to |
| | C | 220 bp |
| SEQ ID NO: | GACAACAGAAGAGTGCACGATGAGCTCTGCCT | Downstream guide 2; |
| 93 | CCGACTTTCTTGTTGAGATGTCTTCGAAAAGCC | control sample 146 to |
| | A | 211 bp |
| SEQ ID NO: | CGTACCCTTATTTCCTTCCCTTCTTAGAGCCCCC | Downstream guide 3; |
| 94 | CATATTGGGACGTAGGAAGGGGGGAAGGGAG | edited sample 171 to |
| | A | 236 bp |
| SEQ ID NO: | CGTACCCTTATTTCCTTCCCTTCTTAGAGCCCCC | Downstream guide 3; |
| 95 | CATATTGGAGCCTAGGAGAGTGAGATGGAGGA | control sample 72 to |
| | | 137 bp |
| SEQ ID NO: | TTTTTTTTTGCCACCAAAAATAGATGTGGCCAA | Downstream guide 4; |
| 96 | CCGTTTTTCTTCTCCCTTCCCCCCCAACCCCGA | edited sample 112 to |
| | | 177 bp |
| SEQ ID NO: | TTTTTTTTTGCCACCAAAAATAGATGTGGCCAA | Downstream guide 4; |
| 97 | CCGTGAGCATTCAGTCTTCTTCTCCAAGCCCTA | control sample 114 to |
| | | 179 bp |
| SEQ ID NO: | TAATATCCTCTAAGCAGAAATGCACGCATGAC | Downstream guide 5; |
| 98 | TTGGAAGAGTATTTAAAAAGTAGAGGGGGGGT | edited sample 388 to |
| | GG | 453 bp |
| SEQ ID NO: | TAATATCCTCTAAGCAGAAATGCACGCATGAC | Downstream guide 5; |
| 99 | TTGAAGTGGCTTTGAAGACTAACTGAAGTGCT | control sample 387 to |
| | GA | 452 bp |
| SEQ ID NO: | TGTTGGTGGACTAGCACTAGXTGAGCATTCTG | Disease haplotype with |
| 100 | ACAGCTCTTCCCAACTCGG | GAA repeat locus; X = |
| | | any GAA trinucleotide |
| | | repeat |
| SEQ ID NO: | TGTTGGTGGACTAGCACTAGXTGAGCATTCTG | Disease haplotype with |
| 101 | ACAGCCCTTTCCAGCTCGG | GAA repeat locus; X = |
| | | any GAA trinucleotide |
| | | repeat |
| SEQ ID NO: | TGTTGGTGGACTAGCACTAGXTGAGCGTCCGG | Disease haplotype with |
| 102 | GTAGCCCTTTCCAGCTCGG | GAA repeat locus; X = |
| | | any GAA trinucleotide |
| | | repeat |
| SEQ ID NO: | TGTTGGTGGACTAGCACTAGXTGAGCGTCCGG | Disease haplotype with |
| 103 | GTAGCCCTTTCCAACTCGG | GAA repeat locus; X = |
| | | any GAA trinucleotide |
| | | repeat |

TABLES
Table 1. Characteristics of Patients with GAA-FGF14-Related Ataxia

| | French | | German | | Australian | | Indian | | | |
|---|---|---|---|---|---|---|---|---|---|---|
| | Canadian cases | | cases | | cases | | cases | | All cases | |
| | (N = 68)^{a} | | (N = 48)^{b} | | (N = 3) | | (N = 3) | | (N = 122) | |
| Male sex - no. (%) | 32/68 | (47%) | 24/48 | (50%) | 3/3 | (100%) | 3/3 | (100%) | 62/122 | (51%) |
| Inheritance - no. (%) | | | | | | | | | | |
| Sporadic | 10/68 | (15%) | 22/44 | (50%) | 2/3 | (67%) | 2/3 | (67%) | 36/118 | (31%) |
| Familial | 58/68 | (85%) | 22/44 | (50%) | 1/3 | (33%) | 1/3 | (33%) | 82/118 | (69%) |
| Episodic features - no. (%) | 47/67 | (70%) | 6/48 | (12%) | 1/3 | (33%) | 2/3 | (67%) | 56/121 | (46%) |
| Mean age at onset of episodes - ±SD | 54 ± 14 | | 62 ± 7 | | 48 | | 67 | | 55 ± 13 | |
| Mean age at onset of permanent ataxia - ±SD | 59 ± 12 | | 60 ± 11 | | 57 ± 11 | | 62 ± 9 | | 59 ± 11 | |
| Nystagmus - no. (%) | | | | | | | | | | |
| Downbeat nystagmus | 43/65 | (66%) | 5/48 | (10%) | 0/3 | (0%) | 2/3 | (67%) | 50/119 | (42%) |
| Gaze-evoked horizontal nystagmus | 37/65 | (57%) | 25/48 | (52%) | 2/3 | (67%) | 1/3 | (33%) | 65/119 | (55%) |
| Diplopia or visual blurring - no. (%) | 39/66 | (59%) | 16/48 | (33%) | 0/3 | (0%) | 2/3 | (67%) | 57/120 | (48%) |
| Cerebellar dysarthria - no. (%) | 39/68 | (57%) | 21/44 | (48%) | 2/3 | (67%) | 1/3 | (33%) | 63/118 | (53%) |
| Gait ataxia - no. (%) | 65/68 | (96%) | 42/44 | (95%) | 3/3 | (100%) | 3/3 | (100%) | 113/118 | (96%) |
| Appendicular ataxia - no. (%) | 62/68 | (91%) | 27/44 | (61%) | 3/3 | (100%) | 2/3 | (67%) | 94/118 | (80%) |
| Vertigo or dizziness - no. (%) | 21/64 | (33%) | 10/44 | (23%) | 0/3 | (0%) | 2/3 | (67%) | 33/114 | (29%) |
| Postural tremor - no. (%) | 6/60 | (10%) | 11/48 | (23%) | 0/3 | (0%) | 1/3 | (33%) | 18/114 | (16%) |
| Cerebellar atrophy on MRI imaging - no. (%) | 28/44 | (64%) | 34/41 | (83%) | 2/3 | (67%) | 3/3 | (100%) | 67/91 | (74%) |

TABLE 2
Characterization of upstream and downstream gRNAs and primers.

| Upstream gRNAs and Primers | Downstream gRNAs and Primers |
|---|---|
| Upstream Guide #1: | Downstream Guides #1 and #2 (this primer |
| GAAACTTGCAGCTAACTGGG(AGG) | covers both guide regions): |
| SEQ ID NO : 11 | TCTTCGAAAAGCCACACTGG(AGG) |
| 78% specificity, 99% efficiency | SEQ ID NO: 16 |
| Forward Primer: | 76% specificity, 98% efficiency |
| 5′ TGGAAATTTGCTCTGTGGGCT 3′ | CATCTCAACAAGAAAGTCGG(AGG) |
| SEQ ID NO: 21 | SEQ ID NO: 17 |
| Reverse Primer: | 79% specificity, 95% efficiency |
| 5′ CGTTCCCAATATAGTGCGCCT 3′ | Forward Primer: |
| SEQ ID NO: 22 | 5′ AGCATCACTAGAAATTCAGCCAA3′ |
| Position: chr13: 102161798-102162411 | SEQ ID NO: 31 |
| Strand:- | Reverse Primer: |
| Size: 614 bp | 5′ TGGGATAGCAAACACGGCATCA 3′ |
| | SEQ ID NO: 32 |
| | Position: chr13: 102159382-102159777 |
| | Strand:- |
| | Size: 396 bp |
| Upstream Guide #2: | Downstream Guide #3: |
| GTTAGTAGCACCGACACTAA(GGG) | CTCCTAGGCTCCAATATGGG(GGG) |
| SEQ ID NO: 12 | SEQ ID NO: 18 |
| 95% specificity, 86% efficiency | 91% specificity, 78% efficiency |
| Forward Primer: | Forward Primer: |
| 5′ AACCCCTCTTCCGGTAGACC 3′ | 5′ TCTGAGGAAGCGTGGAGGATG 3′ |
| SEQ ID NO: 23 | SEQ ID NO: 33 |
| Reverse Primer: | Reverse Primer: |
| 5′ AGCTGTTTCTCTGGGCCTCTCC 3′ | 5′ AGGTCCATTTACAGCTTCAGTCT 3′ |
| SEQ ID NO: 24 | SEQ ID NO: 34 |
| Position: chr13: 102162735-102163346 | Position: chr13: 102160004-102160248 |
| Strand:- | Strand:- |
| Size: 612 bp | Size: 245 bp |
| Upstream Guide #3: | Downstream Guides #4 and #5 (this primer |
| TCAGGGACCAGTATTCGAGG(AGG) | covers both guide regions): |
| SEQ ID NO: 13 | GAAGACTGAATGCTCACGGT(TGG) |
| 87% specificity, 99% efficiency | SEQ ID NO: 19 |
| Forward Primer: | 91% specificity, 78% efficiency |
| 5′ TCCAGACTTTAAGTGGCCTCCA 3′ | AATGCACGCATGACTTGAAG(TGG) |
| SEQ ID NO: 25 | SEQ ID NO: 20 |
| Reverse Primer: | 78% specificity, 75% efficiency |
| 5′ GGAAGTTTAGAGCAAGTGGAAGG 3′ | Forward Primer: |
| SEQ ID NO: 26 | 5′ ACATTCTGCCTTCCTCCATTGT 3′ |
| Position: chr13: 102163461-102163873 | SEQ ID NO: 35 |
| Strand:- | Reverse Primer: |
| Size: 413 bp | 5′ TCTCACACACCCGGCTCTCTG 3′ |
| | SEQ ID NO: 36 |
| | Position: chr13: 102160458-102161107 |
| | Strand:- |
| | Size: 650 bp |
| Upstream Guide #4: | |
| AATATGGTCTACCGGAAGAG(GGG) | |
| SEQ ID NO: 14 | |
| 92% specificity, 96% efficiency | |
| Forward Primer: | |
| 5′ ACTTCCCCACCCTACACCCT 3′ | |
| SEQ ID NO: 27 | |
| Reverse Primer: | |
| 5′ GCTTCCAAACATAGCCAGACTCT 3′ | |
| SEQ ID NO: 28 | |
| Position: chr13: 102163112-102163711 | |
| Strand:- | |
| Size: 600 bp | |
| Upstream Guide #5: | |
| ACCTGTAAGTGACATTACAC(TGG) | |
| SEQ ID NO: 15 | |
| 82% specificity, 86% efficiency | |
| Forward Primer: | |
| 5′ TGGTCATCAGTAAACACAGTGTC 3′ | |
| SEQ ID NO: 29 | |
| Reverse Primer: | |
| 5′ GGGAAGTGACCAAGGGAGCC 3′ | |
| SEQ ID NO: 30 | |
| Position: chr13: 102163705-102164383 | |
| Strand:- | |
| Size: 679 bp | |

TABLE 3
Sequencing primer targeting within and outside FGF14 gene.

| Primers for sequencing | Primers for sequencing |
|---|---|
| within all of the | outside of the cutting/ |
| cutting/editing regions | editing regions-wildtype |
| F: GGCAAGCTTACACTGACTGA | F: CCAGCACTGTCCAATAGAACTTT |
| SEQ ID NO: 37 | SEQ ID NO: 39 |
| R: ACTGGAGTAACTGTGGCTGT | R: GGTAGTTAATGTCTGTGCTGTCC |
| SEQ ID NO: 38 | SEQ ID NO: 40 |
| chr13: 102161367-102161495 | chr13: 102164666-102164812 |
| 129 bp product | 147 bp product |
| No SNPs | No SNPs |
| 56.6 and 54.8 melting temps | 59.7 and 57.8 melting temps |

TABLE 4
Characteristics of case and control post-mortem cerebellar samples

| | | | | Age at | Post- |
|---|---|---|---|---|---|
| | | | | time of | mortem |
| ID | Control/Case | Genotype | Sex | death | interval |
| SB-C1 | Control | Allele 1: (GAA)₈ | Male | 86 | 7 h 25 |
| | | Allele 2: (GAA)₁₇ | | | |
| SB-C2 | Control | Allele 1: (GAA)₈ | Female | 83 | 7 h 33 |
| | | Allele 2: (GAA)₂₂ | | | |
| SB-C3 | Control | Allele 1: (GAA)₉ | Male | 86 | 7 h 25 |
| | | Allele 2: (GAA)₉(GGA)₁₃(AGA)₇(GAA)₁₃ | | | |
| SB-C4 | Control | Allele 1: (GAA)₉ | Female | 93 | 5 h 30 |
| | | Allele 2: (GAA)₁₀₁ | | | |
| SB-C5 | Control | Allele 1: (GAA)₉ | Female | 83 | 7 h 30 |
| | | Allele 2: (GAA)₃(GAAA)(GAA)₁₀₂ | | | |
| SB-C6 | Control | Allele 1: (GAA)₁₆ | Female | 97 | 7 h 20 |
| | | Allele 2: (GAA)₅(GAG)(GAA)₄₄ | | | |
| SB-C7 | Control | Allele 1: (GAA)₉ | Male | 94 | 15 h 46 |
| | | Allele 2: (GAA)₅₀ | | | |
| SB1 | Case | Allele 1: (GAA)₂₃₃ | Female | 86 | 6 h 25 |
| | | Allele 2: (GAA)₃₀₀ | | | |
| SB2 | Case | Allele 1: (GAA)₉ | Male | 91 | 8 h 15 |
| | | Allele 2: (GAA)₃₅₀ | | | |

Tables 5A and 5B. Experimental conditions used for long-range PCR amplification and RP-PCR

### A. Long-Range PCR

| Reagents | Primers | Cycling conditions |
|---|---|---|
| 1. Phusion Flash High- | F: TGCAAATGAA | 98° C. x 3 min |
| Fidelity PCR Master Mix | GGAAAACTCTT | [98° C. x 10 sec |
| 2X (Thermo-Fisher) | (SEQ ID NO: 41) | 65° C. x 15 sec-Each 2 |
| 2. Primers 1uM | R: CAATGATGAA | cycles decreasing by 1° C. |
| 3. gDNA 40 ng | TTAAGCAGTTCC | 72° C. x 3 min] x 12 |
| | (SEQ ID NO: 42) | cycles |
| | | [98° C. x 10 sec |
| | | 59º C. x 15 sec |
| | | 72° C. x 3 min] x 20 |
| | | cycles |
| | | 72° C. x 5 min |

### B. RP-PCR

| Reagents | Primers | Cycling conditions |
|---|---|---|
| 1. Phusion Flash High- | F: TGCCCACATAGAGCTTAGTCT | 98° C. x 3 min |
| Fidelity PCR Master Mix | (SEQ ID NO: 43) | |
| 2X (Thermo-Fisher) | R: CACGACGTTGTAAAACGAC | [98° C. x 10 sec |
| 2. F primer 1 μM | GAAGAAGAAGAAGAAGAAGAA | 65° C. x 15 sec |
| 3. R primer 0.1 μM | (SEQ ID NO: 44) | 72° C. x 1 min] x |
| 4. M13-FAM 1 μM | M13-FAM: 5′-FAM- | 35 cycles |
| 5. gDNA 40 ng | CACGACGTTGTAAAACGAC | 72° C. x 5 min |
| | (SEQ ID NO: 45) | |

F, forward; gDNA, genomic DNA; PCR, polymerase chain reaction; R, reverse

TABLE 6
Primer sequences used for qPCR experiments

| Gene | Forward primer | Reverse primer | RefSeq | Samples |
|---|---|---|---|---|
| ACTB | ATTGGCAATGAGCGGTT | TGAAGGTAGTTTCGTGGATGC | NM_001101.3 | C, F, |
| | C (SEQ ID NO: 46) | (SEQ ID NO: 53) | | L |
| GAPDH | AGCCACATCGCTCAGAC | GCCCAATACGACCAAATCC | NM_002046.3 | C, |
| | AC (SEQ ID NO: 47) | (SEQ ID NO: 54) | | MIN |
| HPRT1 | TGATAGATCCATTCCTAT | CAAGACATTCTTTCCAGTTAA | NM_000194.2 | C |
| | GACTGTAGA | AGTTG (SEQ ID NO: 55) | | |
| | (SEQ ID NO: 48) | | | |
| YWHAZ | GCAATTACTGAGAGACA | TGGAAGGCCGGTTAATTTT | NM_ | C |
| | ACTTGACA | (SEQ ID NO: 56) | 001135700.1, | |
| | (SEQ ID NO: 49) | | NM_ | |
| | | | 001135701.1, | |
| | | | NM_ | |
| | | | 001135702.1, | |
| | | | NM_ | |
| | | | 003406.3, | |
| | | | NM_ | |
| | | | 145690.2, | |
| | | | NM_ | |
| | | | 001135699.1 | |
| RPL13 | CACCCAGGGAGCTGTTA | CAAGCAAGGTGTCATCGTGT | NM_ | C |
| | CTG (SEQ ID NO: 50) | (SEQ ID NO: 57) | 000977.4, | |
| | | | NM_ | |
| | | | 001243131.1, | |
| | | | NM_ | |
| | | | 033251.2 | |
| UBE2D2 | TTGAATGATCTGGCACG | GCCCCATTATTGTAGCTTGCC | NM_ | C |
| | GGA (SEQ ID NO: 51) | (SEQ ID NO: 58) | 003339.3, | |
| | | | NM_ | |
| | | | 181838.2 | |
| FGF14 | GCAAGCTATGAAAGGGA | GTGCTTTTACTTGGCGTCAC | NM_ | C, F, |
| | ACAG (SEQ ID NO: 52) | (SEQ ID NO: 59) | 175929.3, | L |
| | | | NM_ | |
| | | | 004115.4 | |
| FGF14 | TATTGCAGGCAAGGCTA | GTTTTCACTCCCTGGATGGCA | NM_ | MN |
| | CTACTTG (SEQ ID NO: 60) | AC (SEQ ID NO: 63) | 175929.3, | |
| | | | NM_ | |
| | | | 004115.4 | |
| FGF14 | CATATGCTGCAGTGTCTT | GTAGTAGCCTTGCCTGCAATA | NM_ | C, F, |
| | TGTG (SEQ ID NO: 61) | (SEQ ID NO: 64) | 175929.3 | MN, L |
| FGF14 | GCAACCTGGTGGATATC | ACCTGGTCACTATACCCTTGA | NM_ | C, F, |
| | TTCTC (SEQ ID NO: 62) | (SEQ ID NO: 65) | 004115.4 | MN, L |

C, post-mortem cerebellar tissue; F, fibroblasts; MN, iPSC-derived motor neurons; L, lymphoblasts

TABLE 7
Segregating repeat expansions identified by
ExpansionHunter Denovo in six French-
Canadian LOCA cases-Screening for
repeat expansions in PCR-free whole-
genome sequencing by ExpansionHunter
Denovo in six French-Canadian LOCA
cases compared to 1000 Genome Project
control cohort.

| | | | | Reason |
|---|---|---|---|---|
| Genomic region (Hg19) | Motif | Location | Gene | filtered out |
| chr13: 102813925-102814074 | AAG | Intronic | FGF14 | N/A |
| chr1: 142535433-142539733 | ACTCC | Intergenic | EMBP1; | Intergenic |
| | | | RP11- | |
| | | | 782C8.1 | |
| chr20: 62122604-62124183 | ATCC | Intronic | EEF1A2 | Mean IRR in |
| | | | | cases is 4.4 |
| chr1: 112929991-112931170 | AAGG | Intergenic | snoU13; | Intergenic |
| | | | CTTNBP2NL | |
| chr16: 57762434-57764056 | ACCC | Intronic | DRC7 | Mean IRR in |
| | | | | cases is 2.8 |
| chr1: 8404492-8405558 | ATCC | Intergenic | SLC45A1; | Intergenic |
| | | | RERE | |
| chr12: 24459840-24461128 | AAGG | Intronic | SOX5 | Mean IRR in |
| | | | | cases is 3.4 |
| chr2: 206247390-206248233 | AGAT | Intronic | PARD3B | Mean IRR in |
| | | | | cases is 2.6 |

IRR = in-repeat reads

1. A gene editing system comprising:
a first guide RNA (gRNA) or a first nucleic acid sequence encoding the first gRNA;
a second gRNA or a second nucleic acid sequence encoding the second gRNA; and
an endonuclease or a third nucleic acid sequence encoding the endonuclease;
wherein the first and second gRNAs target the endonuclease to a first and a second genomic loci in a nucleus of a cell that together flank a GAA repeat in intron 1 of a fibroblast growth factor 14 (FGF14) gene.

2. The gene editing system of claim 1, wherein the gene editing system is a CRISPR gene editing system comprising a Cas9 endonuclease or a Cpf1 endonuclease.

3. (canceled)

4. The gene editing system of claim 1, wherein the first gRNA comprises SEQ ID NO: 1, SEQ ID NO: 2, SEQ ID NO: 3, SEQ ID NO: 4, or SEQ ID NO: 5.

5. The gene editing system of claim 1, wherein the second gRNA comprises SEQ ID NO: 6, SEQ ID NO: 7, SEQ ID NO: 8, SEQ ID NO: 9, or SEQ ID NO: 10.

6. The gene editing system of claim 1, wherein the first and second genomic loci are upstream (5′) of the GAA repeat and downstream (3′) of the GAA repeat.

7. The gene editing system of claim 1, wherein the gene editing system removes at least 100 GAA repeats.

8. (canceled)

9. (canceled)

10. A method of gene editing of a fibroblast growth factor 14 (FGF14) gene in a cell or a subject, the method comprising:
targeting a first guide RNA (gRNA) and an endonuclease to a first genomic loci of the FGF14 gene,
targeting a second gRNA and the endonuclease to a second genomic loci of the FGF14 gene, and
removing a GAA repeat in intron 1 of the FGF14 gene.

11. The method of claim 10, wherein the method comprises a CRISPR gene editing system comprising a Cas9 endonuclease or a Cpf1 endonuclease.

12. (canceled)

13. The method of claim 10, wherein the first gRNA comprises SEQ ID NO: 1, SEQ ID NO: 2, SEQ ID NO: 3, SEQ ID NO: 4, or SEQ ID NO: 5.

14. The method of claim 10, wherein the second gRNA comprises SEQ ID NO: 6, SEQ ID NO: 7, SEQ ID NO: 8, SEQ ID NO: 9, or SEQ ID NO: 10.

15. The method of claim 10, wherein the method removes at least 100 GAA repeats.

16. (canceled)

17. The method of claim 10, wherein the first and second genomic loci are upstream (5′) of the GAA repeat and downstream (3′) of the GAA repeat.

18. (canceled)

19. (canceled)

20. A method of treating or preventing a late-onset cerebellar ataxia (LOCA), the method comprising:
administering to a subject in need thereof a gene editing system to at least one cell in the subject, wherein the gene editing system comprises:
a first guide RNA (gRNA) or a first nucleic acid sequence encoding the first gRNA;
a second gRNA or a second nucleic acid sequence encoding the second gRNA; and
an endonuclease or a third nucleic acid sequence encoding the endonuclease;
wherein the first and second gRNAs target the endonuclease to a first and a second genomic loci in a nucleus of the cell that together flank a GAA repeat in intron 1 of a fibroblast growth factor 14 (FGF14) gene.

21. The method of claim 20, wherein the method comprises a CRISPR gene editing system comprising a Cas9 endonuclease or a Cpf1 endonuclease.

22. (canceled)

23. The method of claim 20, wherein the first gRNA comprises SEQ ID NO: 1, SEQ ID NO: 2, SEQ ID NO: 3, SEQ ID NO: 4, or SEQ ID NO: 5.

24. The method of claim 20, wherein the second gRNA comprises SEQ ID NO: 6, SEQ ID NO: 7, SEQ ID NO: 8, SEQ ID NO: 9, or SEQ ID NO: 10.

25. The method of claim 20, wherein the endonuclease partially or completely removes the GAA repeat at intron 1 of the FGF14 gene.

26. The method of claim 20, wherein the FGF14 gene is reassembled by a recombination event.

27. The method of claim 20, wherein the gene editing system removes at least 100 GAA repeats.

28. (canceled)

29. The method of claim 20, wherein the first and second genomic loci are upstream (5′) of the GAA repeat and downstream (3′) of the GAA repeat.

30. (canceled)

31. (canceled)
